SEQUENCE LISTING (53 sequences)

SEQ ID NO: 1 - protein, 63 aa, synthetic construct
  VARIANT 8: note = X is D or S
  VARIANT 16: note = X is R or K
  VARIANT 44: note = X is E, Q, or M
  VARIANT 46: note = X is D, N, Q, or K
  VARIANT 47: note = X is E, N, Q, D, A, or T
  VARIANT 50: note = X is E, A, or Q
  VARIANT 53: note = X is D or E
  VARIANT 62: note = X is Y, W, or absent
  VARIANT 63: note = X is G or absent
DDKAACAXGIAAVKAXVEKLAPEAVPQKLKRALKIAEREQGEGXFXXCLXALXDAKRALPKXX

SEQ ID NO: 2 - protein, 94 aa, synthetic construct
MMRTRTSLAVPRGFRGSALLALVVLATPALADDKAACADGIAAVKARVEKLAPEAVPQKLKRALKIAEREQGEGEFDECLEALDDAKRALPKYG

SEQ ID NO: 3 - protein, 94 aa, synthetic construct
MMRTRTSLAVPRGFRGSALLALVVLATPALADDKAACASGIAAVKARVEKLAPEAVPQKLKRALKIAEREQGEGEFDECLEALDDAKRALPKYG

SEQ ID NO: 4 - protein, 94 aa, synthetic construct
MMRTRTSLAVPRGFRGSALLALVVLATPALADDKAACADGIAAVKAKVEKLAPEAVPQKLKRALKIAEREQGEGEFDECLEALDDAKRALPKYG

SEQ ID NO: 5 - protein, 94 aa, synthetic construct
MMRTRTSLAVPRGFRGSALLALVVLATPALADDKAACADGIAAVKARVEKLAPEAVPQKLKRALKIAEREQGEGQFDECLEALDDAKRALPKYG

SEQ ID NO: 6 - protein, 94 aa, synthetic construct
MMRTRTSLAVPRGFRGSALLALVVLATPALADDKAACADGIAAVKARVEKLAPEAVPQKLKRALKIAEREQGEGEFDACLEALDDAKRALPKYG

SEQ ID NO: 7 - protein, 94 aa, synthetic construct
MMRTRTSLAVPRGFRGSALLALVVLATPALADDKAACADGIAAVKARVEKLAPEAVPQKLKRALKIAEREQGEGEFDNCLEALDDAKRALPKYG

SEQ ID NO: 8 - protein, 94 aa, synthetic construct
MMRTRTSLAVPRGFRGSALLALVVLATPALADDKAACADGIAAVKARVEKLAPEAVPQKLKRALKIAEREQGEGEFDQCLEALDDAKRALPKYG

SEQ ID NO: 9 - protein, 94 aa, synthetic construct
MMRTRTSLAVPRGFRGSALLALVVLATPALADDKAACADGIAAVKARVEKLAPEAVPQKLKRALKIAEREQGEGEFDDCLEALDDAKRALPKYG

SEQ ID NO: 10 - protein, 94 aa, synthetic construct
MMRTRTSLAVPRGFRGSALLALVVLATPALADDKAACADGIAAVKARVEKLAPEAVPQKLKRALKIAEREQGEGQFDACLEALDDAKRALPKYG

SEQ ID NO: 11 - protein, 94 aa, synthetic construct
MMRTRTSLAVPRGFRGSALLALVVLATPALADDKAACADGIAAVKARVEKLAPEAVPQKLKRALKIAEREQGEGQFDNCLEALDDAKRALPKYG

SEQ ID NO: 12 - protein, 94 aa, synthetic construct
MMRTRTSLAVPRGFRGSALLALVVLATPALADDKAACADGIAAVKARVEKLAPEAVPQKLKRALKIAEREQGEGEFNECLAALDDAKRALPKYG

SEQ ID NO: 13 - protein, 94 aa, synthetic construct
MMRTRTSLAVPRGFRGSALLALVVLATPALADDKAACADGIAAVKARVEKLAPEAVPQKLKRALKIAEREQGEGEFQECLQALDDAKRALPKYG

SEQ ID NO: 14 - protein, 94 aa, synthetic construct
MMRTRTSLAVPRGFRGSALLALVVLATPALADDKAACADGIAAVKARVEKLAPEAVPQKLKRALKIAEREQGEGEFKECLQALDDAKRALPKYG

SEQ ID NO: 15 - protein, 94 aa, synthetic construct
MMRTRTSLAVPRGFRGSALLALVVLATPALADDKAACADGIAAVKARVEKLAPEAVPQKLKRALKIAEREQGEGQFDTCLEALDDAKRALPKYG

SEQ ID NO: 16 - protein, 94 aa, synthetic construct
MMRTRTSLAVPRGFRGSALLALVVLATPALADDKAACADGIAAVKARVEKLAPEAVPQKLKRALKIAEREQGEGQFDACLEALEDAKRALPKYG

SEQ ID NO: 17 - protein, 94 aa, synthetic construct
MMRTRTSLAVPRGFRGSALLALVVLATPALADDKAACADGIAAVKARVEKLAPEAVPQKLKRALKIAEREQGEGMFDACLEALDDAKRALPKYG

SEQ ID NO: 18 - protein, 92 aa, Methylorubrum extorquens
MMRTRTSLAVPRGFRGSALLALVVLATPALADDKAACADGIAAVKARVEKLAPEAVPQKLKRALKIAEREQGEGEFDECLEALDDAKRALPK

SEQ ID NO: 19 - protein, 81 aa, Hansschlegelia quercus
MRIYLLASLALLATLPAARADDAADCDAGIAMIRMEQAKEHGRATTESLKTALRVAEREKGEKEYDECLDAVADARKALKK

SEQ ID NO: 20 - protein, 83 aa, Xanthobacteraceae bacterium
MNQLNAALLAATLIGVSASAVRADDAKVCTDGIAMIKAEIAKTPPKATLDKLNKALKGAEREYGEKEFDECVDFVNDAKKAKG

SEQ ID NO: 21 - protein, 83 aa, Hansschlegelia beijingensis
MMKVFLLAAAGLAFAGATAQADEASDCDAGIAMIRAEAAGSHPPAVADSLKTALRVAEREQGEKEYDECLDAIDDAKKALNKK

SEQ ID NO: 22 - protein, 84 aa, Microvirga zambiensis
MRPLLMSCLVLTTFASSVMPAVADDQADCTAGIAMIKAELDKKPPQTTLTALQRALRSAERELKEAEFDECVDAVDDAKKALGR

SEQ ID NO: 23 - protein, 90 aa, Ancylobacter mangrovi
MRERTLRILLSAALLAGAATFATLGSALADDAKDCSDGVAMIKAEIAKGPPKATLDKLNKALRGAQREMGEGEYDECLDFVGDAKKAIKG

SEQ ID NO: 24 - protein, 84 aa, Prosthecodimorpha staleyi
MLRPALVVAALLIASGPALAADDLASCTKGIAFIKAEIAKNPPAPVLTRLKKALKDANRELGEGEFDECMDAVRDAEKTTGRKS

SEQ ID NO: 25 - protein, 85 aa, Ancylobacter crimeensis
MRYTGLKLALGLAAGLAFGGVALADDAKTCNDGIAMIKAEIAKKPPKATLDKLTKALKGAEREHGEKEYDECVDYIKDAQKAVGG

SEQ ID NO: 26 - protein, 84 aa, Salinarimonas soli
MRRTVVPMLGLAAALAAPVPALADDQADCVAGIAMIRAEIAKNPPQATLTALQRALRSAERELKEAEFDECVDAVNDARKALRR

SEQ ID NO: 27 - protein, 83 aa, Ancylobacter lacus
MTRTLATAAAVLVLGLGTAAFADDAKQCSDGITFLKAEAAKNPPKATLDKIKKALKGAERELGEKEYDECIDYVNDGKKAVSG

SEQ ID NO: 28 - protein, 84 aa, Enterovirga sp.
MGRILLSVSLVIAAGMIGGRPASADDRSECAAGIAMLEAELSKATGAVRTKVERELRVARREQAEGEFDECMDAIRAARPALRQ

SEQ ID NO: 29 - protein, 82 aa, Azospirillum sp.
MRTPTFAVLTATLLATPALADDQSECVAGIGFIKAEIAKAPPQPILDALKKQLRNAEREQREKEYDECLDAVTAARKAVAAK

SEQ ID NO: 30 - protein, 81 aa, Hansschlegelia zhihuaiae
MLALGLVAAAALACPEARADAAADCDSGIAMITAESAKEQPGPVADALKTALRVAKREKGEQEYDECLDAVEDARKALKKK

SEQ ID NO: 31 - protein, 87 aa, Ancylobacter novellus
MTGLRTFAASLAFLSACVVAAGSARADDAADCDAGIEMITAEIAGEHPKATADALRTALRVAKREKGEKEYDECLDAVADAKKALRK

SEQ ID NO: 32 - protein, 87 aa, Chenggangzhangella methanolivorans
MSARTHSALTGLALLAALALPAPAFADDAADCDAGIAMISSEVAKEHPKATADALKTALRVAKREKGEKEYDECLDAVADAKKALGR

SEQ ID NO: 33 - protein, 86 aa, Chelatococcus sambhunathii
MSALQHSFRALTIAAALSVSAPAFADAAADCDAGIEMISAEVAKEHPKAAAEALKKALKVAKREKGEKEYDECLDAVADAKKALGR

SEQ ID NO: 34 - protein, 63 aa, synthetic construct
DDKAACADGIAAVKARVEKLAPEAVPQKLKRALKIAEREQGEGEFDECLEALDDAKRALPKYG

SEQ ID NO: 35 - protein, 63 aa, synthetic construct
DDKAACASGIAAVKARVEKLAPEAVPQKLKRALKIAEREQGEGEFDECLEALDDAKRALPKYG

SEQ ID NO: 36 - protein, 63 aa, synthetic construct
DDKAACADGIAAVKAKVEKLAPEAVPQKLKRALKIAEREQGEGEFDECLEALDDAKRALPKYG

SEQ ID NO: 37 - protein, 63 aa, synthetic construct
DDKAACADGIAAVKARVEKLAPEAVPQKLKRALKIAEREQGEGQFDECLEALDDAKRALPKYG

SEQ ID NO: 38 - protein, 63 aa, synthetic construct
DDKAACADGIAAVKARVEKLAPEAVPQKLKRALKIAEREQGEGEFDACLEALDDAKRALPKYG

SEQ ID NO: 39 - protein, 63 aa, synthetic construct
DDKAACADGIAAVKARVEKLAPEAVPQKLKRALKIAEREQGEGEFDNCLEALDDAKRALPKYG

SEQ ID NO: 40 - protein, 63 aa, synthetic construct
DDKAACADGIAAVKARVEKLAPEAVPQKLKRALKIAEREQGEGEFDQCLEALDDAKRALPKYG

SEQ ID NO: 41 - protein, 63 aa, synthetic construct
DDKAACADGIAAVKARVEKLAPEAVPQKLKRALKIAEREQGEGEFDDCLEALDDAKRALPKYG

SEQ ID NO: 42 - protein, 63 aa, synthetic construct
DDKAACADGIAAVKARVEKLAPEAVPQKLKRALKIAEREQGEGQFDACLEALDDAKRALPKYG

SEQ ID NO: 43 - protein, 63 aa, synthetic construct
DDKAACADGIAAVKARVEKLAPEAVPQKLKRALKIAEREQGEGQFDNCLEALDDAKRALPKYG

SEQ ID NO: 44 - protein, 63 aa, synthetic construct
DDKAACADGIAAVKARVEKLAPEAVPQKLKRALKIAEREQGEGEFNECLAALDDAKRALPKYG

SEQ ID NO: 45 - protein, 63 aa, synthetic construct
DDKAACADGIAAVKARVEKLAPEAVPQKLKRALKIAEREQGEGEFQECLQALDDAKRALPKYG

SEQ ID NO: 46 - protein, 63 aa, synthetic construct
DDKAACADGIAAVKARVEKLAPEAVPQKLKRALKIAEREQGEGEFKECLQALDDAKRALPKYG

SEQ ID NO: 47 - protein, 63 aa, synthetic construct
DDKAACADGIAAVKARVEKLAPEAVPQKLKRALKIAEREQGEGQFDTCLEALDDAKRALPKYG

SEQ ID NO: 48 - protein, 63 aa, synthetic construct
DDKAACADGIAAVKARVEKLAPEAVPQKLKRALKIAEREQGEGQFDACLEALEDAKRALPKYG

SEQ ID NO: 49 - protein, 63 aa, synthetic construct
DDKAACADGIAAVKARVEKLAPEAVPQKLKRALKIAEREQGEGMFDACLEALDDAKRALPKYG

SEQ ID NO: 50 - protein, 61 aa, Methylorubrum extorquens
DDKAACADGIAAVKARVEKLAPEAVPQKLKRALKIAEREQGEGEFDECLEALDDAKRALPK

SEQ ID NO: 51 - protein, 31 aa, Methylorubrum extorquens
MMRTRTSLAVPRGFRGSALLALVVLATPALA

SEQ ID NO: 52 - protein, 94 aa, synthetic construct
  VARIANT 39: note = X is D or S
  VARIANT 47: note = X is R or K
  VARIANT 65: note = X is E, Q, or M
  VARIANT 67: note = X is D, N, Q, or K
  VARIANT 68: note = X is E, N, Q, D, A, or T
  VARIANT 71: note = X is E, A, or Q
  VARIANT 74: note = X is D or E
  VARIANT 93: note = X is Y, W, or absent
  VARIANT 94: note = X is G or absent
MMRTRTSLAVPRGFRGSALLALVVLATPALADDKAACAXGIAAVKAXVEKLAPEAVPQKLKRALKIAEREQGEGXFXXCLXALXDAKRALPKXX

SEQ ID NO: 53 - protein, 11 aa, synthetic construct
  VARIANT 3: note = X is any amino acid
  VARIANT 4: note = X is any amino acid
  VARIANT 6: note = X is any amino acid
  VARIANT 8: note = X is F or Y
REXXEXEXDEC

(19) United States
(12) Patent Application Publication
(10) Pub. No.: US 2026/0118347 A1
(43) Pub. Date: Apr. 30, 2026

(54) LANTHANIDE-TEMPLATED PROTEIN DIMERIZATION AND FINER RARE EARTH SEPARATION

(71) Applicant: The Penn State Research Foundation, University Park, PA (US)

(72) Inventors: Wyatt Bolt Larrinaga, University Park, PA (US); Jonathan Jung, University Park, PA (US); Amie Kathleen Boal, University Park, PA (US); Joseph Alfred Cotruvo, JR., University Park, PA (US)

(21) Appl. No.: 19/145,047

(22) PCT Filed: Sep. 19, 2024
(86) PCT No.: PCT/US2024/047567

§ 371 Date: Jul. 1, 2025

Related U.S. Application Data
(60) Provisional application No. 63/583,690, filed on Sep. 19, 2023.
(60) Provisional application No. 63/644,423, filed on May 8, 2024.

(51) Int. Cl. G01N 33/53; C07K 14/195; G01N 33/18; G01N 33/543

(52) U.S. Cl. CPC G01N 33/5308; C07K 14/195; G01N 33/1813; G01N 33/543; C07K 2319/02; G01N 2333/195

(57) ABSTRACT

Provided are proteins and peptides suitable for binding lanthanide ions and/or actinide ions. Proteins of the present disclosure may contain or be one of the following sequences: DDKAACAX¹GIAAVKAX²VEKLAPEAVPQKLKRALKIAEREQGEGX ³FX⁴X⁵CLX⁶ALX⁷DAKRALPKX⁸X⁹ (SEQ ID NO:1) or MMRTRTSLAVPRGFRGSALLADDKAACAX¹GIAAVKAX²VEKLA-PEAVPQKLKRALKIAEREQGEGX³FX⁴X⁵CLX⁶ALX⁷DAKRALPKX⁸X⁹ (SEQ ID NO:52), where X¹ is D or S; X² is R or K; X³ is E, Q, or M; X⁴ is D, N, Q, or K; X⁵ E, N, Q, D, A, or T; X⁶ is E, A, or Q; X⁷ is D or E; X⁸ is Y, W, or absent; and X⁹ is G or absent, or a protein having at least 70% identity to SEQ ID NO:1 or 52. Proteins or peptides of the present disclosure may contain a metal-binding motif or be REX¹X²EX³EX⁴DEC (SEQ ID NO:53), where X¹ is any amino acid; X² is any amino acid, (e.g., G, A, K, R); X³ is any amino acid, (e.g., G, A, or K); and X⁴ is F or Y; and the C forms a disulfide bond with another cysteine residue elsewhere in the peptide. Also provided are methods of using any one of the proteins or peptides, methods of making any one of the proteins or peptides, and kits.

### CROSS REFERENCE TO RELATED APPLICATIONS

This application claims the benefit of priority to U.S. Provisional Application No. 63/583,690, filed on Sep. 19, 2023, and to U.S. Provisional Application No. 63/644,423, filed on May 8, 2024, the disclosure of which is hereby incorporated by reference.

### STATEMENT REGARDING FEDERALLY SPONSORED RESEARCH

This invention was made with government support under Grant No. CHE1945015 awarded by the National Science Foundation, under Grant No. GM119707 awarded by the National Institutes of Health and under Grant No. DE-AC52-07NA27344 awarded by the Department of Energy. The Government has certain rights in the invention.

### SEQUENCE LISTING

The instant application contains a Sequence Listing, which has been submitted in xml format and is hereby incorporated by reference in its entirety. Said .xml copy was created on Sep. 19, 2024, is named “074339_00276_ST26.xml”, and is 58.845 bytes in size.

### BACKGROUND OF THE DISCLOSURE

Owing to the, on average, ˜0.01 Å difference in ionic radius between adjacent lanthanides, rare earth (RE) separations are challenging but critically important for the clean-energy economy. Standard industrial ligands have separation factors (SFs) for adjacent lanthanide (Ln^{III}) ions as low as 1.1. It is also important that ligands disfavor binding of the largest REs, La^{III} and Ce^{III}, as these elements can comprise >70% of many feedstocks but have little value, whereas slightly smaller Pr^{III} and Nd^{III} are substantially more desirable. Much recent work has been devoted to creative approaches to improve RE separations. Synthetic molecular approaches to amplify SFs include using rigid, pre-organized ligands to impart higher selectivity over part of the series: tug-of-war involving ligands with opposite selectivity trends; ligands with unusual selectivity trends; and reactivity-based separations. Several of these ligands have promising SFs but may bind very tightly or exhibit slow equilibration kinetics, both of which may be sub-optimal given the need for multiple adsorption/desorption stages.

Another approach uses dimerizing synthetic ligand: RE complexes. Biology has also landed on a similar concept. Although the archetypal highly selective lanthanide-binding protein, lanmodulin (LanM), from Methylobacterium (Methylorubrum) extorquens (Mex-LanM) is purely monomeric, a LanM from another organism, Hans-LanM, dimerizes in a manner sensitive to the ionic radius of the RE. This sensitivity likely results from a carboxylate shift that affects coordination number at a metal-binding site in one monomer that connects to the other monomer via a hydrogen-bonding network across an extensive dimer interface. However, the SFs of dimerizing small-molecule and natural and engineered protein-based systems, as well as in monomeric LanMs due to their multiple metal sites, are dampened by formation of mixed-metal complexes. Therefore, greater radius sensitivity, and thus higher SFs, might be better achieved by a single, interfacial metal site.

Shortly after reporting LanM, our group identified a 6.8-kDa periplasmic protein of unknown function in M. extorquens, META1p1781 (LanD, which we now name “landiscernin,” for lanthanide-discerning protein), as part of the lanM gene cluster that included machinery for lanthanide uptake (FIG. 9) (Mattocks et al. JACS 2019, 141, 2857). The lanD gene partially overlaps with the gene encoding the cytosolic component of the ATP-binding cassette (ABC) transporter for import of lanthanides to the cytosol, suggesting a potential role for LanD as a chaperone or accessory protein. Supporting this hypothesis, preliminary studies showed that LanD shares LanM's preference for binding of larger REs. Competition assays with xylenol orange indicated the protein can outcompete the dye for the lighter lanthanides (La—Gd, not Ho and heavier; Tb and Dy were not tested), suggesting a tighter than ˜10 μM affinity at pH 6. Although the stoichiometry was not fully clear (between 0.5 and 1 equivalent binding), the data were interpreted as 1 equivalent under the XO assay conditions (˜10 μM protein). Unlike LanM, LanD lacks EF-hand sequence motifs, indicating a heretofore uncharacterized Ln^{III}-binding site.

The logic of periplasmic trafficking of lanthanide ions in lanthanide-utilizing bacteria is similarly uncharacterized. A Ln^{III}-metallophore complex has been inferred to be involved in uptake and the likely solute-binding protein (META1p1778) for that complex has been isolated. LanM's preferential recognition of Nd^{III} and Sm^{III} has been studied extensively but its biological function is less well understood. LanD and another recently discovered protein, LanP, bind lanthanides but their functions are not established. How these players fit together is also unknown. Importantly, only La^{III}, Ce^{III}, Pr^{III}, and Nd^{III} (called “light lanthanides” herein) are imported efficiently into the cytosol in M. extorquens to support lanthanide-dependent growth. In principle, this result could be explained by a metallophore or outer-membrane transporter specific for these particular REs, but recent work implies such systems cannot alone account for the specificity of cytosolic lanthanide import.

### SUMMARY OF THE DISCLOSURE

The present disclosure provides proteins that bind rare earth metal ions. Also provided are devices and kits comprising a protein of the present disclosure. Also provided are methods of using the proteins and devices.

In an aspect, the present disclosure provides proteins that bind metal ions (e.g., lanthanide ions and/or actinide ions).

A metal-binding protein of the present disclosure may comprise signal sequence:

(MMRTRTSLAVPRGFRGSALLALVVLATPALADDKAACADGIAAV
KARVEKLAPEAVPQKLKRALKIAEREQGEGEFDECLEALDDAKRA
LPKYG (SEQ ID NO: 2))

or exclude a signal sequence:

(SEQ ID NO: 34)
DDKAACADGIAAVKARVEKLAPEAVPQKLKRALKIAEREQGEGEFDE
CLEALDDAKRALPKYG,

where the signal sequence or signal peptide has the following sequence:

(SEQ ID NO: 51)
MMRTRTSLAVPRGFRGSALLALVVLATPALA

In various examples, a metal-binding protein of the present disclosure has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99%) identity to SEQ ID NO:2 or SEQ ID NO:34. In various examples, a protein of the present disclosure may be affixed or disposed on a substrate. The Signal Peptide may be removed prior to using the protein.

In various examples, a metal-binding protein of the present disclosure has the following sequence or comprises the following sequence:

```
  (SEQ ID NO: 1)
DDKAACAX¹GIAAVKAX²VEKLAPEAVPQKLKRALKIAEREQGEG
X³FX⁴X⁵CLX⁶ALX⁷DAKRALPKX⁸X⁹
or
  (SEQ ID NO: 52)
MMRTRTSLAVPRGFRGSALLADDKAACAX¹GIAAVKAX²VEKLAP
EAVPQKLKRALKIAEREQGEGX³FX⁴X⁵CLX⁶ALX⁷DAKRALPKX⁸X⁹,
```

where X¹ is D or S: X² is R or K: X³ is E, Q, or M: X⁴ is D, N, Q, or K: X⁵ E, N, Q, D, A, or T: X⁶ is E, A, or Q: X⁷ is D or E; X⁸ is Y, W, or absent; and X⁹ is G or absent. In various examples, a metal-binding protein may have at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99%) identity to SEQ ID NO: 1 or SEQ ID NO:52. In various examples, the signal sequence may be removed prior to using the protein.

In various examples, a metal-binding protein comprises one or more metal-binding or metal coordination motifs. The one or more metal-binding or metal coordination motifs may have the following sequence: REX¹X²EX³EX⁴DEC (SEQ ID NO:53), where X¹ is any amino acid: X² is any amino acid, (e.g., G, A, K, R): X³ is any amino acid, (e.g., G, A, or K); and X⁴ is F or Y; and the C forms a disulfide bond with another cysteine residue elsewhere in the peptide. In various examples, any one or more of the non-X residues may be substituted with an amino acid that is isoelectronic, isostructural, or be replaced with alanine. For example, any D can be replaced with N, E, or A; any E can be replaced with Q, D, or A; or R can be replaced with K. In various examples, any one or more of the glutamic acid residues may be replaced with a softer Lewis base amino acid residue (e.g., C, M, Q, or H) (e.g., RX⁵X¹X²X⁶X³X¹X⁴DX⁸C, where X⁵, X⁶, X⁷, and X⁸ are independently chosen from E, C, M, Q, or H.

In an aspect, the present disclosure provides various methods of using the proteins and/or devices of the present disclosure. A method of the present disclosure may be for binding one or more lanthanides and/or actinides or for detecting and/or quantifying the amount of one or more lanthanides and/or actinides.

In an aspect, the present disclosure provides kits. The kits may comprise a protein of the present disclosure. The kit may further comprise instructions for use. Additionally, a kit may comprise a substate, instructions and materials to conjugate or otherwise attach the protein to the substrate.

In an aspect, the present disclosure provides methods of making a protein of the present disclosure.

### BRIEF DESCRIPTION OF THE FIGURES

For a fuller understanding of the nature and objects of the disclosure, reference should be made to the following detailed description taken in conjunction with the accompanying figures.

FIG. 1. X-ray crystal structures of LanD in the apo state and the La(III)- or Eu(III)-bound state. Panel A shows X-ray crystal structure of LanD apoprotein depicting the dimer interface, with hydrogen bonds formed between D39 and R47 residues between monomer units. Panel B shows X-ray crystal structure of LanD with La(III) bound depicting the holo-dimer interface. A coordinated water molecule (partially occupied) is shown as a red sphere. Panel C shows detail of the La(III) coordination site from B, showing 9-coordination of the La(III) ion. Panel D shows X-ray crystal structure of LanD with Eu(III) bound, depicting a similar holo-dimer interface as in B. Panel E shows detail of the Eu(III) coordination site from D, showing 8-coordination of the Eu(III) ion and an absence of coordinated solvent. See FIG. 4 for more details.

FIG. 2. Dimer dissociation of apo-(Panel A) and Nd-bound (Panel B) LanD. Panel A shows Top: Representative ITC trace for titration of 800 μM apoprotein into Tris buffer. Bottom: Thermogram derived from the data above fitted to the dimer dissociation model using the Nano Analyze software. Panel B shows Top: Representative ITC trace for titration of 300 μM Nd(III)-LanD₂ into Tris buffer. Bottom: Thermogram derived from the data above fitted to the dimer dissociation model using the NanoAnalyze software.

FIG. 3. Ln(III) binding to LanD studied by isothermal titration calorimetry (ITC). (Top) A representative ITC curve is shown for a titration of 400 μM CeCl₃ into 60 μM LanD (no citrate) in Tris buffer, with integrated heats for each titration. The main response is at 0.5 equiv, but cannot be fitted easily because of the contribution from a background endothermic event. (Bottom) Data from titrations buffered with 500 μM citrate. Integrated heats for citrate-buffered ITC titrations versus calculated free Ln concentrations (excluding contributions of protein binding, see comments in Example 1 for interpretation).

FIG. 4. X-ray crystal structures of apo- and La^{III}-bound LanD reveal a metal-centered dimer. Panel A shows X-ray crystal structure of the apo-LanD dimer showing the disulfide-stabilized three-helix bundle fold and the apo dimer interface, stabilized by hydrogen bonds between Asp39 in one monomer and Arg47 in the other. The apo dimer interface is located on the first helix, away from the Ln^{III} binding site. “N” and “C” denote the N- and C-termini, respectively. Panel B shows overall view of the La^{III}-LanD structure showing the lanthanide-binding site near the end of helix 2. Panel C shows enlarged depiction of the La^{III} site in LanD. Dashed lines show coordination bonds. Panel D shows view of key second-sphere amino acids near the lanthanide-binding site in La^{III}-LanD. The metal binding site is surrounded by additional polar side chains, including the strictly conserved residues. Arg69 and Glu78.

FIG. 5. Biochemical and structural analysis of lanthanide-sensitive dimerization. Panel A shows Dimer dissociation constants (K_{dimer} values, black squares) for La^{III}-, Ce^{III}-, Nd^{III}-, Eu^{III}-, and Ho^{III}-LanD, determined by ITC. The K_{dimer} for metal-dependent dimerization of Eu^{III}-LanD as determined by luminescence lifetime analysis is shown as a red circle. (Panels B, C, and D show comparison of the metal-binding sites across a series of lanthanides bound to LanD. Contraction of the ionic radius across the lanthanide series results in loss of coordinated solvent and shorter metal-ligand bonds.

FIG. 6. LanD monomer preferentially binds Nd^{III}-Eu^{III}. Panel A shows determination of q values for 20 and 350 μM Eu^{III}-LanD, supporting coordination with ˜4 solvent molecules in the monomeric state. Panel B shows a plot of K_{d1} values for Ln^{III}-LanD.

FIG. 7. Interfacial mutations enable light lanthanide separations. Panel A K_{d1} for each Ln^{III} ion relative to Eu^{III} for LanD-E75Q, from competitive luminescence titrations (black squares). K_{dimer} values for LanD-E75Q/E78A, measured by ITC (red circles). Panel B shows luminescence competition titration with 10 μM LanD-E75Q/E78A loaded with 0.5 equiv. Eu^{III} and titrated with other Ln^{III} ions. Samples prepared in 20 mM Tris, 100 mM KCl, pH 7.0) buffer. Panel C shows binary separations of light lanthanides using LanD-E75Q/E78A: RT=retentate, FT=flowthrough. The SFs are: SF_{Ce/La}=3.1±0.4. SF_{Pr/La}=5.1±1.3. SF_{Nd/La}=6.9±1.5. Protein (5 μM), 1.7 μM each Ln^{III} ion, in 20 mM MES, 100 mM KCl, pH 6.0. Panel D shows Separations with an equimolar mixture of La^{III}-Nd^{III}. See Table 1 for SFs.

FIG. 8. LanD interacts with apo-LanM. ITC titration of apo-LanD with apo-LanM. Top plot shows raw data for representative titrations of 240 μM apo-Mex-LanM into 30 μM apo-LanD (black) and into buffer (control, red dashes, offset by 75 s for clarity). Bottom plot shows binding isotherm after subtraction of the control titration: the x-axis denotes the molar ratio of Lan M added to LanD. The fit is to an independent model with parameters presented in Table 12. Conditions: 30 mM MOPS, 100 mM KCl, pH 7.0, 25° C.

FIG. 9. The M. extorquens gene cluster for outer-membrane uptake, periplasmic handling, and cytosolic import of lanthanides. This cluster was reported contemporaneously by Ochsner et al, and Mattocks et al, in 2019. The “lan” nomenclature for genes involved in periplasmic lanthanide handling originates with the characterization of lanmodulin (LanM) in 2018 and was followed with LanA for the outer-membrane transporter (functional homolog of META1p1785) in Methylotuvimicrobium buryatense 5GB1C, and by LanP (the lanthanide-binding protein, lanpepsy) in Methylobacillus flagellatus. Two names for META1p1781 have been suggested, LutD and, based on the earlier precedent, LanD. We favor LanD (landiscernin) now that META1p1781 has been biochemically and structurally characterized and its lanthanide ion binding studied in detail. Furthermore, the identification of the interaction of LanD and LanM confirms the connectivity of LanM with the rest of the gene cluster and supports the lan nomenclature for the cluster overall. Adapted with permission from Mattocks et al., copyright 2019 American Chemical Society.

FIG. 10. Size-exclusion chromatograms of apo-LanD during and after purification. Panel A shows data following Q-Sepharose chromatography, protein was loaded onto a calibrated HiLoad 16/600 Superdex S75 column (120 mL) using a 5-mL loop. Apo-LanD elutes as an asymmetrical peak at an apparent molecular weight of 15.5 kDa, suggestive of a dimer trailing into a monomer. Panel B shows data of apoprotein (1.5 mM. 500 μL) was loaded to a calibrated analytical Superdex 75 Increase 10/300 GL column (24-mL volume). Apoprotein elutes as an asymmetric peak at 16.8 kDa. Buffer: 20 mM MES, 100 mM KCl, 5 mM acetate, pH 6.0.

FIG. 11. SEC-MALS trace for apo-LanD illustrating monomer behavior of the apoprotein at lower concentrations. The protein was injected onto the column at a concentration of 250 μM (1.7 mg/mL) and eluted with an apparent molar mass of 7.07 kDa. Buffer: 20 mM Tris, 100 mM KCl, pH 7.0.

FIG. 12. SEC-MALS trace for apo-LanD illustrating dimer behavior of the apoprotein at higher concentrations. The protein was injected onto the column at a concentration of 4.25 mM (28.9 mg/mL) and eluted with an apparent molar mass of 12.8 kDa. Buffer: 20 mM Tris, 100 mM KCl, pH 7.0.

FIG. 13. Size-exclusion chromatogram of purified apo-LanD in buffers with different ionic strengths. Apoprotein (1.5 mM, 500 μL) was loaded onto a calibrated. 24-mL analytical S75 column calibrated. As ionic strength increases, apoprotein elutes later, indicating smaller molecular weights suggestive of dimer dissociation. Samples eluted at volumes corresponding to 16.8 kDa (0.1 M KCl), 14.3 kDa (0.2 M KCl), and 11.2 kDa (0.5 M KCl). Buffers were prepared in 20 mM Tris, pH 7.0.

FIG. 14. Adventitious lanthanide binding sites in LanD samples crystallized at a 1:1 metal:protein ratio. Selected amino acids near the primary metal binding site (La1) and adventitious metal binding sites (La2, La3, and La4) are shown in stick format, La^{III} ions are shown as green spheres, and a coordinated water molecule is shown as a red sphere. An anomalous difference electron density map (green mesh, contoured at 2.8σ) is shown, verifying the assignment of the electron density as either full or partial occupancy La^{III} ions. An inset shows coordination interactions for the adventitious sites, all of which exhibit lower coordination numbers (2-4 versus 8-9 for the primary binding site).

FIG. 15. Disordered side chains in the lanthanide binding site of chain B in the apo-LanD X-ray crystal structure. Chain A (see FIG. 16 below) is fully ordered. Selected sidechains and backbone atoms are shown in stick format with the 2Fₒ-Fₑ (gray mesh, contoured at 1.0σ) and Fₒ-Fₑ difference electron density maps (red/green mesh contoured at +/−3.0σ) shown in overlay.

FIG. 16. Conformational changes in the three metal ligands, E70, E73, and E75, and the second-sphere residue, E78, accompanying La^{III} ion binding and dimer formation. Apo LanD (chain A) is shown in white and La^{III}-LanD is colored orange. Selected amino acid side chains are shown in stick format, the La^{III} ion is shown as a green sphere, and a coordinated water is shown as a red sphere. Coordination interactions are shown as gray dashed lines.

FIG. 17. Views of the 2Fₒ-Fₑ electron density map associated with residue E78, modeled in multiple conformations in all lanthanide-bound LanD X-ray crystal structures. Selected amino acid side chains are shown in stick format, metal ions are shown as colored spheres, water molecules are shown as red spheres, and the 2Fₒ-Fₑ electron density map is shown in colored mesh, contoured at 1.0σ.

FIG. 18. M. extorquens LanD sequence BLAST and sequence alignment. The amino acid sequence for M. extorquens LanD excluding the signal peptide (residues 32-92) was used as the query in a Standard Protein BLAST (NCBI) with an increase in max target sequences to 5000. Of the resulting 263 sequences (51-100% identity with the query sequence), 15 were aligned using a Clustal Omega multiple sequence alignment (UniProt). Signal peptides are in gray, metal ion coordinating residues are bolded in red (E70. E73, and E75 in M. extorquens LanD), and a strictly conserved second coordination sphere residue (E78 in M. extorquens LanD) is bolded in blue. SEQ ID NOs: 19 to 33 are displayed. M, ex.: M. extorquens AMI LanD
1: WP_131002265.1 histidine kinase [Hansschlegelia quercus]
2: HSI42342.1 histidine kinase [Xanthobacteraceae bacterium]
3: WP_343066190.1 histidine kinase [Hansschlegelia beijingensis]
4: WP_201830916.1 hypothetical protein [Microvirga zambiensis]
5: WP_258739177.1 hypothetical protein [Ancylobacter mangrovi]
6: WP_261968080.1 hypothetical protein [Prosthecodimorpha stalevi]
7: WP_247027205.1 hypothetical protein [Ancylobacter crimeensis]
8: WP_149817405.1 hypothetical protein [Salinarimonas soli]
9: WP_213338860.1 histidine kinase [Ancylobacter lacus]
10: MDB5559218.1 hypothetical protein [Enterovirga sp.]
11: HYH17421.1 hypothetical protein [Azospirillum sp.]
12: RXF74813.1 histidine kinase |Hansschlegelia zhihuaiae]
13: PZQ11613.1 MAG: histidine kinase [Ancylobacter novellus]
14: WP_261403330.1 histidine kinase [Chenggangzhangella methanolivorans]
15: WP_309390436.1 histidine kinase [Chelatococcus sambhunathii]

FIG. 19. A sequence conservation map based on an alignment of the 263 LanD orthologs described in FIG. 18 shows conservation of the residues involved in the disulfide bond, the metal binding residues, and a series of charged second-sphere side chains. The other half of the metal-bound dimer is omitted for clarity. This analysis shows that most strictly conserved residues are found in α2 and α3 near the lanthanide binding site. This conservation pattern supports a metal-binding function for the entire group of LanD proteins. Strict conservation of the second-sphere negatively charged residues in α3 also supports the proposal that self-dimerization of LanD may be generally disfavored in vivo. Conservation of these side chains could instead be important for recognition of an exogenous ligand, such as another protein. The most variable region of the LanD proteins is the signal peptide, which is not shown.

FIG. 20. Dimer dissociation of apo-LanD followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 800 μM protein into buffer. (Bottom) Thermogram derived from the data above fitted to the dimer dissociation model using the NanoAnalyze software, with parameters presented in Table 5. The fitted heat from Panel A was subtracted from each titration. Panel C shows overlay of the derived thermograms of three replicates. Conditions: 30 mM MOPS, 100 mM KCl, pH 7.0, 25° C.

FIG. 21. Dimer dissociation of apo-D39S followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 800 μM protein into buffer. (Bottom) Thermogram derived from the data above fitted to the dimer dissociation model using the NanoAnalyze software, with parameters presented in Table 5. The fitted heat from Panel A was subtracted from each titration. Panel C shows overlay of the derived thermograms of three replicates. Conditions: 30 mM MOPS, 100 mM KCl, pH 7.0, 25° C.

FIG. 22. Dimer dissociation of apo-R47K followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 800 μM protein into buffer. (Bottom) Thermogram derived from the data above fitted to the dimer dissociation model using the NanoAnalyze software, with parameters presented in Table 5. The fitted heat from Panel A was subtracted from each titration. Panel C shows overlay of the derived thermograms of three replicates. Conditions: 30 mM MOPS, 100 mM KCl, pH 7.0, 25° C.

FIG. 23. Dimer dissociation of La^{III}-LanD followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 800 μM protein loaded with 0.5 equiv. La^{III} into buffer. (Bottom) Thermogram derived from the data above fitted to the dimer dissociation model using the NanoAnalyze software, with parameters presented in Table 6. The fitted heat from Panel A was subtracted from each titration. Panel C shows overlay of the derived thermograms of four replicates. Conditions: 30 mM MOPS, 100 mM KCl, pH 7.0, 25° C.

FIG. 24. Dimer dissociation of Ce^{III}-LanD followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 800 μM protein loaded with 0.5 equiv. Ce^{III} into buffer. (Bottom) Thermogram derived from the data above fitted to the dimer dissociation model using the NanoAnalyze software, with parameters presented in Table 6. The fitted heat from Panel A was subtracted from each titration. Panel C shows overlay of the derived thermograms of three replicates. Conditions: 30 mM MOPS, 100 mM KCl, pH 7.0, 25° C.

FIG. 25. Dimer dissociation of Nd^{III}-LanD followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 1.0 mM protein loaded with 0.5 equiv. Nd^{III} into buffer. (Bottom) Thermogram derived from the data above fitted to the dimer dissociation model using the Nano Analyze software, with parameters presented in Table 6. The fitted heat from Panel A was subtracted from each titration. Panel C shows overlay of the derived thermograms of three replicates. Conditions: 30 mM MOPS. 100 mM KCl, pH 7.0, 25° C.

FIG. 26. Dimer dissociation of Eu^{III}-LanD followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 1.0 mM protein loaded with 0.5 equiv. Eu^{III} into buffer. (Bottom) Thermogram derived from the data above fitted to the dimer dissociation model using the NanoAnalyze software, with parameters presented in Table 6. The fitted heat from Panel A was subtracted from each titration. Panel C shows overlay of the derived thermograms of three replicates. Conditions: 30 mM MOPS. 100 mM KCl, pH 7.0, 25° C.

FIG. 27. Dimer dissociation of Ho^{III}-LanD followed by ITC. Panel A shows (Top) Representative ITC trace for titration of buffer into buffer. (Bottom) Thermogram derived from the data above fitted to the blank constant model using the Nano Analyze software. Panel B shows (Top) Representative ITC trace for titration of 1.0 mM protein loaded with 0.5 equiv. Ho^{III} into buffer. (Bottom) Thermogram derived from the data above fitted to the dimer dissociation model using the Nano Analyze software, with parameters presented in Table 6. The fitted heat from Panel A was subtracted from each titration. Panel C shows overlay of the derived thermograms of three replicates. Conditions: 30 mM MOPS. 100 mM KCl, pH 7.0, 25° C.

FIG. 28. Views of the electron density maps associated with the first coordination sphere and coordinated water molecule modeled in (Panel A and Panel C La^{III} and (Panel B and Panel D) Ce^{III}-bound LanD X-ray crystal structures. A 2Fₒ-Fₑ electron density map is shown in gray mesh, contoured at 1.5σ. (Panel C and D) In panels C and D, an omit electron density map for the coordinated water is shown in green mesh contoured at 3.0σ.

FIG. 29. Titrations of 5 μM xylenol orange with Ln^{III} ions (Ln=La, Nd, and Eu) in the presence and absence of 10 μM LanD. Ln^{III} ion equivalents are reported on a per monomer basis. The “no protein” trace is for a control experiment with additions of La^{III} to 5 μM xylenol orange in the absence of protein. The stoichiometry suggests a monomer under these experimental conditions. The binding equivalents are slightly higher than those reported previously (˜0.7) (Mattocks et al., JACS 2019), which likely reflects more accurate metal stock solution concentrations (verified by ICP-MS herein.

FIG. 30. Eu^{III}-LanD K_{d1} determination by ITC. Panel A shows (Top) representative ITC trace for a blank titration of 120 μM Eu^{III} into buffer. (Bottom) Thermogram derived from the data above fitted to the blank constant model using the NanoAnalyze software. Panel B shows (Top) representative ITC trace for titration of 120 μM Eu^{III} into 15 μM protein. (Bottom) Thermogram derived from the data above fitted to the independent model using the NanoAnalyze software, with parameters presented in Table 7. The fitted heat from Panel A was subtracted from each titration. Panel C shows overlay of the derived thermograms of three replicates. Conditions: 30 mM MOPS, 100 mM KCl, pH 7.0, 25° C.

FIG. 31. Relative K_{d1} measurements for LanD measured via competitive time-resolved Eu^{III} fluorescence for La^{III}, Ce^{III}, Pr^{III}, Nd^{III}, Sm^{III}, and Gd^{III} (representative data from one of three replicates). The Eu^{III}-based emission of 1:1 mixtures of a Ln^{III} and Eu^{III} (5 μM LanD incubated with 20 μM Ln_{III}, 20 μM Eu^{III}; Ln=La, Ce, Pr, Nd, Sm, or Gd) samples were compared to control samples of 20 μM Eu^{III} in buffer (0% bound) and 5 μM LanD incubated with 20 μM Eu^{III} (100% bound). Displacement of Eu^{III} was monitored by the change in emission at 617 nm and a relative K_{d1} was calculated (see Methods. Table 8, and FIG. 6B). For samples containing Nd^{III}, there was interference of Eu^{III}-based emission. To correct for this interference, a 0% bound sample containing 20 μM Eu^{III} and 20 μM Nd^{III} was measured. The ratio of the area under the two gaussian peaks was determined between the 0% bound control with and without Nd^{III}. The Nd:Eu sample emission spectra were multiplied by this ratio to obtain corrected emission spectra.

FIG. 32. Schematic depicting the rationale for LanD engineering for separations. Panel A shows an enlarged view of the amino acids targeted for mutagenesis to modulate LanD dimerization and metal ion affinities. Panel B shows monomeric wt LanD binds Ln^{III} ions in a 1:1 ratio with a high nM to low μM affinity, which then dimerizes in the high μM to low mM range. Panel C shows the E75Q mutation was postulated to slightly weaken metal binding to the monomer due to its reduced negative charge. Panel D shows the E78A mutation was postulated to eliminate a potential electrostatic and steric clash across the dimer interface, increasing the affinity of the Ln^{III}-dependent dimer.

FIG. 33. (Left) Xylenol orange competition assay for LanD-E75Q. The curves deviate from the minimum value almost immediately, suggesting ability to outcompete XO, but only weakly (note that the dissociation constants of Ln^{III} ions for XO are 1-10 μM). (Right) Luminescence titration of 50 μM LanD-E75Q with Eu^{III} showing 1 equiv, binding, and therefore likely a monomer under these conditions.

FIG. 34. Eu^{III}-LanD-E75Q K_{d1} determination by ITC. Panel A shows (Top) representative ITC trace for a blank titration of 160 μM Eu^{III} into buffer. (Bottom) Thermogram derived from the data above fitted to the blank constant model using the NanoAnalyze software. Panel B shows (Top) representative ITC trace for titration of 160 μM Eu^{III} into 20 μM protein. (Bottom) Thermogram derived from the data above fitted to the independent model using the NanoAnalyze software, with parameters presented in Table 9. The fitted heat from Panel A was subtracted from each titration. Panel C shows overlay of the derived thermograms of three replicates. Conditions: 30 mM MOPS, 100 mM KCl, pH 7.0, 25° C.

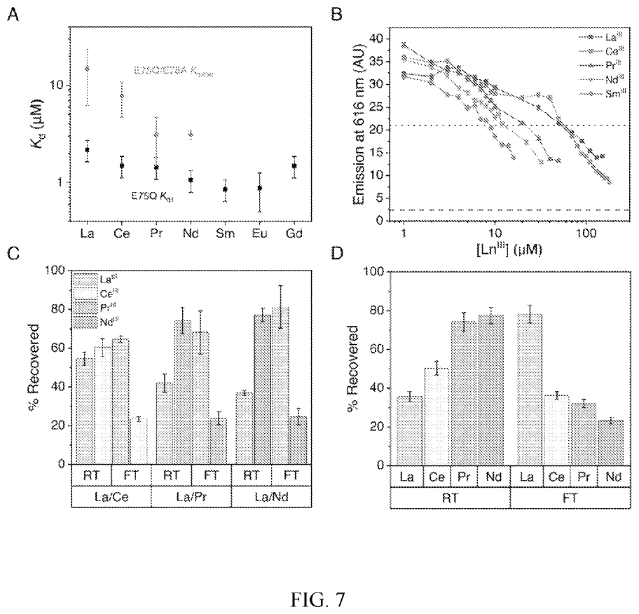

FIG. 35. Relative K_{d1} measurements for LanD-E75Q measured via competitive time-resolved Eu^{III} fluorescence for La^{III}, Ce^{III}, Pr^{III}, Nd^{III}, Sm^{III} and Gd^{III}. The Eu^{III}-based emission of 1:1 mixtures of a Ln^{III} and Eu^{III} (50 μM LanD-E75Q incubated with 200 μM Ln, 200 μM Eu; Ln=La, Ce, Pr, Nd, Sm, or Gd) samples were compared to control samples of 200 μM Eu^{III} in buffer (0% bound) and 50 μM LanD-E75Q incubated with 200 μM Eu^{III} (100% bound). Displacement of Eu^{III} was monitored by the change in emission at 617 nm and a relative K_{d1} was calculated (see Methods and FIG. 7A). For samples containing Pr^{III} and Nd^{III}, there was interference on Eu^{III}-based emission. To correct for this interference, a 0% bound sample containing 200 μM Eu^{III} and 200 μM Pr^{III} or Nd^{III} was measured. The ratio of the area under the two gaussian peaks was determined between the 0% bound control with and without Pr^{III} or Nd^{III}. The Pr:Eu and Nd:Eu sample emission spectra were multiplied by these ratios to obtain corrected emission spectra.

FIG. 36. (Left) Xylenol orange competition assay for LanD-E75Q/E78A (10) μM). The curves suggest K_{dimer} values on the order of the protein concentration. Ln^{III}-xylenol orange K_{d} values are also in this range, which may explain why the binding transition is not sharp; nevertheless, the increase intersects the x-axis in 0.3-0.5 range. (Right) Luminescence titration of 10 μM LanD-E75Q/E78A with Eu^{III} showing ˜0.5 equiv, binding, suggesting the protein is largely a dimer under these conditions.

FIG. 37. Dimer dissociation of La^{III}-E75Q/E78A followed by ITC. Panel A shows (Top) Representative ITC trace for titration of buffer into buffer. (Bottom) Thermogram derived from the data above fitted to the blank constant model using the NanoAnalyze software. Panel B shows (Top) representative ITC trace for titration of 250 μM protein loaded with 0.5 equiv. La^{III} into buffer. (Bottom) Thermogram derived from the data above fitted to the dimer dissociation model using the NanoAnalyze software, with parameters presented in Table 10. The fitted heat from Panel A was subtracted from each titration. Panel C shows overlay of the derived thermograms of three replicates. Conditions: 30 mM MOPS, 100 mM KCl, pH 7.0, 25° C.

FIG. 38. Dimer dissociation of Ce^{III}-E75Q/E78A followed by ITC. Panel A shows (Top) representative ITC trace for titration of buffer into buffer. (Bottom) Thermogram derived from the data above fitted to the blank constant model using the NanoAnalyze software. Panel B shows (Top) representative ITC trace for titration of 150 μM protein loaded with 0.5 equiv. Ce^{III} into buffer. (Bottom) Thermogram derived from the data above fitted to the dimer dissociation model using the NanoAnalyze software, with parameters presented in Table 10. The fitted heat from Panel A was subtracted from each titration. Panel C shows overlay of the derived thermograms of three replicates. Conditions: 30 mM MOPS, 100 mM KCl, pH 7.0, 25° C.

FIG. 39. Dimer dissociation of Pr^{III}-E75Q/E78A followed by ITC. Panel A shows (Top) representative ITC trace for titration of buffer into buffer. (Bottom) Thermogram derived from the data above fitted to the blank constant model using the NanoAnalyze software. Panel B shows (Top) representative ITC trace for titration of 150 μM protein loaded with 0.5 equiv. Pr^{III} into buffer. (Bottom) Thermogram derived from the data above fitted to the dimer dissociation model using the NanoAnalyze software, with parameters presented in Table 10. The fitted heat from Panel A was subtracted from each titration. Panel C shows overlay of the derived thermograms of three replicates. Conditions: 30 mM MOPS, 100 mM KCl, pH 7.0, 25° C.

FIG. 40). Dimer dissociation of Nd^{III}-E75Q/E78A followed by ITC. Panel A shows (Top) representative ITC trace for titration of buffer into buffer. (Bottom) Thermogram derived from the data above fitted to the blank constant model using the NanoAnalyze software. Panel B shows (Top) representative ITC trace for titration of 150 μM protein loaded with 0.5 equiv. Nd^{III} into buffer. (Bottom) Thermogram derived from the data above fitted to the dimer dissociation model using the NanoAnalyze software, with parameters presented in Table 10. The fitted heat from Panel A was subtracted from each titration. Panel C shows overlay of the derived thermograms of three replicates. Conditions: 30 mM MOPS, 100 mM KCl, pH 7.0, 25° C.

FIG. 41. Separation experiment with 12 μM LanD-E75Q/E78A with 4 μM La^{III} and 4 μM Pr^{III} ions, 10 mL. Buffers: 20 mM acetic acid, 100 mM KCl, pH 5.0 and 20 mM MES, 100 mM KCl, pH 6.0. The separation factors determined are SF_{Pr/La}=3.4 at pH 5 and SF_{Pr/La}=4.6 at pH 6. The 3:1 monomer: target metal (Pr or Nd) ratio yielded the best SFs.

FIG. 42. Separation experiment with 5 μM LanM-E75Q/E78A with 2.5 μM La^{III} and 2.5 M Pr^{III} ions, 10 mL, in 20 mM MES, 100 mM KCl, pH 6.0. SF_{Pr/La}=4.4±0.6.

FIG. 43. Separation experiments with wild-type LanD. (Left) 5 μM LanD with 5 μM each La^{III} and Ce^{III}, 10 mL, in 20 mM MES, 100 mM KCl, pH 6.0. SF_{Ce/La}=1.6. (Right) Concentrations were decreased in an attempt to decrease binding of La^{III}: 1 μM LanD with 1 μM each La^{III} and Pr^{III}, pH 6.0, 10 mL. SF_{Pr/La}=1.4.

FIG. 44. Determination of q values for Eu^{III}-LanD in the presence and absence of 200 μM citrate (Panel A) and in the presence of 200 μM 4-hydroxy benzamide or 200 μM 3.4-dihydroxy benzamide (Panel B). The LanM sample (known to have two coordinated solvent molecules per site) was prepared as a control. (Panel C) Structures of methylolanthanin and rhodopetrobactin, with their key potential metal-chelating moieties indicated in red, blue, and green, mimicked herein by citrate, 3-hydroxy benzamide, and 3,4-dihydroxy benzamide, respectively. (Panel D) Graph of q values for Eu^{III}-LanD (20 μM LanD, 18 μM Eu^{III}) alone (black) and in the presence of 200 μM citrate (red), 4-hydroxy benzamide (blue), or 3.4-dihydroxy benzamide (green), q values have intrinsic uncertainties of ±0.5. The slightly higher q value for LanD with 0.9 equiv. Eu^{III} (4.8) than with 0.5 equiv. (4.1, FIG. 6A) may reflect this uncertainty. Ternary complex formation would be expected to decrease the q value for Eu^{III}, which was not observed. Furthermore, the q value in the presence of LanD and 200 μM citrate was lower than for Eu^{III} in the presence of 200 μM citrate in the absence of LanD (q=6, panel A), indicating that citrate does not outcompete LanD for Eu^{III} under these conditions.

FIG. 45. Steady-state luminescence titration of citrate into 15 μM Eu-LanD. Representative titration of citrate into Eu-LanD depicting the titration from 0 mM to 0.2 mM added citrate (top left) and from 0.2 mM to 2 mM added citrate (top right). A control sample of 2 mM citrate with 15 μM Eu^{III} is depicted to compare emission intensity to complete outcompetition of protein binding to Eu^{III} by citrate. The average emission intensity at 616 nm with standard deviation error bars of two replicates is depicted (bottom center). All titrations were conducted in Chelex-treated 20 mM Tris. 100 mM KCl, pH 7.0 buffer. The baseline was corrected by fitting to a high order polynomial (see Methods).

FIG. 46. Time-resolved luminescence titration of Eu^{III}-LanD with 3.4-dihydroxy benzamide (3,4-DHBA). Titration of 3,4-DHBA into a solution containing 15 μM Eu^{III}-LanD (left). Control samples of 10.0 mM 3,4-DHBA with 15 μM Eu^{III}, and of Eu^{III} alone, are shown to compare emission intensity of samples to outcompetition of protein binding to Eu^{III} by 3,4-DHBA. The plot of emission intensity at 617 nm (right) shows an apparent K_{d} of 1-2 mM. Catechols are known to be robust ligands for lanthanides (log K_{ML} for catechol binding to Eu^{III} is 10) and quenching of Eu^{III} luminescence by 3.4-dihydroxy benzoic acid has been previously observed. All titrations conducted in Chelex-treated 30 mM MOPS, 100 mM KCl, pH 7.0 buffer. Spectra were fitted to two Gaussian peaks (see Methods).

FIG. 47. Similar Ln^{III} affinity trend for monomeric LanD and LanM. Plot of K_{d1} values for Ln^{III}-LanD (black squares). These values are compared to −log (D) values for LanM, representing inter-REE selectivity of the protein in batch separation experiments (gray circles), reproduced from Mattocks et al. (Nature 2023).

FIG. 48. La^{III} transfer to LaMP1 is not kinetically limited. LanD (10 μM) or LaMP1 (1 μM) was pre-incubated with 4 μM La^{III} for 20 min and apo-LaMP1 (1 μM) or apo-LanD (10 μM), respectively, was added. The FRET ratio of LaMP1 (λₑₓ=433 nm) was monitored every 5 min for 1 h (the first point was taken ˜30 s after mixing). (Red circles) La^{III}-LaMP1 was mixed with apo-LanD. (Blue triangles) La^{III}-LanD was mixed with apo-LaMP1. (Black squares) Apo-LaMP1 and apo-LanD control.

FIG. 49. LanM-LanD K_{d} determination by ITC. Panel A shows (Top) representative ITC trace for a blank titration of 240 μM apo-Mex-LanM into buffer. (Bottom) Thermogram derived from the data above fitted to the blank constant model using the NanoAnalyze software. Panel B shows (Top) representative ITC trace for titration of 240 μM apo-Mex-LanM into 30 μM apo-LanD. (Bottom) Thermogram derived from the data above fitted to the independent model using the NanoAnalyze software, with parameters presented in Table 12. The fitted heat from Panel A was subtracted from each titration. Panel C shows overlay of the derived thermograms of three replicates. Conditions: 30 mM MOPS, 100 mM KCl, pH 7.0, 25° C.

FIG. 50. Titration of Sm₃-Mex-LanM into apo-LanD monitored by ITC. Panel A shows (Top) representative ITC trace for a blank titration of 240 μM Sm₃-Mex-LanM into buffer. (Bottom) Thermogram derived from the data above fitted to the blank constant model using the NanoAnalyze software. Panel B shows (Top) representative ITC trace for titration of 240 μM Sm₃-Mex-LanM into 30 μM apo-LanD. (Bottom) Thermogram derived from the data above. The fitted heat from Panel A was subtracted from each titration. Panel C shows overlay of the derived thermograms of three replicates. Conditions: 30 mM MOPS, 100 mM KCl, pH 7.0, 25° C.

FIG. 51. Titration of apo-Hans-LanM into apo-LanD monitored by ITC. Panel A shows representative ITC trace for titration of 240 μM Hans-LanM into 30 μM apo-LanD (black line) compared to control titration of 240 μM Hans-LanM into buffer (red dashes, offset by 50 s for clarity). The large heats associated with the control titration are a result of the monomer-dimer equilibrium of Hans-LanM (K_{dimer}=117 μM at 30° C.). Panel B shows thermograms derived from three experimental titrations, following subtraction of the control titration of 240 μM Hans-LanM into buffer. Conditions: 30 mM MOPS, 100 mM KCl, pH 7.0, 25° C.

FIG. 52. Comparison of the La^{III} coordination environment in LanD (Panel A) with previously characterized lanthanide-binding sites. The metal binding site of XoxF from Methylomicrobium buryatense 5GB1C (Panel B, PDB code: 6DAM) includes a pyrroloquinoline quinone (PQQ) cofactor. The La^{III} site of EF-hand 3 in Hans-LanM (Panel C. PDB code: 8DQ2) and the Nd^{III} site of EF-hand 3 in Mex-LanM (Panel D. PDB code: 8FNS) are both composed exclusively of protein-derived and water ligands. The XoxF and Hans-LanM La^{III} sites feature four multidentate ligands (three carboxy lates and PQQ in XoxF, and four carboxylates in Hans-LanM). The Mex-LanM Nd^{III} site contains two bidentate carboxylates. In LanD, each protomer provides two bidentate carboxylates. E70 in each chain, along with four other ligands (monodentate E73 and E75 from each protomer) to the bound La^{III}. The arrangement of ligands in the LanD protomer makes it such that all the carboxylate side chains must face each other in the crystallographic dimer, resulting in an excessively negatively charged metal site. By contrast, the EF-hand arrangement in LanMs allows the carboxylates to be arranged such that they do not directly face each other, and it allows for involvement of backbone atoms in completing the coordination sphere. These observations support the conclusion that LanD is unlikely to bind Ln^{III} ions as a dimer under physiological conditions.

FIG. 53. Plot of K_{d1} values for La^{III}-Lu^{III} complexes of wt LanD, determined from luminescence competition with Eu^{III} (extension of data shown in FIG. 6B)

FIG. 54. (Top) Binary separations of light lanthanides using LanD-E75Q/E78A: RT=retentate, FT=flowthrough. Protein (5 μM), 1.7 μM each Ln^{III} ion, in 20 mM MES, 100 mM KCl, pH 6.0. A 10 mL solution was concentrated 20-fold using a 10-kDa MWCO regenerated cellulose filter. (Bottom) Separation factors determined from the data in the top figure.

FIG. 55. Characterization of LanD-E78A. (Panel A) Xylenol orange competition assay with E78A (5 μM XO, 10 μM LanD-E78A, pH 6.0, into which La, Pr, Nd, or Eu were titrated. (Panel B) Under conditions in which LanD-E75Q/E78A is a predominantly a dimer (10 μM protein titrating in Eu^{III}: see FIG. 36), stoichiometric titration with Eu^{III} does not clearly saturate. (Panel C) At 50 μM protein, the titration saturates at ˜0.5 equiv., suggestive of a dimer. Therefore, the LanD-E78A K_{dimer} is weaker than that of LanD-E75Q/E78A. (Panel D) Time-resolved luminescence competition titration with 50 μM LanD-E78A loaded with 0.5 equiv. Eu^{III} and titrated with other Ln^{III} ions. Samples prepared in 20 mM Tris, 100 mM KCl, pH 7.0 buffer.

FIG. 56. Separation experiment with 5 μM LanM-E78A with 1.7 μM La^{III} and 1.7 μM Nd^{III} ions, 10 mL, in 20 mM MES, 100 mM KCl, pH 6.0. SF_{Nd/La}=5.8.

FIG. 57. (Panel A) Xylenol orange titration of LanD-D77N/E81A (5 μM XO, 10 μM protein, pH 6.0). (Panel B) Stoichiometric time-resolved fluorescence titration of 10 μM LanD-D77N/E81A with Eu^{III}.

FIG. 58. (Panel A) Time-resolved luminescence competition titration with 10 μM LanD-D77N/E81A loaded with 1.0 equiv. Eu^{III} and titrated with other Ln^{III} ions. (Panel B) Separation experiment using LanD-D77N/E81A: 1 μM LanD variant with 1 μM each La^{III} and Pr^{III}, 10 mL, in 20 mM MES, 100 mM KCl, pH 6.0.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, process step, and electronic changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%. 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

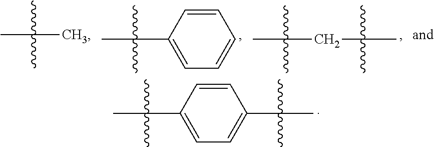

Amino acids and amino acid residues may be referred to herein by either their commonly known three letter symbols or by the one-letter symbols recommended by the IUPAC-IUB Biochemical Nomenclature Commission.

Examples of hydrophobic amino acid and hydrophobic amino acid residues include, but are not limited to, glycine, alanine, valine, leucine, isoleucine, proline, cysteine, phenylalanine, methionine, and tryptophan.

The present disclosure provides proteins that bind rare earth metal ions. Also provided are devices and kits comprising a protein of the present disclosure. Also provided are methods of using the proteins and devices.

In an aspect, the present disclosure provides proteins that bind metal ions (e.g., lanthanide ions and/or actinide ions). Other metal-binding proteins are disclosed in WO2020051274, WO2023004333, and WO2024155330 which are incorporated herein by reference. As used throughout, the term “metal” refers to metal ions.

A protein of the present disclosure may be of various lengths. For example, a protein of the present disclosure has 50 to 175 amino acid residues, including all integer amino acid values and ranges therebetween (e.g., 55 to 150 amino acid residues). For example, the protein has a molecular weight of around 6 kDa to 14 kDa, including all 0.1 Da values and ranges therebetween (e.g., ˜12 kDa). A protein of the present disclosure comprises at least one segment where one or more rare earth metals can bind.

A metal-binding protein of the present disclosure may comprise signal sequence:

(MMRTRTSLAVPRGFRGSALLALVVLATPALADDKAACAD
GIAAVKARVEKLAPEAVPQKLKRALKIAEREQGEGEFDEC
LEALDDAKRALPKYG (SEQ ID NO: 2))

or comprise a sequence that excludes a signal sequence:

(SEQ ID NO: 34)
DDKAACADGIAAVKARVEKLAPEAVPQKLKRALK
IAEREQGEGEFDECLEALDDAKRALPKYG,

where the signal sequence or signal peptide has the following sequence:

(SEQ ID NO: 51)
MMRTRTSLAVPRGFRGSALLALVVLATPALA.

In various examples, a metal-binding protein of the present disclosure has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99%) identity to SEQ ID NO:2 or SEQ ID NO:34. In various examples, a protein of the present disclosure may be affixed or disposed on a substrate. The Signal Peptide may be removed prior to using the protein.

In various examples, a metal-binding protein of the present disclosure has the following sequence:

(SEQ ID NO: 1)
DDKAACAX¹GIAAVKAX²VEKLAPEAVPQKLKRALKI
AEREQGEGX³FX⁴X³CLX⁶ALX⁷DAKRALPKX⁸X⁹
or
(SEQ ID NO: 52)
MMRTRTSLAVPRGFRGSALLADDKAACAX¹GIAAVKA
X²VEKLAPEAVPQKLKRALKIAEREQGEGX³FX⁴X⁵C
LX⁶ALX⁷DAKRALPKX⁸X⁹,

where X¹ is D or S: X² is R or K: X³ is E, Q, or M: X⁴ is D, N, Q, or K; X⁵ is E, N, Q, D, A, or T: X⁶ is E, A, or Q: X⁷ is D or E: X⁸ is Y, W, or absent; and X⁹ is G or absent. In various examples, a metal-binding protein may have at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99%) identity to SEQ ID NO: 1 or SEQ ID NO:52. In various examples, the signal sequence may be removed prior to using the protein.

A metal-binding protein of the present disclosure may have or comprise any one of the following sequences:

| | SEQ |
|---|---|
| | ID |
| Sequence | NO |
| DDKAACAXGI AAVKAXVEKL APEAVPQKLK RALKIAEREQ | 1 |
| GEGXFXXCLX ALXDAKRALP KXX | |
| MMRTRTSLAV PRGFRGSALL ALVVLATPAL ADDKAACADG | 2 |
| IAAVKARVEK LAPEAVPQKL KRALKIAERE QGEGEFDECL | |
| EALDDAKRAL PKYG | |
| MMRTRTSLAV PRGFRGSALL ALVVLATPAL ADDKAACASG | 3 |
| IAAVKARVEK LAPEAVPQKL KRALKIAERE QGEGEFDECL | |
| EALDDAKRAL PKYG | |
| MMRTRTSLAV PRGFRGSALL ALVVLATPAL ADDKAACADG | 4 |
| IAAVKAKVEK LAPEAVPQKL KRALKIAERE QGEGEFDECL | |
| EALDDAKRAL PKYG | |
| MMRTRTSLAV PRGFRGSALL ALVVLATPAL ADDKAACADG | 5 |
| IAAVKARVEK LAPEAVPQKL KRALKIAERE QGEGQFDECL | |
| EALDDAKRAL PKYG | |
| MMRTRTSLAV PRGFRGSALL ALVVLATPAL ADDKAACADG | 6 |
| IAAVKARVEK LAPEAVPQKL KRALKIAERE QGEGEFDACL | |
| EALDDAKRAL PKYG | |
| MMRTRTSLAV PRGFRGSALL ALVVLATPAL ADDKAACADG | 7 |
| IAAVKARVEK LAPEAVPQKL KRALKIAERE QGEGEFDNCL | |
| EALDDAKRAL PKYG | |
| MMRTRTSLAV PRGFRGSALL ALVVLATPAL ADDKAACADG | 8 |
| IAAVKARVEK LAPEAVPQKL KRALKIAERE QGEGEFDQCL | |
| EALDDAKRAL PKYG | |
| MMRTRTSLAV PRGFRGSALL ALVVLATPAL ADDKAACADG | 9 |
| IAAVKARVEK LAPEAVPQKL KRALKIAERE QGEGEFDDCL | |
| EALDDAKRAL PKYG | |
| MMRTRTSLAV PRGFRGSALL ALVVLATPAL ADDKAACADG | 10 |
| IAAVKARVEK LAPEAVPQKI KRALKIAERE QGEGQFDACL | |
| EALDDAKRAL PKYG | |
| MMRTRTSLAV PRGFRGSALL ALVVLATPAL ADDKAACADG | 11 |
| IAAVKARVEK LAPEAVPQKL KRALKIAERE QGEGQFDNCL | |
| EALDDAKRAL PKYG | |
| MMRTRTSLAV PRGFRGSALL ALVVLATPAL ADDKAACADG | 12 |
| IAAVKARVEK LAPEAVPQKL KRALKIAERE QGEGEFNECL | |
| AALDDAKRAL PKYG | |
| MMRTRTSLAV PRGFRGSALL ALVVLATPAL ADDKAACADG | 13 |
| IAAVKARVEK LAPEAVPQKL KRALKIAERE QGEGEFQECL | |
| QALDDAKRAL PKYG | |
| MMRTRTSLAV PRGERGSALL ALVVLATPAL ADDKAACADG | 14 |
| IAAVKARVEK LAPEAVPQKL KRALKIAERE QGEGEFKECL | |
| QALDDAKRAL PKYG | |
| MMRTRTSLAV PRGFRGSALL ALVVLATPAL ADDKAACADG | 15 |
| IAAVKARVEK LAPEAVPQKL KRALKIAERE QGEGQFDTCL | |
| EALDDAKRAL PKYG | |
| MMRTRTSLAV PRGFRGSALL ALVVLATPAL ADDKAACADG | 16 |
| IAAVKARVEK LAPEAVPQKL KRALKIAERE QGEGQFDACL | |
| EALEDAKRAL PKYG | |
| MMRTRTSLAV PRGFRGSALL ALVVLATPAL ADDKAACADG | 17 |
| IAAVKARVEK LAPEAVPQKI KRALKIAERE QGEGMEDACL | |
| EALDDAKRAL PKYG | |
| DDKAACADGI AAVKARVEKL APEAVPQKLK RALKIAEREQ | 34 |
| GEGEFDECLE ALDDAKRALP KYG | |
| DDKAACASGI AAVKARVEKL APEAVPQKLK RALKIAEREQ | 35 |
| GEGEFDECLE ALDDAKRALP KYG | |
| DDKAACADGI AAVKAKVEKL APEAVPQKLK RALKIAEREQ | 36 |
| GEGEFDECLE ALDDAKRALP KYG | |
| DDKAACADGI AAVKARVEKL APEAVPQKLK RALKIAEREQ | 37 |
| GEGQFDECLE ALDDAKRALP KYG | |
| DDKAACADGI AAVKARVEKL APEAVPQKLK RALKIAEREQ | 38 |
| GEGEFDACLE ALDDAKRALP KYG | |
| DDKAACADGI AAVKARVEKL APEAVPQKLK RALKIAEREQ | 38 |
| GEGEFDACLE ALDDAKRALP KYG | |
| DDKAACADGI AAVKARVEKL APEAVPQKLK RALKIAEREQ | 39 |
| GEGEFDNCLE ALDDAKRALP KYG | |
| DDKAACADGI AAVKARVEKL APEAVPQKLK RALKIAEREQ | 40 |
| GEGEFDQCLE ALDDAKRALP KYG | |
| DDKAACADGI AAVKARVEKL APEAVPQKLK RALKIAEREQ | 41 |
| GEGEFDDCLE ALDDAKRALP KYG | |
| DDKAACADGI AAVKARVEKL APEAVPQKLK RALKIAEREQ | 42 |
| GEGQFDACLE ALDDAKRALP KYG | |
| DDKAACADGI AAVKARVEKL APEAVPQKLK RALKIAEREQ | 43 |
| GEGQFDNCLE ALDDAKRALP KYG | |
| DDKAACADGI AAVKARVEKL APEAVPQKLK RALKIAEREQ | 44 |
| GEGEFNECLA ALDDAKRALP KYG | |
| DDKAACADGI AAVKARVEKL APEAVPQKLK RALKIAEREQ | 45 |
| GEGEFQECLQ ALDDAKRALP KYG | |
| DDKAACADGI AAVKARVEKL APEAVPQKLK RALKIAEREQ | 46 |
| GEGEFKECLQ ALDDAKRALP KYG | |
| DDKAACADGI AAVKARVEKL APEAVPQKLK RALKIAEREQ | 47 |
| GEGQFDTCLE ALDDAKRALP KYG | |
| DDKAACADGI AAVKARVEKL APEAVPQKLK RALKIAEREQ | 48 |
| GEGQFDACLE ALEDAKRALP KYG | |
| DDKAACADGI AAVKARVEKL APEAVPQKLK RALKIAEREQ | 49 |
| GEGMFDACLE ALDDAKRALP KYG | |
| DDKAACADGI AAVKARVEKL APEAVPQKLK RALKIAEREQ | 50 |
| GEGEFDECLE ALDDAKRALP K | |
| MMRTRTSLAV PRGFRGSALL ALVVLATPAL ADDKAACAXG | 52 |
| IAAVKAXVEK LAPEAVPQKL KRALKIAERE QGEGXFXXCL | |
| XALXDAKRAL PKXX | |

In various examples, a metal-binding protein comprises one or more metal-binding or metal coordination motifs. The one or more metal-binding or metal coordination motifs may have the following sequence: REX¹X²EX³EX⁴DEC (SEQ ID NO:53), where X¹ is any amino acid; X² is any amino acid, (e.g., G, A, K, R); X³ is any amino acid, (e.g., G, A, or K); and X⁴ is F or Y; and the C forms a disulfide bond with another cysteine residue elsewhere in the polypeptide. In various examples, any one or more of the non-X residues may be substituted with an amino acid that is isoelectronic, isostructural, or be replaced with alanine. For example, any D can be replaced with N, E, or A; any E can be replaced with Q. D, or A; or R can be replaced with K. In various examples, any one or more of the glutamic acid residues may be replaced with a softer Lewis base amino acid residue (e.g., C, M, Q, or H) (e.g., RX⁵X¹X²X⁶X³X⁷X⁴DX⁸C, where X⁵, X⁶, X⁷, and X⁸ are independently chosen from E, C, M, Q, or H.

The metal-binding protein of the present disclosure may further be concatenated with the same or different metal-binding protein of the present disclosure. As an illustrative example, DDKAACADGIAAVKARVEKLAPEAVPQKLKRALKIAEREQGEGQFDACLEALDDA KRALPKYG (SEQ ID NO:34) can be concatenated with another strand of SEQ ID NO:34 or any other protein of the present disclosure. In various examples, any two of SEQ ID NO: 1 and 34-50, and each sequence could be the same or different. The two sequences can be concatenated by a linking group. In various examples, the linking group is a peptide comprising 5 to 30 amino acid residues. In various other examples, the linking group is an aliphatic group or a poly(ethylene)glycol group or other suitable carbon-based linking groups

In various examples, a metal-binding protein of the present disclosure is affixed or disposed on a substrate. Various substrates may be used. Non-limiting examples of substrates include, but are not limited to, a bead (e.g., agarose, silica, polymeric resin, or the like), a membrane, a hydrogel, a protein-based material, a porous framework (e.g., MOF), and others known in the art.

In various examples, a protein of the present disclosure has a residue suitable for immobilization onto the substrate. The residue may be part of a large sequence comprising 2 to 10 amino acid residues. For example, the residue comprises a functional group that chemically reacts with another functional group on the substrate such that the residue (and thus protein) is covalently attached to the substrate. For example, the substrate may comprise a maleimide group or a succinimide group that can react with a nucleophilic group, such as the thiol of a cysteine or amine of a lysine or ornithine or a nucleophilic atom of a non-canonical amino acid. Other suitable chemistries (e.g., Click chemistry, Spy Tag/Spy Catcher, and the like) are known in the art and may be used. For example, the substrate may be a resin or bead comprising a functional group that can react with the residue of the metal-binding protein. For example, the functional group may be a maleimide, alkyne, or azide.

In an aspect, the present disclosure provides various methods of using the proteins and/or devices of the present disclosure. A method of the present disclosure may be for binding one or more lanthanides and/or actinides or for detecting and/or quantifying the amount of one or more lanthanides and/or actinides.

A method of using a protein and/or device of the present disclosure may be a method for binding one or more rare earth metals (e.g., lanthanides and/or actinides) in a sample. Binding may occur by contacting the sample with one or more proteins and/or devices of the present disclosure. The method may be performed on various types of samples. Examples of samples include, but are not limited to drinking water, wastewater, ground water, ash ponds, aqueous extract from contaminated soil, drainage (e.g., mine drainage, such as, for example, acidic mine drainage) or leachate (e.g., electronic waste leachate or leachate of an ore leachate). In various other examples, the sample is a solid sample. The method may be applied to samples over a variety of pH values (e.g., 4 to 8, including all 0.01 pH values and ranges therebetween).

Various lanthanides (e.g., lanthanide ions) and/or actinides (e.g., actinide ions) may be bound by a protein and/or device. Examples of lanthanide ions and actinide ions that may be bound include, but are not limited to, La, Ce, Pr, Nd, Pm, Sm, Eu, Gd, Tb, Dy, Ho, Er, Tm, Yb, Lu, Sc, Y, Ac, Th, U, Np, Pu, Am, Cm, Bk, Cf and various ions thereof. In various examples, any lanthanide ion or any actinide ion is bound. For example, the lanthanide is chosen from Pr, Nd, Sm, Eu, Gd, Tm, Yb, and Lu, and ions thereof. In various examples, the lanthanide is Pr, Nd, Sm, or an ion thereof: or Tm, Yb, Lu, or an ion thereof: or Am, Cm, or an ion thereof. The concentration of the lanthanide and/or actinides in the sample may be less than 100 ppm (e.g., less than 90, 80, 70, 60, 50, 40, 30, 20, 10, 1, 0.1, or 0.05 ppm).

In various examples, the one or more lanthanides and/or actinides bound to the one or more proteins and/or devices may be isolated from the proteins and/or devices and recovered. The lanthanides and/or actinides may be unbound by lowering the pH below ˜4 or by adding a chelator (e.g., citrate, EDTA, EGTA, malonate, or the like). In various embodiments, if one or more different lanthanides and/or actinides are bound to the one or more proteins or devices, the one or more different lanthanides and/or actinides may be sequentially dissociated from the proteins. As an illustrative example, if both La and Nd are bound, one species of metal can be selectively dissociated, while the other metal remains bound. For example, one metal can be dissociated via contacting with a chelator, while the other metal is dissociated via adjustment of the pH. The one or more proteins and/or devices may be reused after the one or more lanthanides are unbound and separated.

Various lanthanides (e.g., lanthanide ions) and/or actinides (e.g., actinide ions) may be bound by a protein and/or device. For example, the lanthanide ion is any lanthanide ion, or the actinide ion is any actinide ion. The bound lanthanides and/or actinides may be the same or different. The concentration of the lanthanide and/or actinide in the sample may be less than 1 ppm.

In an aspect, the present disclosure provides kits. The kits may comprise a protein of the present disclosure. The kit may further comprise instructions for use. Additionally, a kit may comprise a substate, instructions and materials to conjugate or otherwise attach the protein to the substrate.

In an aspect, the present disclosure provides methods of making a protein of the present disclosure. A protein may be made by methods known in the art, such as by ligation, solid phase peptide synthesis (SPPS), or expression in a bacterial cell.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following sequences used throughout the disclosure, where the X's are as defined above:

| | | SEQ |
|---|---|---|
| | | ID |
| Name | Sequence | NO |
| LanD-Mutant w/o | DDKAACAXGI AAVKAXVEKL APEAVPQKLK RALKIAEREQ GEGXEXXCLA ALXDAKRALP | 1 |
| Signal peptide | KXX | |
| WT-LanD | MMRTRTSLAV PRGFRGSALL ALVVLATPAL ADDKAACADG IAAVKARVEK LAPEAVPQKL | 2 |
| | KRASKIAERE QGEGEFDECL EALDDAKRAL PKYG | |
| LanD-D39S | MMRTRTSLAV PRGFRGSALL ALVVLATPAL ADDKAACA [symbol] G IAAVKARVEK LAPEAVPQKL | 3 |
| | KRALKIAERE QGEGEFDECL EALDDAKRAL PKYG | |
| LanD-R47K | MMRTRTSLAV PRGFRGSALL ALVVLATPAL ADDKAACADG IAAVKA [symbol] VEK LAPEAVPQKL | 4 |
| | KRALKIAERE QGEGEFDECL EALDDAKRAL PKYG | |
| LanD-E75Q | MMRTRTSLAV PRGFRGSALL ALVVLATPAL ADDKAACADG IAAVKARVEK LAPEAVPQKL | 5 |
| | KRALKIAERE QGEGEDE [symbol] CL EALDDAKRAL PKYG | |
| TanD-F78A | MMRTRTSLAV PRGFRGSALL ALVVLATPAL ADDKAACADG IAAVKARVEK LAPEAVPQKL | 5 |
| | KRALKIAERE QGEGEFD [symbol] CL EALDDAKRAL PKYG | |
| TanD-F78N | MMRTRTSLAV PRGFRGSALL ALVVLATPAL ADDKAACADG IAAVKARVEK LAPEAVPQKL | 7 |
| | KRALKIAERE QGEGEFD [symbol] CL EALDDAKRAL PKYG | |
| TanD-F78Q | MMRTRTSLAV PRGFRGSALL ALVVLATPAL ADDKAACADG IAAVKARVEK LAPEAVPQKL | 8 |
| | KRALKIAERE QGEGEFD [symbol] CL EALDDAKRAL PKYG | |
| TanD-F78D | MMRTRTSLAV PRGFRGSALL ALVVLATPAL ADDKAACADG IAAVKARVEK LAPEAVPQKL | 9 |
| | KRALKIAERE QGEGEFD [symbol] CL EALDDAKRAL PKYG | |
| TanD-E75Q/F78A | MMRTRTSLAV PRGFRGSALL ALVVLATPAL ADDKAACADG IAAVKARVEK LAPEAVPQKL | 10 |
| | KRALKIAERE QGEG [symbol] FD [symbol] CL EALDDAKRAL PKYG | |
| TanD-E75Q/F78N | MMRTRTSLAV PRGFRGSALL ALVVLATPAL ADDKAACADG IAAVKARVEK LAPEAVPQKL | 11 |
| | KRALKIAERE QGEG [symbol] FD [symbol] CL EALDDAKRAL PKYG | |
| LanD-D77N/E81A | MMRTRTSLAV PRGFRGSALL ALVVLATPAL ADDKAACADG IAAVKARVEK LAPEAVPQKL | 12 |
| | KRALKIAERE QGEGEF [symbol] ECL [symbol] ALDDAKRAL PKYG | |
| LanD-D77Q/E81Q | MMRTRTSLAV PRGFRGSALL ALVVLATPAL ADDKAACADG IAAVKARVEK LAPEAVPQKL | 13 |
| | KRALKIAERE QGEGEF [symbol] ECL [symbol] ALDDAKRAL PKYG | |
| LanD-D77K/E81Q | MMRTRTSLAV PRGFRGSALL ALVVLATPAL ADDKAACADG IAAVKARVEK LAPEAVPQKL | 14 |
| | KRALKIAERE QGEGEF [symbol] ECL [symbol] ALDDAKRAL PKYG | |
| LanD-E75Q/E78T | MMRTRTSLAV PRGFRGSALL ALVVLATPAL ADDKAACADG IAAVKARVEK LAPEAVPQKL | 15 |
| | KRALKIAERE QGEG [symbol] FD [symbol] CL EALDDAKRAL PKYG | |
| LanD- | MMRTRTSLAV PRGFRGSALL ALVVLATPAL ADDKAACADG IAAVKARVEK LAPEAVPQKL | 16 |
| E75Q/E78A/D84E | KRALKIAERE QGEG [symbol] FD [symbol] CL EAL [symbol] DAKRAL PKYG | |
| LanD-E75M/E78A | MMRTRTSLAV PRGFRGSALL ALVVLATPAL ADDKAACADG IAAVKARVEK LAPEAVPQKL | 17 |
| | KRALKIAERE QGEGMEDACL EALDDAKRAL PKYG | |
| M. extorquens AM1 | MMRTRTSLAV PRGFRGSALL ALVVLATPAL ADDKAACADG IAAVKARVEK LAPEAVPQKL | 18 |
| | KRALKIAERE QGEGEFDECL EALDDAKRAL PK | |
| Hansschlegelia | MRIYLLASLA LLATLFAARA DDAADCDAGI AMIRMEQAKE HGRATTESLK TALRVAEREK | 19 |
| quercus | GEKEYDECLD AVADARKALK K | |
| Xanthobacteraceae | MNQZNAALLA ATLIGVSASA VRADDAKVCT DGIAMIKAEI AKTEPKATLD KLNKALKGAE | 20 |
| bacterium | REYGEKEFDE CVDFVNDAKK AKG | |
| Hansschlegelia | MMKVFLLAAA GLAFAGATAQ ADEASDCDAG IAMIRAEAAG SHPFAVADSL KTALRVAERE | 21 |
| beijingensis | QGEKEYDECL DAIDDAKKAL NKK | |
| Microvirga | MRPALMSCLV LITFASSVMP AVADDQADCT AGIAMIKAEL DKKEPQTTLT ALQRALRSAE | 22 |
| zambiensis | RELKEAEFDE CVDAVIDAKK ALGR | |
| Ancylobacter | MRERTLRILL SAALLAGAAT FATLGSALAD DAKDCSDGVA MIKAEIAKGP PKATLDKLNK | 23 |
| mangrovi | ALRGAQREMG EGEYDECLDF VGDAKKAIKG | |
| Prosthecodimorpha | MLRPALVVAA LLTASGPALA ADDLASCTKG TAFTKAETAK NPPAPVLTRL KKALKDANRE | 24 |
| staleyi | LGEGEFDECM DAVRDAEKTT GRKS | |
| Ancylobacter | MRYTGLKLAL GLAAGLAFGG VALADDAKTC NDGIAMIKAE IAKKPPKATL DKLTKALKGA | 25 |
| crimeensis | EREHGEKAYD ECVDYTKDAQ KAVGG | |
| Salinarimonas soli | MRRTVVPMIG LAAATAAPVP ALADDQADCV AGTAMTRAET AKNFPQATIT ALQRALRSAE | 26 |
| | RETKEAEFDE CVDAVNDARK ALRR | |
| Ancylobacter lacus | MTRTLATAAA VLVLGIGIAA FADDAKQCSD GITELKAEAA KNPFKATLDK IKKALKGAER | 27 |
| | EGGEKEYDES IDYVNDGKKA VSG | |
| Enterovirga sp. | MGRILLSVSL VIAAGKIGGE PASADDRSEC AAGIAMLEAE LSKATGAVRT KVERELRVAR | 28 |
| | REQAEGEFDE CMDATRAARP ALRQ | |
| Azospirillum sp. | MRTPTFAVLT ATLLATPALA DDQSECVAGI GFIKAEIAKA PPQPILDALK KQLRNAEREQ | 29 |
| | REKEYDECID AVTAARKAVA AK | |
| Hansschlegelia | MLALGLVAAA ALACPEARAD AAADCDSGIA MITAESAKEQ PGPVADALKT ALRVAKREKG | 30 |
| chihuaiae | EQEYDECIDA VEDARKATKK K | |
| Ancylobacter | MTGLRTFAAS LAFLSACVVA AGSARADDAA DCDAGIEMIT AEIAGEHPKA TADALRTALR | 31 |
| novellus | VAKREKGEKE YDECLDAVAD AKKALRK | |
| Chenggangchangella | MSARTHSALT GLALLAALAL PAPAFADDAA DCDAGIAMIS SEVAKEHPKA TADALKTALR | 32 |
| methanolivorans | VAKREKGEKE YDECLDAVAD AKKALGR | |
| Chelatococcus | MSALQHSFRA LTIAAALSVS APAFADAAAD CDAGIEMISA EVAKEEPKAA AEALKKALKV | 33 |
| sambhunathii | AKREKGEKEY DECLDAVADA KKALGR | |
| WT-LanD w/o signal | DDKAACADGI AAVKARVEKL APEAVPQKLK RALKIAEREQ GEGEFDECLE ALDDAKRALP | 34 |
| peptide | KYG | |
| LanD-D39S w/o | DDKAACA [symbol] GI AAVKARVEKL APEAVPQKLK RALKIAEREQ GEGEFDECLE ALDDAKRALP | 35 |
| signal peptide | KYG | |
| LanD-R47K w/o | DDKAACADGI AAVKA [symbol] VEKL APEAVPQKLK RALKIAEREQ GEGEFDECLE ALDDAKRALP | 36 |
| signal peptide | KYG | |
| LanD-E75Q w/o | DDKAACADGI AAVKARVEKL APEAVPQKLK RALKIAEREQ GEG [symbol] FDECLE ALDDAKRALP | 37 |
| signal peptide | KYG | |
| LanD-E78A w/o | DDKAACADGI AAVKARVEKL APEAVPQKLK RALKIAEREQ GEGEFD [symbol] CLE ALDDAKRALP | 38 |
| signal peptide | KYG | |
| LanD-E78N w/o | DDKAACADGI AAVKARVEKL APEAVPQKLK RALKIAEREQ GEGEFD [symbol] CLE ALDDAKRALP | 39 |
| signal peptide | KYG | |
| LanD-E78Q w/o | DDKAACADGI AAVKARVEKL APEAVPQKLK RALKIAEREQ GEGEFD [symbol] CLE ALDDAKRALP | 40 |
| signal peptide | KYG | |
| LanD-E78D w/o | DDKAACADGI AAVKARVEKL APEAVPQKLK RALKIAEREQ GEGEFD [symbol] CLE ALDDAKRALP | 41 |
| signal peptide | KYG | |
| JanD-E75Q/E78A w/c | DDKAACADGI AAVKARVEKL APEAVPQKLK RALKIAEREQ GEGQFD [symbol] CLE ALDDAKRALP | 42 |
| signal peptide | KYG | |
| SanD-E75Q/E78N w/c | DDKAACADGI AAVKARVEKL APEAVPQKLK RALKIAEREQ GEG [symbol] FD [symbol] CLE ALDDAKRALP | 43 |
| signal peptide | KYG | |
| JanD-D77N/E81A w/c | DDKAACADGI AAVKARVEKL APEAVPQKLK RALKIAEREQ GEG [symbol] FNECL [symbol] ALDDAKRALP | 44 |
| signal peptide | KYG | |
| JanD-D77Q/E81Q w/c | DDKAACADGI AAVKARVEKL APEAVPQKLK RALKIAEREQ GEGEF [symbol] ECL [symbol] ALDDAKRALP | 45 |
| signal peptide | KYG | |
| LanD-D77K/E81@ w/c | DDKAACADGI AAVKARVEKL APEAVPQKLK RALKIAEREQ GEGEF [symbol] ECL [symbol] ALDDAKRALP | 46 |
| signal peptide | KYG | |
| JanD-E75Q/E78T w/c | DDKAACADGI AAVKARVEKL APEAVPQKLK RALKIAEREQ GEG [symbol] ED [symbol] CLE ALDDAKRALP | 47 |
| signal peptide | KYG | |
| LanD- | DDKAACADGI AAVKARVEKL APEAVPQKLK RALKIAEREQ GEG [symbol] FD [symbol] CLE ALSDAKRALP | 48 |
| E75Q/E78A/D84E w/c | KYG | |
| signal peptide | | |
| JanD-E75M/E78A w/c | DDKAACADGI AAVKARVEKL APEAVPQKLK RALKIAEREQ GEG [symbol] FD [symbol] CLE ALDDAKRALP | 49 |
| signal peptide | KYG | |
| M. extorquens AM1 | DDKAACADGI AAVKARVEKL APEAVPQKLK RALKIAEREQ GEGEFDECLE ALDDAKRALP | 50 |
| w/o signal peptide | K | |
| Signal peptide | MMRTRTSLAV PRGFRESALL ALVVLASPAL A | 51 |
| LanD-Mutant with | MMRTRTSLAV PRGFRGSALL ALVVLATPAL ADDKAACAXG IAAVKAXVEK LAPEAVPQKL | 52 |
| Signal Peptide | KRALKIAERE QGEGXFXXCL XALXDAKRAL PKXX | |
| Metal Motif | REXXEXEXDE C | 53 |

Any one of the preceding proteins or proteins comprising any one of the preceding sequences may be affixed or disposed on a substrate.

The following Statements provide various examples of the present example: Statement 1. A metal-binding protein comprising the following sequence:

(SEQ ID NO: 1)
DDKAACAX¹GIAAVKAX²VEKLAPEAVPQKLKRALKI
AEREQGEGX³FX⁴X⁵CLX⁶ALX⁷DAKRALPKX⁸X⁹,

wherein X¹ is D or S; X² is R or K; X³ is E, Q, or M; X⁴ is D, N, Q, or K; X⁵ is E, N, Q, D. A, or T; X⁶ is E, A, or Q; X⁷ is D or E; X⁸ is Y, W, or absent; and X⁹ is G or absent, and or a protein having at least 75% identity to SEQ ID NO:1, wherein the metal-binding protein is optionally disposed or affixed to a substrate, wherein when the metal-binding protein is SEQ ID NO: 50 the metal-binding protein is disposed or affixed to the substrate.
Statement 2. A metal-binding protein according to Statement 1, wherein the protein is disposed or affixed to the substrate.
Statement 3. A metal-binding protein according to Statement 1 or Statement 2, wherein the substrate is a bead (e.g., agarose, silica, polymeric resin, and the like), a membrane, a hydrogel, a protein-based material, a porous framework (e.g., MOF), or the like, or other substrates known in the art.
Statement 4. A metal-binding protein according to any one of the preceding Statements, wherein the protein further comprises a signal sequence.
Statement 5. A metal-binding protein according to Statement 4, wherein the signal sequence is MMRTRTSLAVPRGFRGSALLALVVLATPALA (SEQ ID NO:52).
Statement 6. A metal-binding protein according to any one of the preceding Statements, wherein the metal-binding protein is or comprises any one of the following sequences:

| | SEQ |
|---|---|
| | ID |
| Sequence | O |
| DDKAACAXGI AAVKAXVEKL APEAVPQKLK RALKIAEREQ | 1 |
| GEGXFXXCLX ALXDAKRALP KXX | |
| MMRTRTSLAV PRGFRGSALL ALVVLATPAL ADDKAACADG | 2 |
| IAAVKARVEK LAPEAVPQKL KRALKIAERE QGEGEFDECL | |
| EALDDAKRAL PKYG | |
| MMRTRTSLAV PRGFRGSALL ALVVLATPAL ADDKAACASG | 3 |
| IAAVKARVEK LAPEAVPQKL KRALKIAERE QGEGEFDECL | |
| EALDDAKRAL PKYG | |
| MMRTRTSLAV PRGFRGSALL ALVVLATPAL ADDKAACADG | 4 |
| IAAVKAKVEK LAPEAVPQKL KRALKIAERE QGEGEFDECL | |
| EALDDAKRAL PKYG | |
| MMRTRTSLAV PRGFRGSALL ALVVLATPAL ADDKAACADG | 5 |
| IAAVKARVEK LAPEAVPQKL KRALKIAERE QGEGQFDECL | |
| EALDDAKRAL PKYG | |
| MMRTRTSLAV PRGFRGSALL ALVVLATPAL ADDKAACADG | 6 |
| IAAVKARVEK LAPEAVPQKL KRALKIAERE QGEGEFDACL | |
| EALDDAKRAL PKYG | |
| MMRTRTSLAV PRGFRGSALL ALVVLATPAL ADDKAACADG | 7 |
| IAAVKARVEK LAPEAVPQKL KRALKIAERE QGEGEFDNCL | |
| EALDDAKRAL PKYG | |
| MMRTRTSLAV PRGFRGSALL ALVVLATPAL ADDKAACADG | 8 |
| IAAVKARVEK LAPEAVPQKL KRALKIAERE QGEGEFDQCL | |
| EALDDAKRAL PKYG | |
| MMRTRTSLAV PRGFRGSALL ALVVLATPAL ADDKAACADG | 9 |
| IAAVKARVEK LAPEAVPQKL KRALKIAERE QGEGEFDDCL | |
| EALDDAKRAL PKYG | |
| MMRTRTSLAV PRGFRGSALL ALVVLATPAL ADDKAACADG | 10 |
| IAAVKARVEK LAPEAVPQKL KRALKIAERE QGEGQFDACL | |
| EALDDAKRAL PKYG | |
| MMRTRTSLAV PRGFRGSALL ALVVLATPAL ADDKAACADG | 11 |
| IAAVKARVEK LAPEAVPQKL KRALKIAERE QGEGQFDNCL | |
| EALDDAKRAL PKYG | |
| MMRTRTSLAV PRGFRGSALL ALVVLATPAL ADDKAACADG | 12 |
| IAAVKARVEK LAPEAVPQKL KRALKIAERE QGEGEFNECL | |
| AALDDAKRAL PKYG | |
| MMRTRTSLAV PRGFRGSALL ALVVLATPAL ADDKAACADG | 13 |
| IAAVKARVEK LAPEAVPQKL KRALKIAERE QGEGEFQECL | |
| QALDDAKRAL PKYG | |
| MMRTRTSLAV PRGFRGSALL ALVVLATPAL ADDKAACADG | 14 |
| IAAVKARVEK LAPEAVPQKL KRALKIAERE QGEGEFKECL | |
| QALDDAKRAL PKYG | |
| MMRTRTSLAV PRGFRGSALL ALVVLATPAL ADDKAACADG | 15 |
| IAAVKARVEK LAPEAVPQKL KRALKIAERE QGEGQFDTCL | |
| EALDDAKRAL PKYG | |
| MMRTRTSLAV PRGFRGSALL ALVVLATPAL ADDKAACADG | 16 |
| IAAVKARVEK LAPEAVPQKL KRALKIAERE QGEGQFDACL | |
| EALEDAKRAL PKYG | |
| MMRTRTSLAV PRGFRGSALL ALVVLATPAL ADDKAACADG | 17 |
| IAAVKARVEK LAPEAVPQKL KRALKIAERE QGEGMEDACL | |
| EALDDAKRAL PKYG | |
| DDKAACADGI AAVKARVEKL APEAVPQKLK RALKIAEREQ | 34 |
| GEGEFDECLE ALDDAKRALP KYG | |
| DDKAACASGI AAVKARVEKL APEAVPQKLK RALKIAEREQ | 35 |
| GEGEFDECLE ALDDAKRALP KYG | |
| DDKAACADGI AAVKAKVEKL APEAVPQKLK RALKIAEREQ | 36 |
| GEGEFDECLE ALDDAKRALP KYG | |
| DDKAACADGI AAVKARVEKL APEAVPQKLK RALKIAEREQ | 37 |
| GEGQFDECLE ALDDAKRALP KYG | |
| DDKAACADGI AAVKARVEKL APEAVPQKLK RALKIAEREQ | 38 |
| GEGEFDACLE ALDDAKRALP KYG | |
| DDKAACADGI AAVKARVEKL APEAVPQKLK RALKIAEREQ | 39 |
| GEGEFDNCLE ALDDAKRALP KYG | |
| DDKAACADGI AAVKARVEKL APEAVPQKLK RALKIAEREQ | 40 |
| GEGEFDQCLE ALDDAKRALP KYG | |
| DDKAACADGI AAVKARVEKL APEAVPQKLK RALKIAEREQ | 41 |
| GEGEFDDCLE ALDDAKRALP KYG | |
| DDKAACADGI AAVKARVEKL APEAVPQKLK RALKIAEREQ | 42 |
| GEGQFDACLE ALDDAKRALP KYG | |
| DDKAACADGI AAVKARVEKL APEAVPQKLK RALKIAEREQ | 43 |
| GEGQFDNCLE ALDDAKRALP KYG | |
| DDKAACADGI AAVKARVEKL APEAVPQKLK RALKIAEREQ | 44 |
| GEGEFNECLA ALDDAKRALP KYG | |
| DDKAACADGI AAVKARVEKL APEAVPQKLK RALKIAEREQ | 45 |
| GEGEFQECLQ ALDDAKRALP KYG | |
| DDKAACADGI AAVKARVEKL APEAVPQKLK RALKIAEREQ | 46 |
| GEGEFKECLQ ALDDAKRALP KYG | |
| DDKAACADGI AAVKARVEKL APEAVPQKLK RALKIAEREQ | 47 |
| GEGQFDTCLE ALDDAKRALP KYG | |
| DDKAACADGI AAVKARVEKL APEAVPQKLK RALKIAEREQ | 48 |
| GEGQFDACLE ALEDAKRALP KYG | |
| DDKAACADGI AAVKARVEKL APEAVPQKLK RALKIAEREQ | 49 |
| GEGMEDACLE ALDDAKRALP KYG | |
| DDKAACADGI AAVKARVEKL APEAVPQKLK RALKIAEREQ | 50 |
| GEGEFDECLE ALDDAKRALP K | |
| MMRTRTSLAV PRGFRGSALL ALVVLATPAL ADDKAACAXG | 52 |
| IAAVKAXVEK LAPEAVPQKL KRALKIAERE QGEGXFXXCL | |
| XALXDAKRAL PKXX | |

Statement 7. A metal-binding protein according to any one of the preceding Statements, wherein the metal-binding protein is or comprises:

(SEQ ID NO: 10)
MMRTRTSLAVPRGFRGSALLALVVLATPALADDKAACAD
GIAAVKARVEKLAPEAVPQKLKRALKIAEREQGEGQFDA
CLEALDDAKRALPKYG.

Statement 8. A device (e.g., a filter, membrane, sensor, handheld detector, plate reader, fluorimeter, biosensor, in-line monitor, or the like) comprising a metal-binding protein according to any one of the preceding claims.
Statement 9. A kit comprising the metal-binding protein according to any one of Statements 1 to 8 or materials to prepare a device comprising the metal-binding protein according to any one of Statements 1 to 8.
Statement 10. A method for binding lanthanide ions and/or actinide ions to a protein comprising contacting a metal-binding protein according to any one of Statements 1 to 8 with a sample comprising or suspected of comprising the lanthanide ions and/or actinide ions, wherein (if present) the lanthanide ions and/or actinide ions bind to one or more metal-binding proteins according to any one of Statements 1 to 8.
Statement 11. A method according to Statement 10, wherein the lanthanide ions and/or actinide ions are chosen from La, Ce, Pr, Nd, Pm, Sm, Eu, Gd, Tb, Dy, Ho, Er, Tm, Yb, Lu, Sc, Y, and any combination thereof.
Statement 12. A method according to Statement 10 or Statement 11, wherein the sample is drinking water, wastewater, ground water, ash ponds, aqueous extract from contaminated soil, drainage, leachate, aqueous extract or leachate from a solid waste, or a solid sample.
Statement 13. A method according to any one of Statements 10 to 12, further comprising isolating the one or more metal-binding proteins having one or more lanthanide ions and/or actinide ions bound thereto.
Statement 14. A method according to any one of Statements 10 to 13, wherein a plurality of different the lanthanide ions and/or actinide ions are bound to the metal-binding proteins.
Statement 15. A method according to any one of Statements 10 to 14, wherein each different lanthanide ion and/or actinide ion is separated individually from the metal-binding protein.
Statement 16. A metal-binding protein or peptide comprising one or more metal-binding motifs, wherein at least one of the one or more metal-binding motifs comprises the sequence REX¹X²EX³EX⁴DEC (SEQ ID NO:53), wherein, X¹ is any amino acid; X² is any amino acid; X³ is any amino acid; and X⁴ is F or Y, or a protein having at least 75% identity thereto and the cysteine forms a disulfide bond with a second cysteine elsewhere in the metal-binding protein or peptide. In various examples, the peptide is SEQ ID NO:53 and at least one of X¹, X², or X³ are a cysteine.
Statement 17. A metal-binding protein or peptide according to Statement 16, wherein X² is G, A, K, or R.
Statement 18. A metal-binding protein or peptide according to Statement 16 or Statement 17, wherein X³ is G, A, or K.
Statement 19. A metal-binding protein or peptide according to Statement 16, wherein the metal-binding protein is or comprises any one of the following sequences:

| | SEQ ID |
|---|---|
| Sequence | NO |
| MMRTRTSLAV PRGFRGSALL ALVVLATPAL ADDKAACADG IAAVKARVEK | 2 |
| LAPEAVPQKL KRALKIAERE QGEGEFDECL EALDDAKRAL PKYG | |
| MMRTRTSLAV PRGERGSALL ALVVLATPAL ADDKAACASG IAAVKARVEK | 3 |
| LAPEAVPQKL KRALKIAERE QGEGEFDECL EALDDAKRAL PKYG | |
| MMRTRTSLAV PRGFRGSALL ALVVLATPAL ADDKAACADG IAAVKAKVEK | 4 |
| LAPEAVPQKL KRALKIAERE QGEGEFDECL EALDDAKRAL PKYG | |
| MMRTRTSLAV PRGFRGSALL ALVVLATPAL ADDKAACADG IAAVKARVEK | 5 |
| LAPEAVPQKL KRALKIAERE QGEGQFDECL EALDDAKRAL PKYG | |
| DDKAACADGI AAVKARVEKL APEAVPQKLK RALKIAEREQ GEGEFDECLE | 34 |
| ALDDAKRALP KYG | |
| DDKAACASGI AAVKARVEKL APEAVPQKLK RALKIAEREQ GEGEFDECLE | 35 |
| ALDDAKRALP KYG | |
| DDKAACADGI AAVKAKVEKL APEAVPQKLK RALKIAEREQ GEGEFDECLE | 36 |
| ALDDAKRALP KYG | |
| DDKAACADGI AAVKARVEKL APEAVPQKLK RALKIAEREQ GEGOFDECLE | 37 |
| ALDDAKRALP KYG | |
| DDKAACADGI AAVKARVEKL APEAVPQKLK RALKIAEREQ GEGEFDECLE | 50 |
| ALDDAKRALP K | |

### or any one of SEQ ID NOs: 19 to 33.

Statement 20. A metal-binding protein or peptide according to any one of Statements 16 to 19, wherein the protein is disposed or affixed to the substrate.
Statement 21. A metal-binding protein or peptide according to Statement 20, wherein the substrate is a bead (e.g., agarose, silica, polymeric resin, and the like), a membrane, a hydrogel, a protein-based material, a porous framework (e.g., MOF), or the like, or other substrates known in the art.
Statement 22. A device comprising a metal-binding protein or peptide according to any one of Statements 16 to 19.
Statement 23. A device according to Statement 22, wherein the device is a filter, membrane, sensor, handheld detector, plate reader, fluorimeter, biosensor, or in-line monitor.
Statement 24. A kit comprising the metal-binding protein according to any one of Statements 16 to 19 or materials to prepare a device comprising the metal-binding protein according to any one of Statements 16 to 19.
Statement 25. A method of binding lanthanide ions and/or actinide ions to a protein comprising contacting a metal-binding protein or peptide according to any one of Statements 16 to 19 with a sample comprising or suspected of comprising lanthanide ions and/or actinide ions, wherein the lanthanide ions and/or actinide ions binds to one or more metal-binding proteins or peptides according to any one of Statements 16 to 19.
Statement 26. A method according to Statement 25, wherein the lanthanide ions and/or actinide ions are chosen from La, Ce, Pr, Nd, Pm, Sm, Eu, Gd, Tb, Dy, Ho, Er, Tm, Yb, Lu, Sc, Y, and any combination thereof.
Statement 27. A method according to Statement 25 or Statement 26, wherein the sample is drinking water, wastewater, ground water, ash ponds, aqueous extract from contaminated soil, drainage, leachate, aqueous extract or leachate from a solid waste, or a solid sample.
Statement 28. A method according to any one of Statements 25 to 27, further comprising isolating the one or more metal-binding proteins having one or more lanthanide ions and/or actinide ions bound thereto.
Statement 29. A method according to any one of Statements 25 to 28, wherein a plurality of different lanthanide ions and/or actinide ions are bound to the metal-binding protein or peptide.
Statement 30. The method according to claim 33, wherein each different lanthanide ion and/or actinide ion is separated individually from the metal-binding protein or peptide.

The following examples are presented to illustrate the present disclosure. They are not intended to be limiting in any matter.

### EXAMPLE 1

This example provides a description of peptides and proteins of the present disclosure.

Provided are structurally and biochemically characterize LanD, leading to the following conclusions:
M. extorquens LanD dimerizes in a REE-sensitive manner, with light lanthanides inducing the highest affinity dimers.
Unlike Hans-LanM, however, the metal-binding site in LanD is comprised symmetrically of residues from each monomer, and represents the exclusive bridge between the two monomers. X-ray crystal structures show that E70, E73, and E75 from each monomer contribute ligands. For La(III) but not Eu(III), there is an additional solvent molecule.
The single site removes the potential for heterometallic complexes forming, as can occur in LanM. The metal affinity also is weaker than in LanMs.

### Expression and Purification of LanD and its D39S and R47K Variants.

M. extorquens LanD was expressed from pET24a-LanD (pET24a-p1781), which was described previously. This construct contains a C-terminal Tyr-Gly addition (to facilitate protein quantification as the native protein contains no Tyr or Trp residues). Electrocompetent E, coli BL21 (DE3) cells were transformed with pET24a-LanD and plated on LB-agar plates containing 50 μg/mL kanamycin (Km) and incubated at 37° C. A single colony was used to inoculate 100 mL of LB (50 μg/mL Km in all growth media), which was grown for ˜16 h at 37° C. with shaking at 200 rpm. This culture was used to inoculate three 2 L cultures (in 6 L flasks). The cultures were grown at 37° C. with shaking at 170 rpm. At OD₆₀₀ₙₘ˜0.6, isopropyl-β-D-thiogalactopyranoside (IPTG. Oakwood Chemical) was added to a final concentration of 2 μM and the cultures were further incubated at 20° C. for ˜16 h. (The lower concentration of IPTG is an improvement of the previously published protocol, and it leads to substantially higher yields of purified protein.) The cells were pelleted by centrifugation for 7 min at 7000×g at 4° C., yielding ˜5 g cell paste per L culture. The periplasmic extract was prepared using the cold osmotic shock method, as described. The periplasmic extract in 5 mM MgSO₄ was buffered by addition of 0.05 volumes of 1 M Tris, pH 7.4 and filtered through a 0.2 μm polyethersulfone (PES) membrane. This solution was applied to a 2.5×4.5 cm (20 mL) Q-Sepharose Fast Flow column that had been pre-equilibrated in 50 mM Tris, 1 mM EDTA, pH 7.0 (Buffer A). The column was washed with 1 CV Buffer A, and the protein was eluted with 5 CV Buffer A containing 50 mM NaCl and 5 CV Buffer A containing 100 mM NaCl. LanD-containing fractions were determined by SDS-PAGE gel analysis. The column wash and elution fractions were concentrated to 5 mL using an Amicon Ultra-15 3-kDa MWCO centrifugal filter. LanD was separated from higher molecular weight proteins and the buffer was exchanged into 20 mM MES, 100 mM KCl. 5 mM acetate, pH 6.0 (Buffer B), by size-exclusion chromatography on a HiLoad 16/600 Superdex 75 pg column. The protein sample was loaded onto the column using a 5-mL capillary loop and eluted with 1.2 CV Buffer B. Fractions (1 mL) were collected in peak fractionation mode with a 1 mAU threshold at 280 nm. LanD eluted at 73-80 mL. LanD-containing fractions were dialyzed against 20 mM Tris, 100 mM KCl, pH 7.0 containing Chelex-100 as described for LanM. The extinction coefficient of LanD (with the C-terminal YG addition) was determined to be ε₂₇₅ₙₘ=1430 M⁻¹ cm⁻¹ by correlation of UV-visible absorption spectra and Direct Detect measurements (Automated Biological calorimetry Facility). The purification yielded 14 mg LanD per L culture.

The gene encoding M. extorquens LanD, codon optimized for expression in E. coli with the D39S or R47K mutation and a C-terminal YG addition, was obtained from Twist Bioscience inserted into the NdeI/XhoI sites of pET-29b(+). Expression and purification of these variants was conducted using the same procedure as above, yielding 10 mg per L culture (D39S) and 11 mg per L culture (R47K). Amino acid sequences of these proteins are presented in Table 1.

TABLE 1
Comparison of the amino sequences of LanD and some of the
variants used in this disclosure. The signal peptide is
underlined, the C-terminal YG addition is in bold,
and point mutations are bolded, enlarged, and in italics.

| | | SEQ |
|---|---|---|
| | | ID |
| | Amino Acid Sequence | NO: |
| WT | MMRTRTSLAV PRGFRGSALL ALVVLATPAL ADDKAACADG | 2 |
| | IAAVKARVEK LAPEAVPQKL KRALKIAERE QGEGEFDECL | |
| | EALDDAKRAL PKYG | |
| D39S | MMRTRTSLAV PRGFRGSALL ALVVLATPAL ADDKAACA [symbol] G | 3 |
| | IAAVKARVEK LAPEAVPQKL KRALKIAERE QGEGEFDECL | |
| | EALDDAKRAL PKYG | |
| | MMRTRTSLAV PRGFRGSALL ALVVLATPAL ADDKAACADG | 4 |
| R47K | IAAVKA [symbol] VEK LAPEAVPQKL KRALKIAERE QGEGEFDECL | |
| | EALDDAKRAL PKYG | |
| E75Q | MMRTRTSLAV PRGFRGSALL ALVVLATPAL ADDKAACADG | 5 |
| | IAAVKARVEK LAPEAVPQKL KRALKIAERE QGEG [symbol] FDECL | |
| | EALDDAKRAL PKYG | |

### La(III)-LanD X-Ray Crystallography Data Collection and Structure Determination.

Purified M. extorquens LanD was exchanged into 30 mM MOPS and 50 mM KCl, pH 7, prior to crystallization. The protein was loaded with 0.5 equivalents of La(III) (LaCl₃) per monomer. Crystals were obtained by using the sitting drop vapor diffusion method, in which 1 μL of protein solution (19.3 mg/mL) was mixed with 1 μL 100 mM MES, pH 6.5, and 50% (w/v) PEG 200 in a 24-well plate from Hampton Research (cat, no. HR1-002) at room temperature. Cubic-shaped crystals appeared in one month. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Life Sciences Collaborative Access Team (LS-CAT) ID-G beamline and processed with the HKL2000 package. La(III)-bound LanD crystallized in the I2₁3 space group (β=90.00°) with 1 monomer. The structure was solved using the single-wavelength anomalous diffraction (SAD) method. Using a dataset collected at 12.7 keV (0.9792 Å), HySS identified one La(III) site that was subsequently used to obtain phases in phenix.autosol. The initial figure of merit (FOM) and Bayesian CC were 0.442 and 0.52, respectively. An initial model was generated with phenix.autobuild with subsequent rounds of manual modification and refinement in Coot and phenix.refine. In the final stages of model refinement, anisotropic displacement parameters (ADP) were refined for all La(III) sites. The final model afforded an FOM of 0.81 and R_{work}/R_{free} of 0.205/0.236. The final model consists of residues 32-91 in each chain, one La(III) ion, and 50 water molecules. Of the residues modeled. 100% are in allowed or preferred regions as indicated by Ramachandran statistical analysis. Model validation was performed with the Molprobity server. Figures were prepared using the PyMOL molecular graphics software package (Schrödinger. LLC).

### Eu(III)-LanD X-Ray Crystallography Data Collection and Structure Determination.

Purified M. extorquens LanD was exchanged into 30 mM MOPS and 50 mM KCl, pH 7, prior to crystallization. The protein was loaded with 0.5 equivalents of Eu(III) (EuCl₃) per monomer. Crystals were obtained by using the sitting drop vapor diffusion method, in which 1 μL of protein solution (19.3 mg/mL) was mixed with 1 μL 100 mM MES. pH 6.5, and 25% (w/v) PEG 200 in a 24-well plate from Hampton Research (cat, no. HR1-002) at room temperature. Cubic-shaped crystals appeared in one month. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Brookhaven National laboratory (BNL) 17-ID-1 AMX beamline and processed with the HKL2000 package. Eu(III)-bound LanD crystallized in the I2₁3 space group (β=90.00°) with 1 monomer. The structure was solved using the single-wavelength anomalous diffraction (SAD) method. Using a dataset collected at 13.5 keV (0.9201 Å). HySS identified one Eu(III) site that was subsequently used to obtain phases in phenix autosol. The initial figure of merit (FOM) and Bayesian CC were 0.638 and 0.58, respectively. An initial model was generated with phenix.autobuild with subsequent rounds of manual modification and refinement in Coot and phenix.refine. In the final stages of model refinement, anisotropic displacement parameters (ADP) were refined for all Eu(III) sites. The final model afforded an FOM of 0.82 and R_{work}/R_{free} of 0.203/0.229. The final model consists of residues 32-91, one Eu(III) ion, and 28 water molecules. Of the residues modeled. 100% are in allowed or preferred regions as indicated by Ramachandran statistical analysis. Model validation was performed with the Molprobity server. Figures were prepared using the PyMOL molecular graphics software package (Schrödinger. LLC).

### Apo-LanD X-Ray Crystallography Data Collection and Structure Determination.

Purified M. extorquens LanD was exchanged into 20 mM MOPS and 20 mM KCl, pH 7, prior to crystallization. Crystals were obtained by using the sitting drop vapor diffusion method, in which 1 μL of protein solution (19.6 mg/mL) was mixed with 1 μL 0.2 M potassium fluoride, and 20% (w/v) PEG 3350 at room temperature. Thin plate-shaped crystals appeared in three days. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Life Sciences Collaborative Access Team (LS-CAT) ID-G beamline and processed with the HKL2000 package. Apo-LanD crystallized in the C₁2₁ space group with two monomers. The structure was solved using the molecular replacement in PHENIX. The search model was from La(III)-bound LanD without metal and water molecules. The final model afforded an FOM of 0.82 and R_{work}/R_{free} of 0.206/0.252. The final model consists of residues 32-92, and 194 water molecules. Of the residues modeled. 100% are in allowed or preferred regions as indicated by Ramachandran statistical analysis. Model validation was performed with the Molprobity server. Figures were prepared using the PyMOL molecular graphics software package (Schrödinger. LLC).

### Isothermal Titration Calorimetry.

The dissociation constants for the dimers of apo and Nd-, Sm-, and Eu-bound LanD were determined by dilutive additions of a concentrated protein stock, followed using isothermal titration calorimetry on a TA Instruments Low-volume Auto Affinity isothermal titration calorimeter. The syringe contained 800 M protein (apo) or 300 μM (protein with 0.5 equivalents of Ln(III) bound), and the cell contained 185 μL of a matched buffer (20 mM Tris, 100 mM KCl, pH 7.0). Titrations were carried out at 25° C. Titrations consisted of a first 0.2-μL injection followed by 20×2-μl injections with stirring at 125 rpm and 180 s equilibration time between injections. The data were fitted using NanoAnalyze using the Dimer Dissociation model, yielding the dimer dissociation constant (K_{dimer}), enthalpy of dissociation (ΔH) and entropy of dissociation (ΔS).

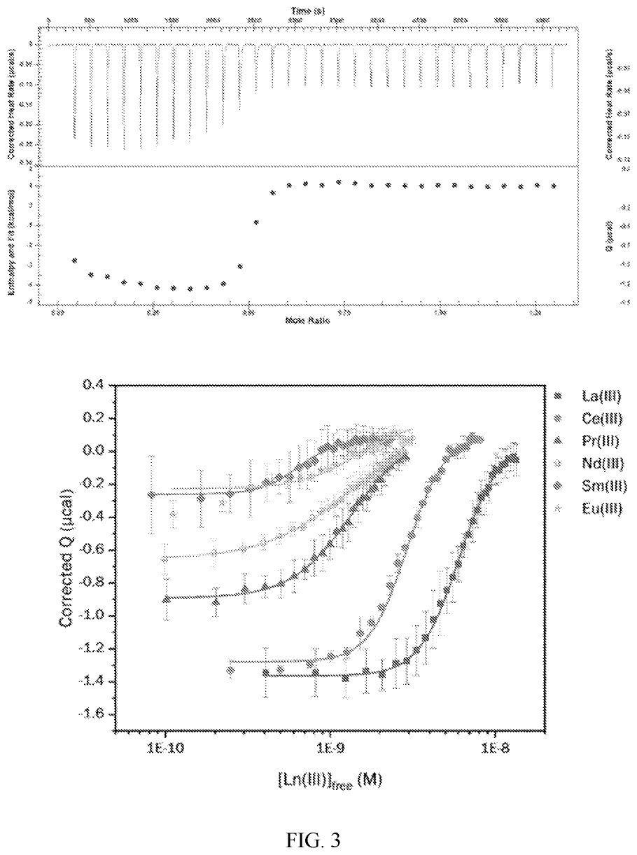

Binding of lanthanides to LanD was characterized using a TA Instruments Low-volume Auto Affinity isothermal titration calorimeter. The ITC cell contained 60 μM LanD in Chelex-treated 20 mM Tris, 100 KCl, pH 7.0 with 500 μM citrate. The citrate was present because prior experiments with La(III) had indicated a stoichiometry of ˜0.5 equivalents but Kas close to the limit of the ITC method (low nanomolar range): in addition, there was some evidence of non-specific metal binding past the endpoint of the first binding event (see FIG. 3, top). Titrations were carried out at 25° C. The titrant syringe contained 400 μM Ln(III) (Ln=La, Ce, Pr, Nd, Sm, Eu) with 500 μM citrate prepared in the same buffer. Titrations consisted of 30×2.0 μL injections. The equilibration times were 240 s between injections, and the sample cell was stirred at 125 rpm. The heats of dilution were determined by titrating the same metal solutions into matched buffer without protein. The corrected heats were determined by subtracting the heats of dilution from the protein data. Using tabulated stability constants for Ln(III) ions complexed to citrate and stability constants of aqueous hydroxo complexes in conjunction with the chemical speciation software Hyperquad simulation and speciation (HySS) we estimated free Ln(III) concentrations at each titration point during the ITC experiments. Note that the (significant) contribution of protein binding to the free Ln(III) concentrations was not considered in this analysis. Consequently, calculated free metal concentrations are likely inaccurate due to complexities in citrate speciation and contributions from protein binding. Therefore, these results were considered qualitatively representative of relative affinities of Ln^{III} complexes with LanD, but not quantitatively representative of dissociation constants. Indeed. FIG. 6B shows a similar trend qualitatively but not quantitatively.

Fluorescence measurements, a) General methods. Time-resolved fluorescence data were collected with a Fluorolog-QM fluorometer in configuration 75-21-C(Horiba Scientific) equipped with a double monochromator on the excitation arm and single monochromator on the emission arm. A pulsed xenon lamp was used for lifetime measurements. All fluorescence data were collected in quartz cuvettes and the emission was collected at 90° relative to the excitation. Lifetimes were fitted using FelixFL software (Horiba Scientific).

Fluorescence measurements, b) Lifetime measurements in D₂O—H₂O mixtures. These measurements were carried out using established methods. Solutions of 20 μM or 350 μM LanD with 0.5 equivalents Eu(III) were prepared in 100% H₂O matrix (Buffer: 20 mM Tris, 100 mM KCl, pH 7.0). This initial 100% H₂O Eu(III)-LanD₂ mixture was split in half (2×1.5 mL aliquots) and dehydrated by lyophilization. The residual solid was rehydrated in an equivalent amount of H₂O or 99.9% D20, lyophilized again, and resuspended a second time in an equivalent amount of H₂O or 99.9% D20. These two Eu(III)-LanD₂ solutions (100% H₂O or ˜99.9% D20) were mixed in varying ratios to produce D20 contents of 0%, 25%, 50%, 75%, and 99.9%. For each mixture, the lifetime was measured (λₑₓ=394 nm, λₑₘ=615 nm) with 2000 shots over a time span of 2500 μs.

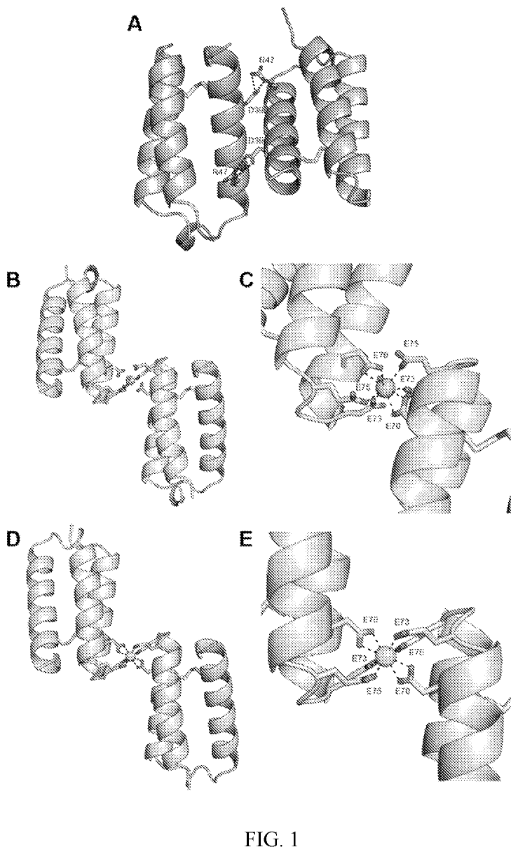

Structural characterization of LanD. The X-ray crystallographic structure of apo-LanD was solved to 1.65 Å (FIG. 1A). The protein adopts a three-helix bundle with helices 1 and 3 bridged by a disulfide bond. The structure of the apoprotein shows a dimer, with an interface between the first helix of each monomeric unit, with only two primary interactions. Both interactions are formed between the Asp39 residue in one monomer and the Arg47 residue in the other. Additionally, an X-ray crystallographic structure of La(III)-bound LanD was solved to 1.91 Å, depicting a dimer interface templated exclusively by the metal ion, on the opposite end of the monomeric unit from the apo interface, with ligands from the loop between helices 2 and 3 (FIG. 1B). The La(III) ion is coordinated by two monodentate Glu residues (E73 and E75) and one bidentate Glu residue (E70) from each monomer. One partially occupied solvent molecule is present as well, overall forming a 9-coordinate complex, typical for larger lanthanides (FIG. 1C). Finally, the X-ray crystallographic structure of Eu(III)-bound LanD was solved to 2.09 Å (FIG. 1D). This structure shows a similar metal-centered dimer interface as the La(III)-bound structure, with some slight differences. Primarily, there is an absence of a coordinated solvent molecule, creating an 8-coordinate complex, typical for smaller lanthanides (FIG. 1E). These observations of the dimerization of apoprotein and formation of a dimer-bridged metal binding site account for the preliminary observations of 1) elution of the protein as a dimer from the SEC at high concentration and 2) the metal-binding stoichiometry of ˜0.5 by our group, at high protein concentrations.

### Apo- and Lanthanide-Dependent Dimerization.

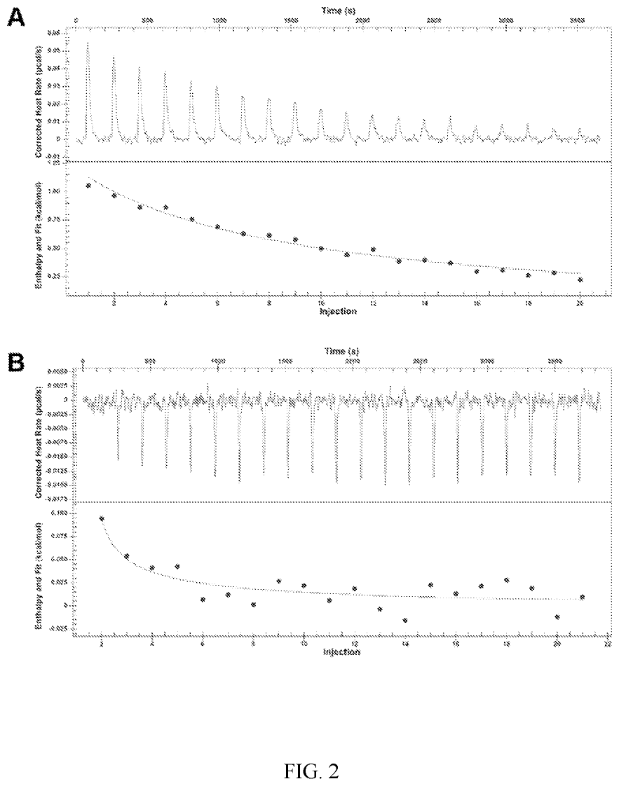

The X-ray crystal structures motivated determination of the dimerization equilibrium constants (K_{dimer}) for the various forms of LanD, using isothermal titration calorimetry (ITC). We previously used this approach to measure metal-dependent dimerization in Hans-LanM. For the apo dimer (FIG. 2A), dimer dissociation is endothermic, with fitting yielding a K_{dimer} value of 420 μM (Table 2). In order to test the relevance of the crystallographic dimer involving hydrogen bonds between D39 and R47, we made mutations to disrupt these interactions (D39S and R47K). K_{dimer} values for the D39S and R47K variants were measured to be 650 μM and 1150 μM, respectively. These weaker values support the crystallographic model. Fitting parameters for these experiments are presented in Table 2.

Similarly, holo-K_{dimer} values were measured for WT LanD bound to Nd(III) (16 μM), Sm(III) (230 μM), or Eu(III) (690 μM) (Table 2, FIG. 2B). Fitting parameters are presented in Table 2. The large uncertainty values in these titrations (˜50%) are likely a result of the low heat changes associated with these reactions: this is not surprising given that the holo dimer interface consists exclusively of metal-ligand bonds. Still, the results suggest a sharp trend in weakening of the dimer, with large differences even between adjacent lanthanides, which would present a means for efficient lanthanide separations by harnessing the different sizes or stabilities of the monomer/dimer complexes. Note that these titrations were carried out in Tris buffer (unlike in Example 2). The background heats associated with the control titration of metal into Tris buffer, which were subtracted from the titration data for titration of metal into protein, are very large and contribute significant uncertainties to these numbers, which motivated similar experiments carried out in MOPS buffer described below.

TABLE 2
Thermodynamic parameters for dissociation of apo- and holo-LanD
and apo-D39S and R47K, obtained by fitting ITC thermograms to
the dimer dissociation model. Uncertainties represent standard
deviations for the mean from three independent experiments.

| | K_{dimer} (μM) | ΔH (kcal/mol) | ΔG (kcal/mol) | ΔS (cal/mol/K) |
|---|---|---|---|---|
| Apo | 420(40) | 4.1(0.1) | 4.6(0.1) | −1.8(0.3) |
| Nd(III) | 16(9) | 1.8(2.0) | 6.5(0.2) | −16(6) |
| Sm(III) | 230(140) | 1.0(0.1) | 5.0(0.4) | −14(2) |
| Eu(III) | 690(430) | 1.6(0.4) | 4.4(0.4) | −9(3) |
| D39S | 600(100) | 3.6(0.2) | 4.4(0.1) | −2.8(0.6) |
| R47K | 1190(170) | 5.6(0.5) | 4.0(0.1) | 5.3(2) |

This sharp decline in dimer affinity, in conjunction with the crystallographic analysis, may account for the previously observed weaker metal binding to the right of roughly Dy(III). Having already lost the solvent molecule forming the 9^{th} coordination site at some point between La(III) and Eu(III), as the metal ions continue to get smaller, they will accommodate the 8 protein ligands increasingly less well. Because the ligation is symmetrical, decreasing coordination number further would mean going to 6-coordination. This, in addition to potential steric clashes as the ionic radius of the metal ion decreases further, would strongly disfavor dimerization at a certain point. The crystallization of Ho(III)-LanD₂ was to test this hypothesis, although it is possible that at the high concentrations of the crystallization condition a holodimer-like conformation may still be favored over an apo-like structure.

### Eu(III) Lifetimes to Monitor Dimerization.

Because of the large uncertainties in the ITC data for the holodimers, Eu(III) luminescence was used to estimate K_{dimer} for Eu(III)-LanD. Empirical equations were previously developed that allow for the estimation of the number of water molecules (q) in the first coordination sphere of a metal ion based on its fluorescence lifetime. Based on these equations, at lower protein concentrations (20 μM), q=4.1, while at 350 μM, q decreases to 2.6. Assuming that the holodimer has no coordinated solvent based on the X-ray structure of Eu(III)-LanD, and that the Eu(III) is fully protein bound in the q=4 condition-then at 350 μM LanD, ˜65% of the protein units are monomeric in solution. Using the equation 2[M]²+K_{dimer}{[M]-[P]}=0, where [M] is the monomer concentration and [P] is the total protein concentration, K_{dimer} is ˜850 μM, with is consistent with the ITC results.

### ITC in Presence of a Competitive Chelator.

Initially in an attempt to obtain insight into metal binding thermodynamics, titrations of LanD with La, Ce, Pr, Nd, Sm, and Eu were carried out in the presence of citrate as a competitive chelator. The results are shown in FIG. 3 (bottom). For earlier Lns such as La(III) and Ce(III), the change in heat (denoted as ΔQ in the figure) over the course of a binding event is relatively large (˜1.4 μcal) compared to smaller Lns like Sm(III) and Eu(III) with ΔQ values of ˜0.2 μcal, while intermediate sized Lns (Pr(III) and Nd(III)) have intermediate ΔQ values. As mentioned in the Methods, the (significant) contribution of protein binding to the free Ln(III) concentrations was not considered in this analysis. This fact, in addition to the fact that the speciation of lanthanide-citrate complexes in solution is complicated, means there are likely large errors in estimation of the free lanthanide ion concentrations (x-axis of the figure). Therefore, we consider these results qualitatively representative of relative affinities of Ln^{III} complexes with LanD, but not quantitatively representative of dissociation constants. FIG. 6B shows a similar trend qualitatively but not quantitatively.

### Size Exclusion Filtration for Separation Application.

Disruption of the apo-dimer while maintaining the holo-dimerization could provide a means for lanthanide separations based on size exclusion. Apoprotein (WT or R47K, 400 μM) was incubated with 0.5 equivalents of La(III) for 10 min on a nutator rocker. The solution was applied to a 5-kDa MWCO or a 10-kDa MWCO centrifugal filtration device and centrifuged for 20 min (5 min for 10-kDa MWCO filters) at 12000×g. Retentate and flowthrough were analyzed by UV-visible spectroscopy to determine the location of protein upon filtration. When utilizing the 5-kDa MWCO filters, all of the protein was retained in the retentate. When utilizing the 10-kDa MWCO filters, all of the protein went through the filter, ending up in the flowthrough. While preliminary experiments have been unsuccessful, perhaps because the dimers may be too transient, this lanthanide-dependent dimerization has the potential of being exploited for separation applications, such as using higher protein concentrations, higher pH to strength interactions, or by increasing the size of the proteins. It is also possible that the separation could be based on affinity of the metal-protein complexes: e.g., the filtration experiment could be run at a pH at which metal M1 binds to the protein (and is stabilized by dimerization) but M2 induces the monomer, is therefore the M2-protein complex is less stable, and dissociates.

### EXAMPLE 2

This example provides a description of proteins and peptides of the present disclosure.

Elucidating details of biology's selective uptake and trafficking of rare earth elements, particularly the lanthanides, has the potential to inspire sustainable biomolecular separations of these essential metals for myriad modern technologies. Here we Methylobacterium (Methylorubrum) extorquens LanD, a periplasmic protein from a bacterial gene cluster, was biochemically and structurally characterized for lanthanide uptake. This protein provides only four ligands at its surface-exposed lanthanide-binding site, allowing for metal-centered protein dimerization that favors the largest lanthanide. La^{III}. However, the monomer prefers Nd^{III} and Sm^{III}, which are disfavored lanthanides for cellular utilization. Structure-guided mutagenesis of a metal ligand and an outer-sphere residue weakens metal binding to the LanD monomer and enhances dimerization for Pr^{III} and Nd^{III} by 100-fold. Selective dimerization enriches high-value Pr^{III} and Nd^{III} relative to low-value La^{III} and Ce^{III} in an all-aqueous process, achieving higher separation factors than lanmodulins, and comparable or better separation factors than common industrial extractants. Finally, we show that LanD interacts with lanmodulin (LanM), a previously characterized periplasmic protein that shares LanD's preference for Nd^{III} and Sm^{III}. These results suggest that LanD's unusual metal-binding site transfers less-desirable lanthanides to LanM to siphon them away from the pathway for cytosolic import. The properties of LanD show how relatively weak chelators can achieve high selectivity, and they form the basis for the design of protein dimers for separation of adjacent lanthanide pairs and other metal ions.

Herein, structural and biochemical studies of LanD reveal an unusual surface binding site with a metal coordination sphere that is only half-saturated by the protein. This allows LanD to form light lanthanide-selective dimers centered on a single metal ion. Two structure-guided substitutions invert dimerization selectivity and achieve separation factors of light lanthanides comparable to industrial extractants. Biochemical studies indicate that, physiologically, LanD's metal site is designed to disfavor self-dimerization while facilitating transfer of Ln^{III} ions to LanM. Therefore, characterization of LanD advances both biomolecular separations and understanding of lanthanide trafficking within cells.

### LanD Forms a Metal-Centered Dimer.

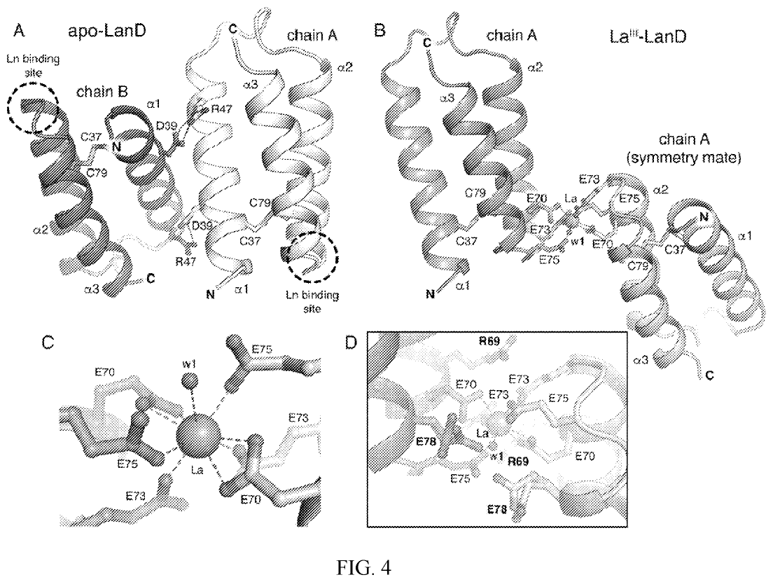

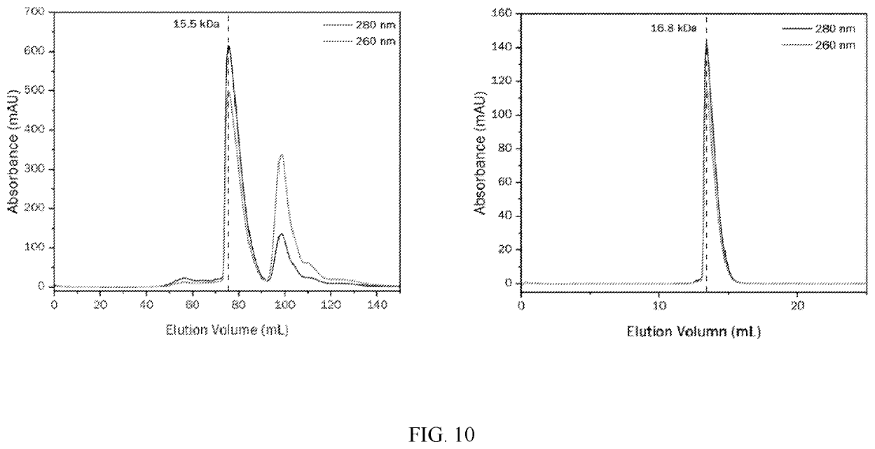

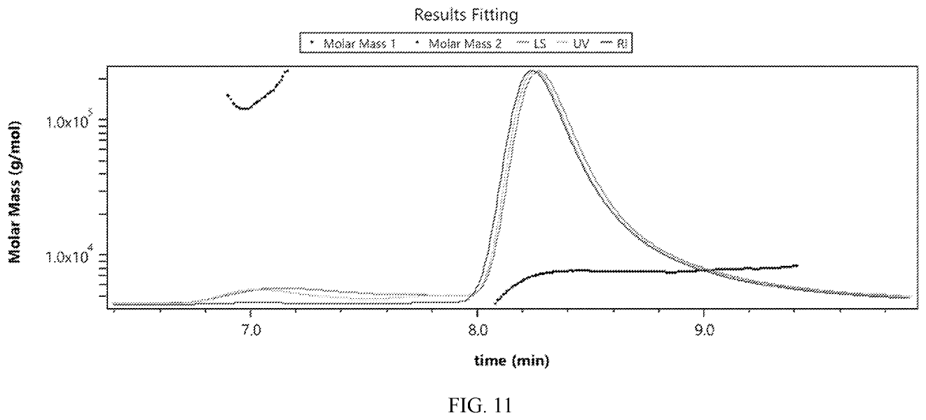

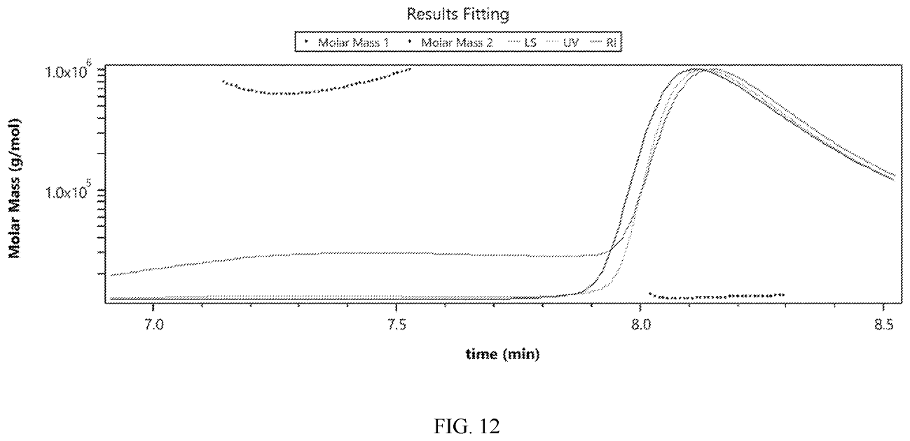

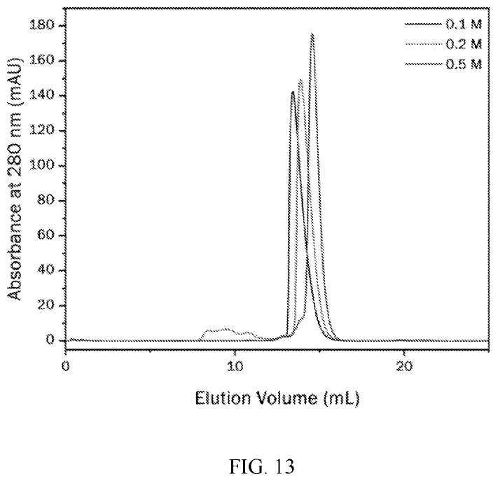

The X-ray crystal structure of LanD (UniprotKB C5B159) in the apo state (FIG. 4A, Table 4) reveals a compact three-helix bundle fold, stabilized by a disulfide linkage between helices 1 and 3 (α1, α3). The asymmetric unit contains two copies of LanD with a dimer interface, involving α1 in each monomer, burying ˜270 Å² of surface area (FIG. 4A). The apo dimer appears to be stabilized primarily via polar contacts, the most significant of which is a pair of symmetric inter-monomer salt-bridge interactions, involving Asp39 in one monomer and Arg47 in the other. Indeed, the behavior of the apoprotein in size-exclusion chromatography experiments (FIGS. 10-12) suggests a dimer at high protein concentration. In solution, the dimer is disrupted by high ionic strength, supporting the relevance of the crystallographically observed Asp39-Arg47 salt bridges (FIG. 13).

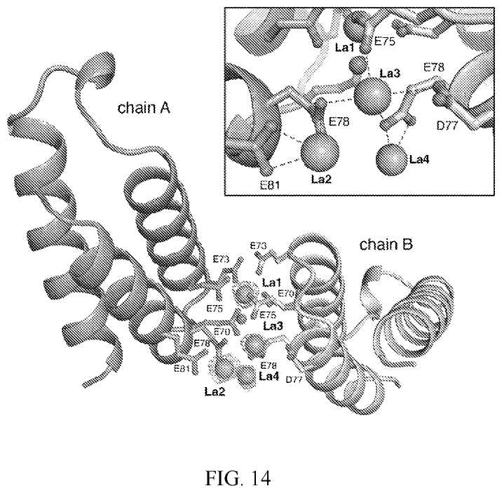

To identify the putative Ln^{III}-binding motif in LanD, the protein was co-crystallized with La^{III}, the lightest Ln^{III} ion. This structure also revealed a dimer, but with an interface distinct from that of the apoprotein (FIG. 4B). Strong anomalous difference electron density map peaks for metal ions near a cluster of carboxylate side chains at the C-terminal end of the central α-helix (α2), proximal to the disulfide linkage (FIG. 14), were observed. Unexpectedly, initial structures solved with 1:1 ratios of La^{III}:LanD revealed both a primary metal binding site and several auxiliary adventitious binding sites (FIG. 14). Because the location of these metal-binding sites appeared to bridge a dimeric quaternary form, the metal:protein ratio was decreased to 0.5 in subsequent crystallization trials. These efforts yielded structures containing only a single metal ion bound at full occupancy at the interface between two LanD monomers (FIG. 4B).

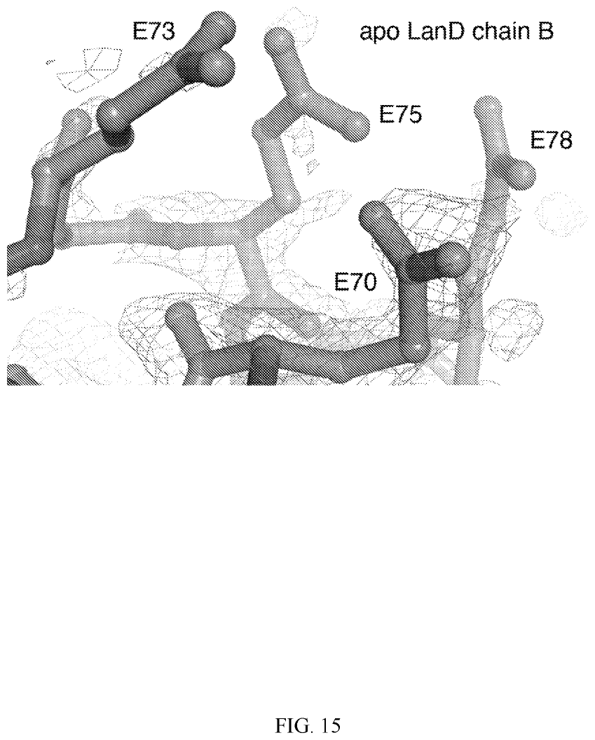

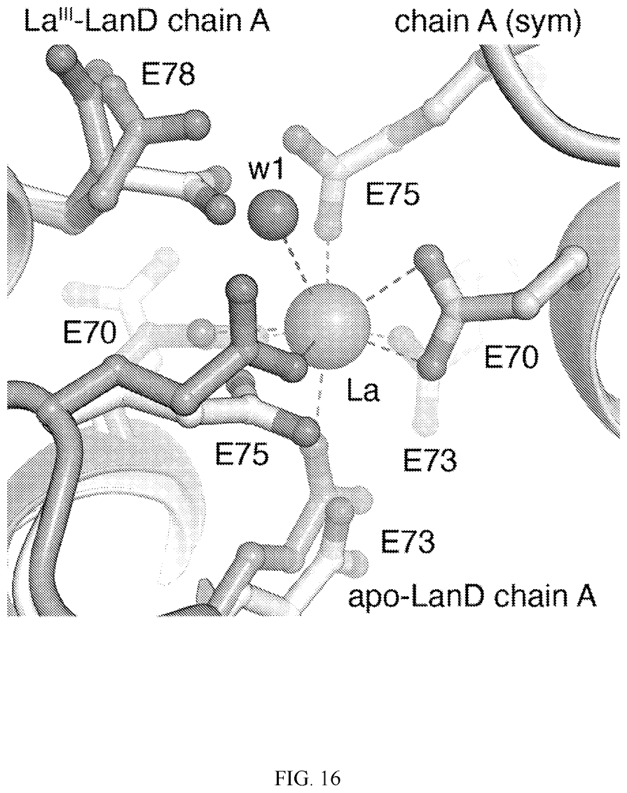

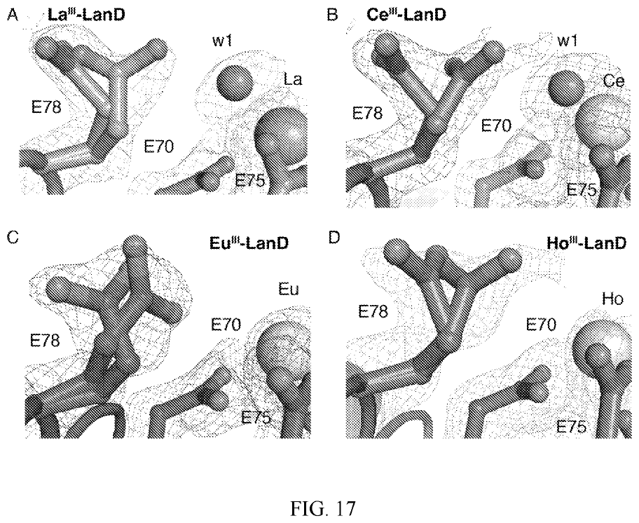

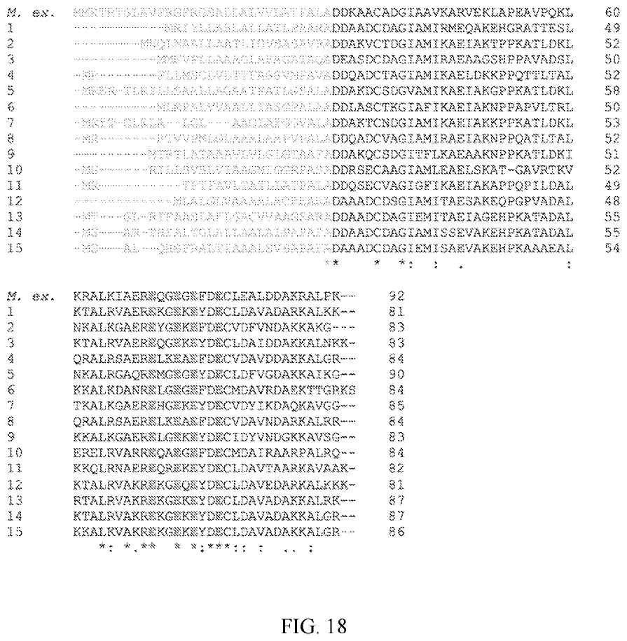

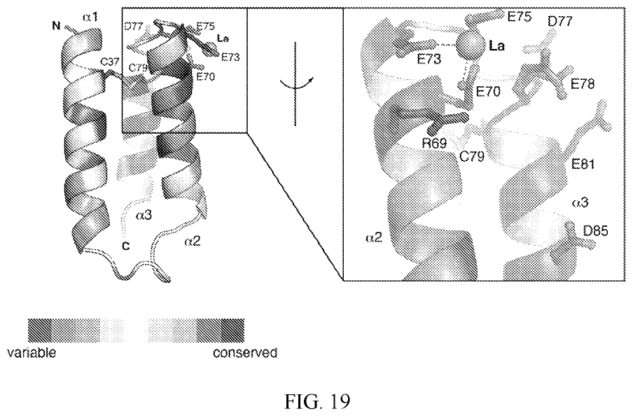

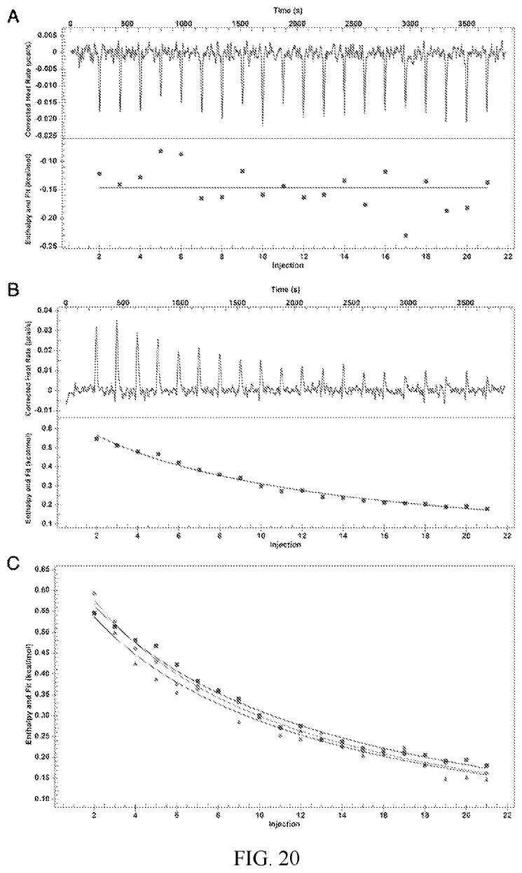

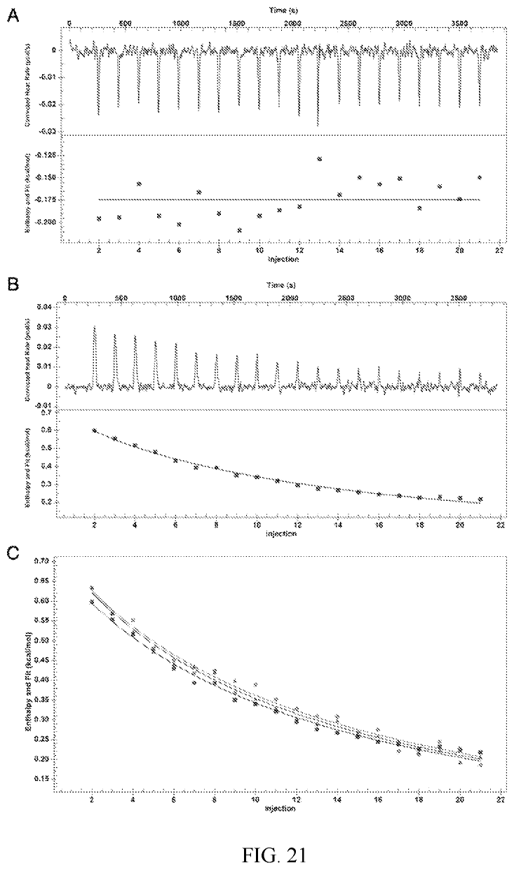

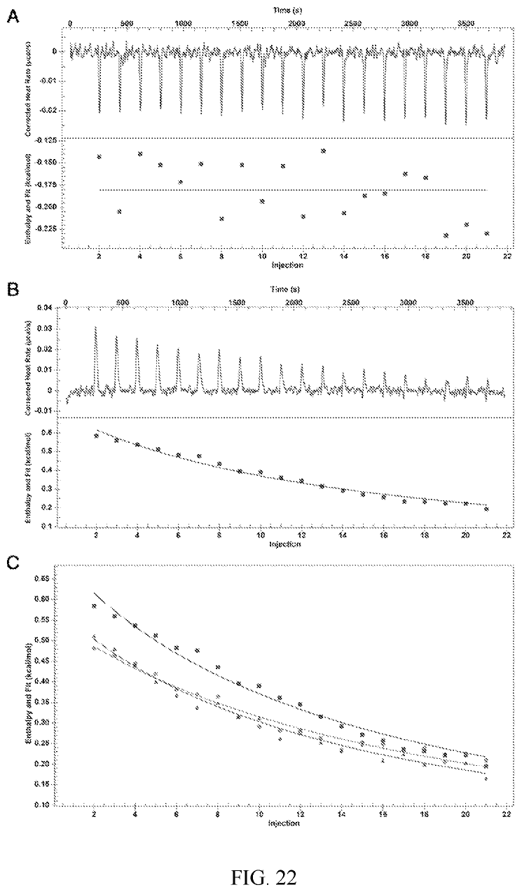

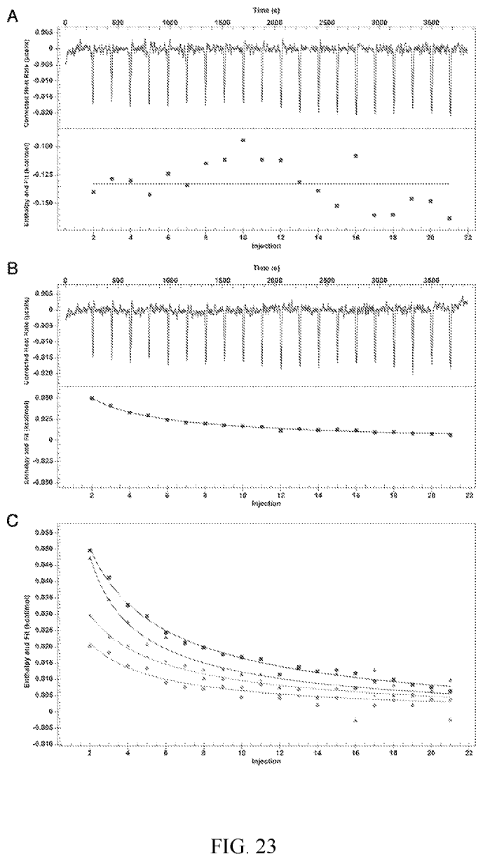

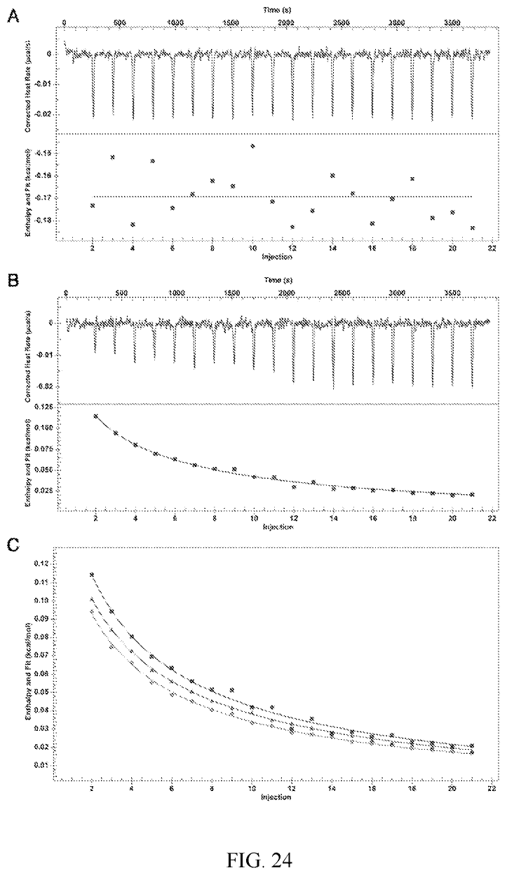

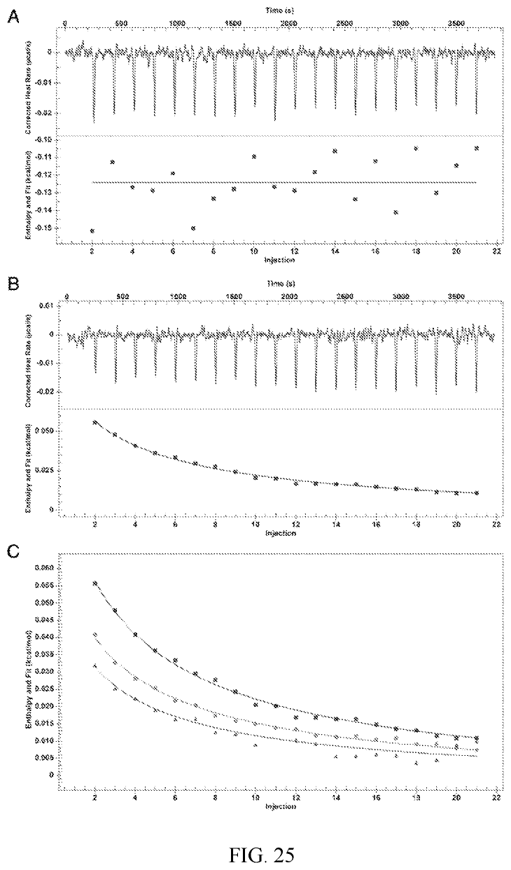

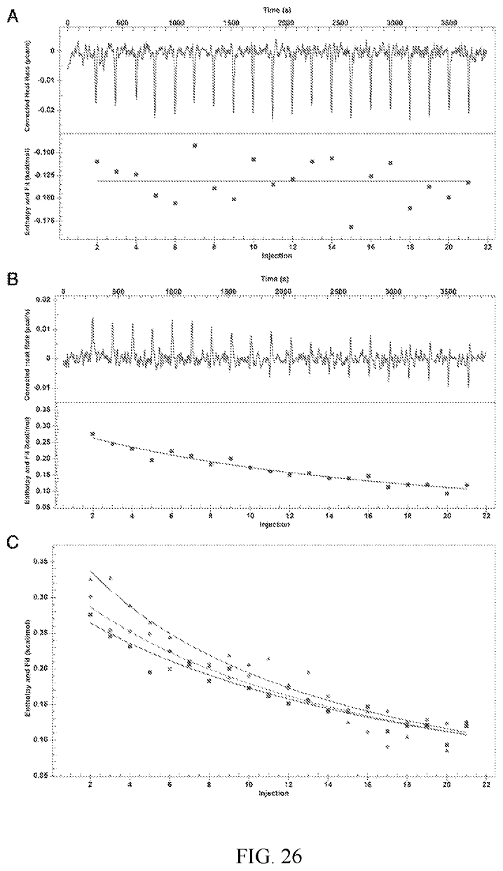

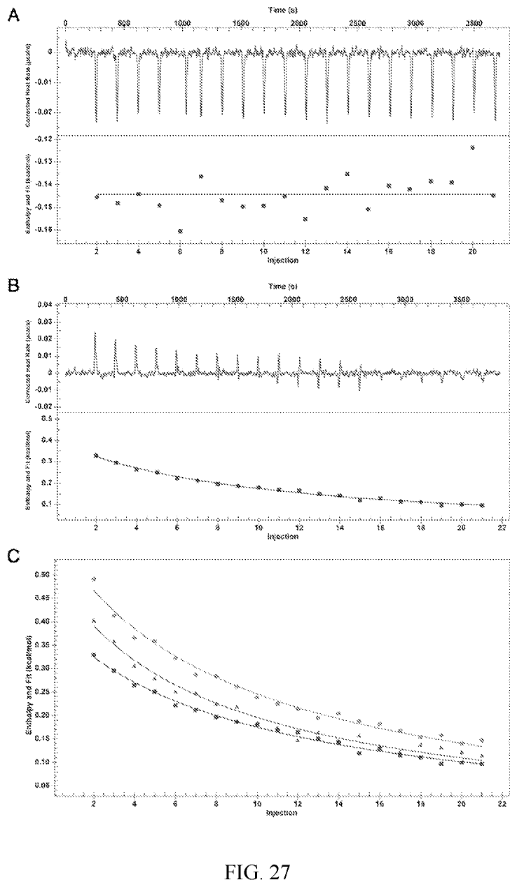

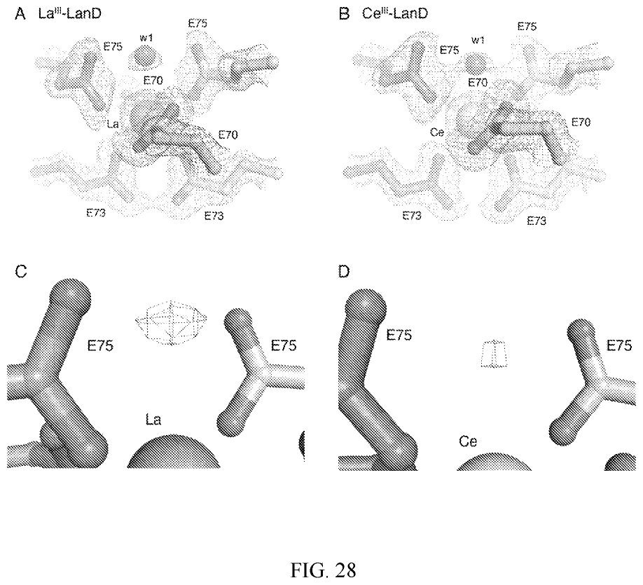

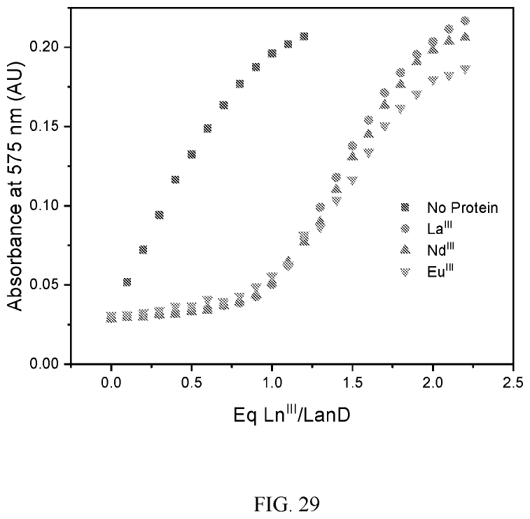

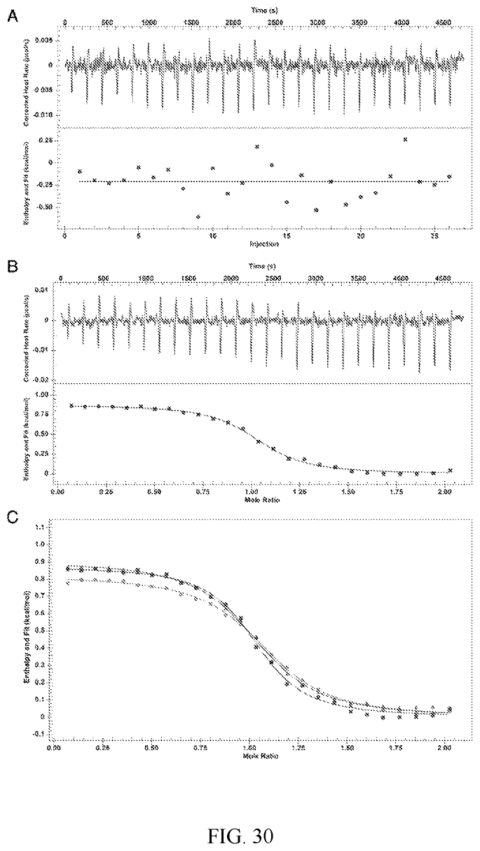

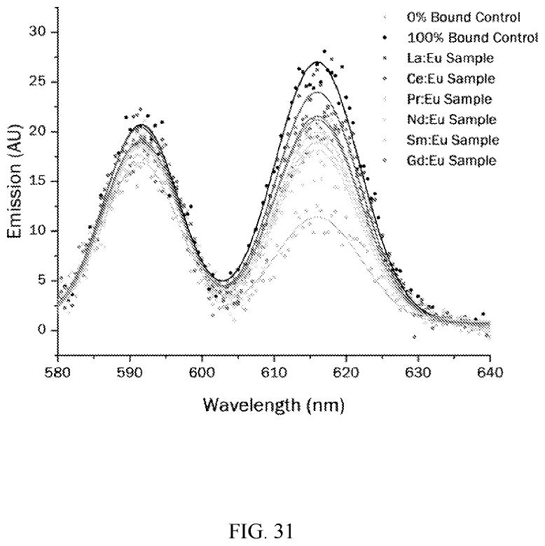

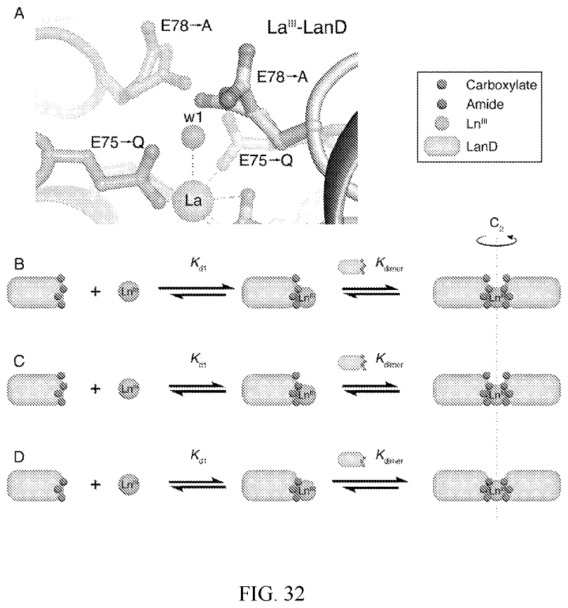

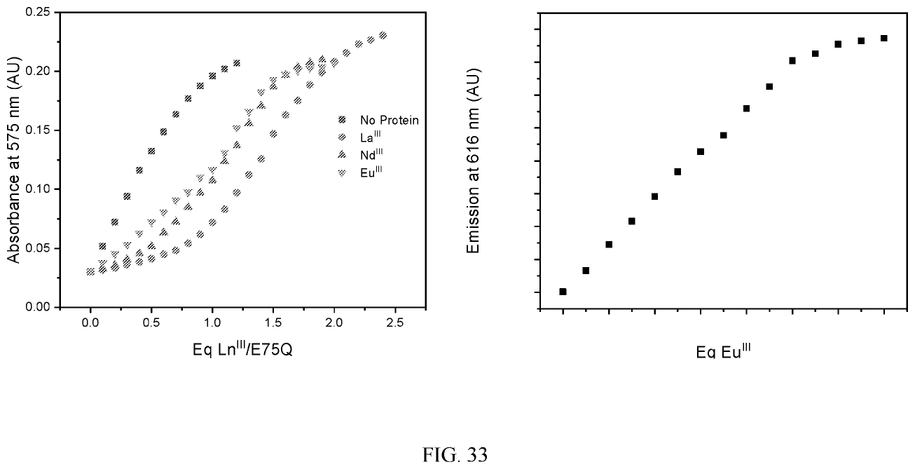

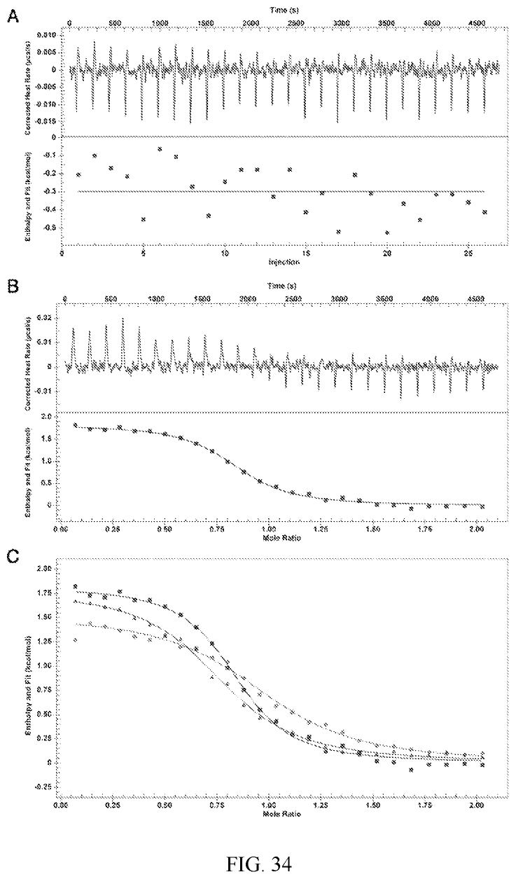

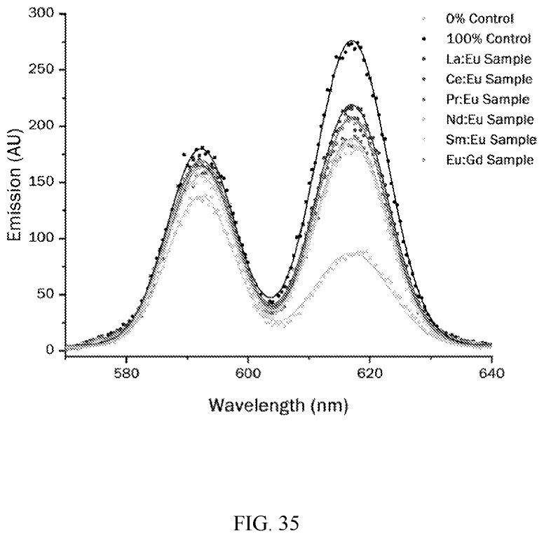

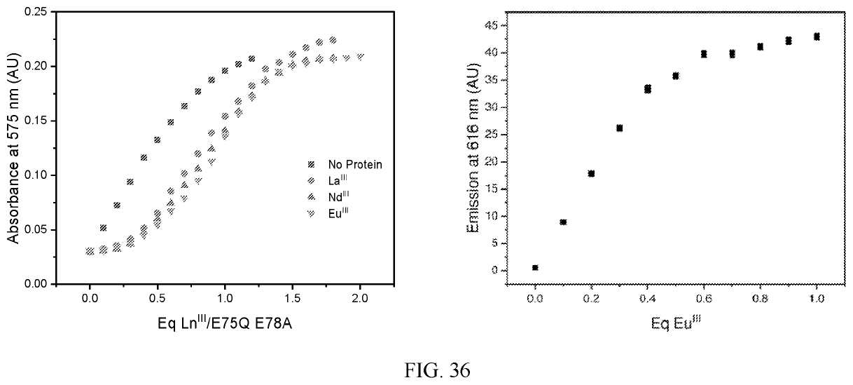

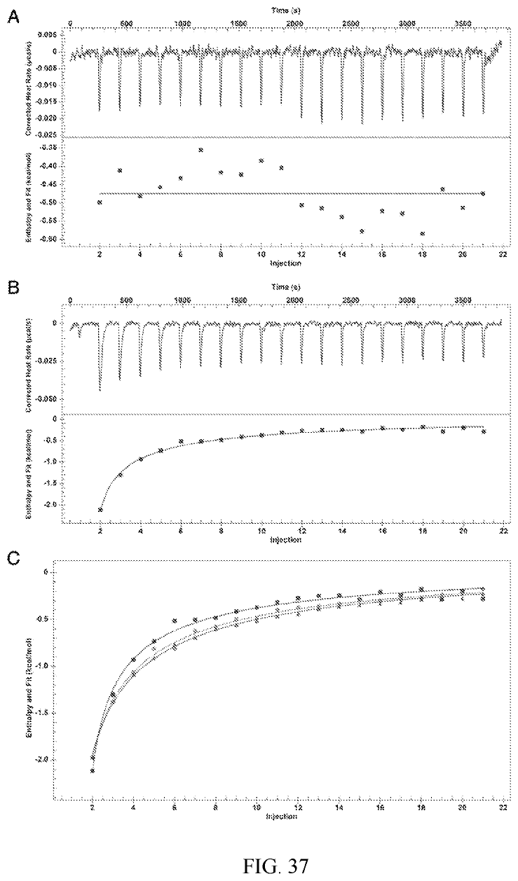

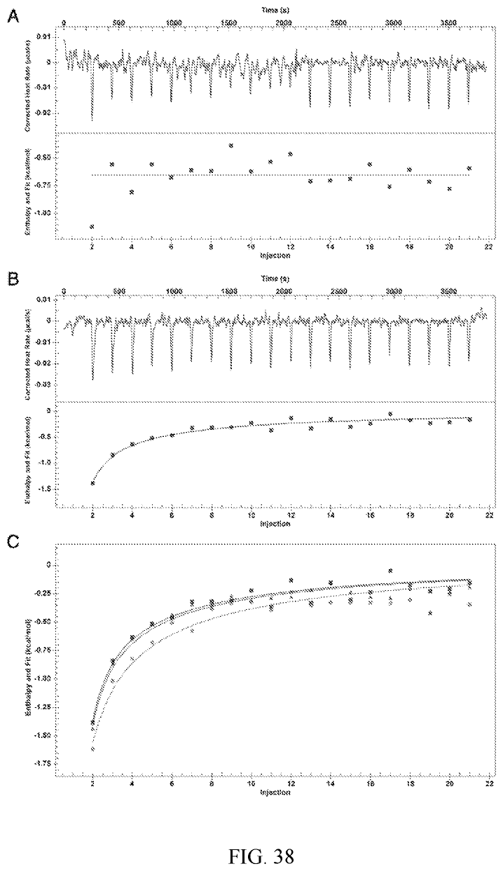

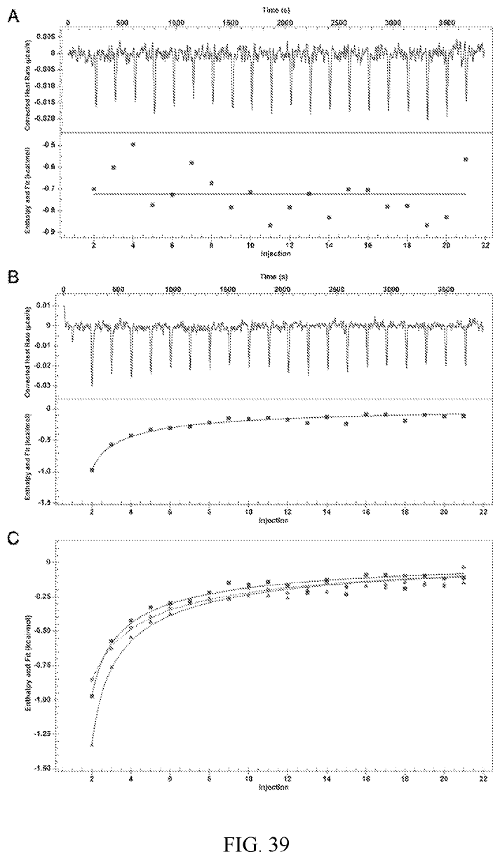

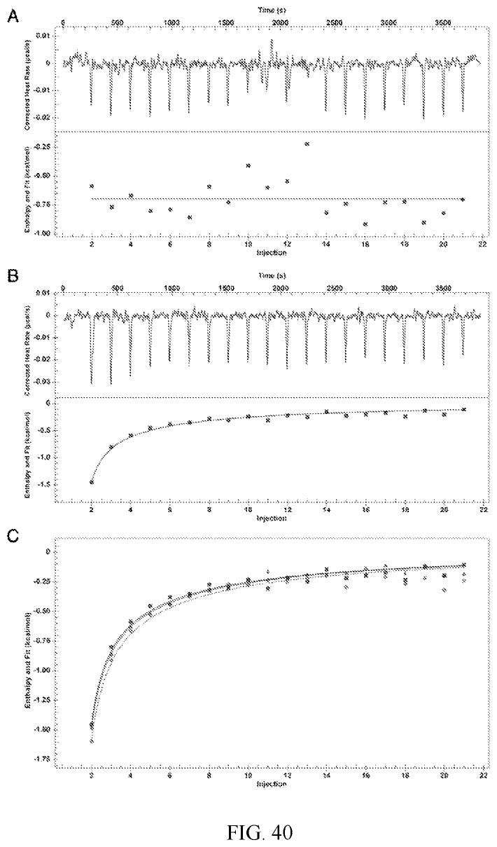

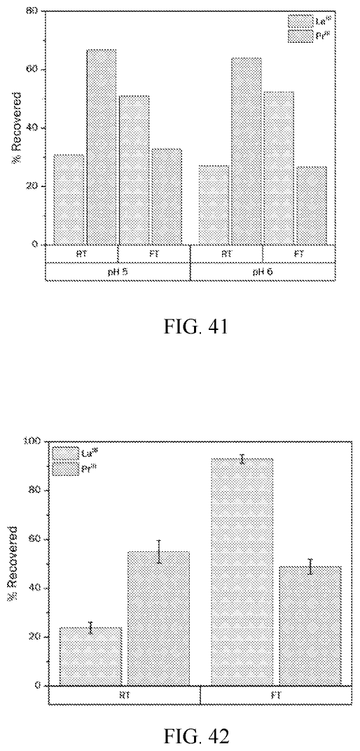

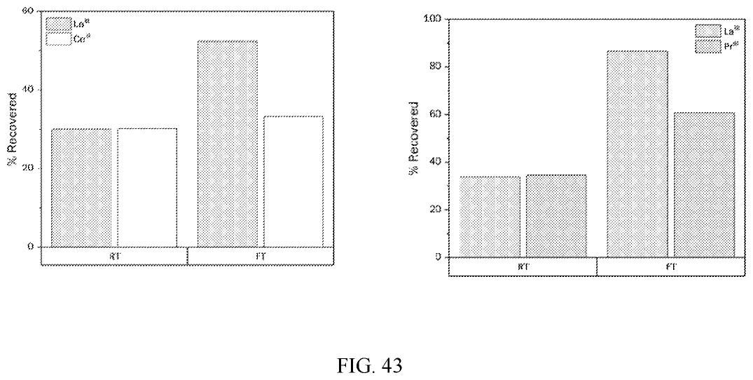

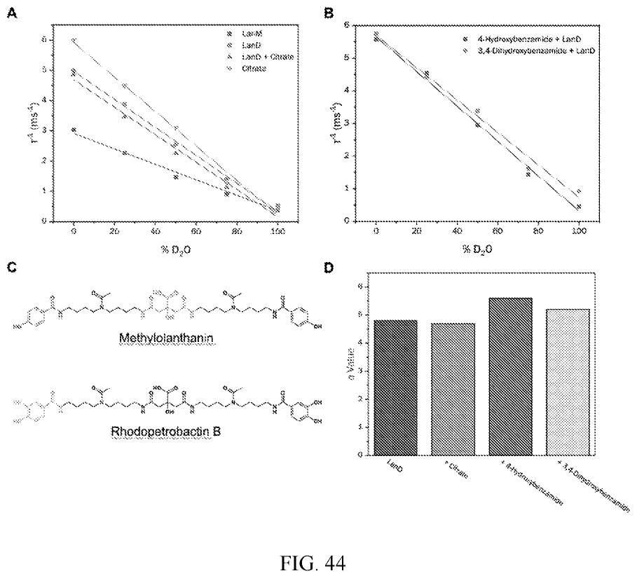

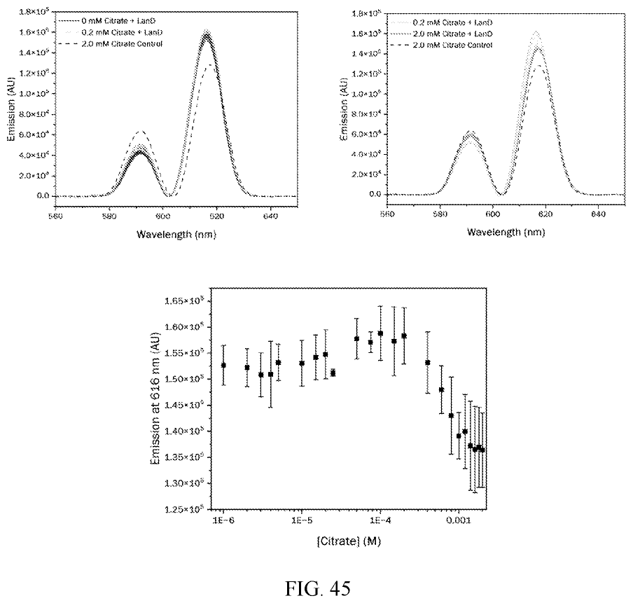

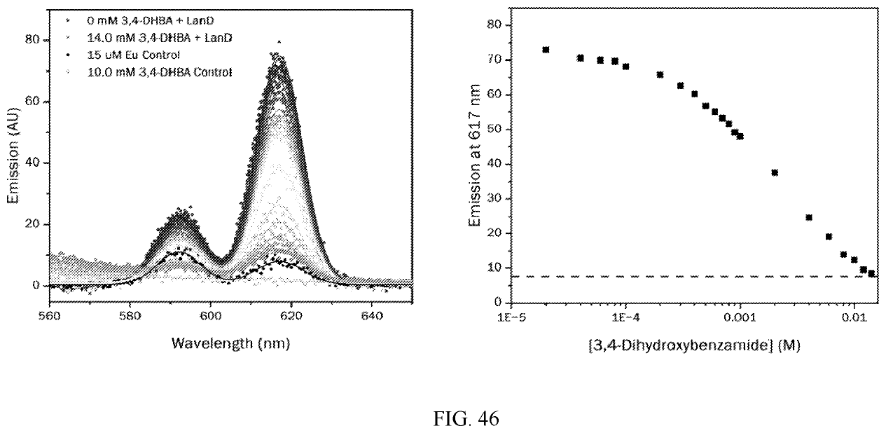

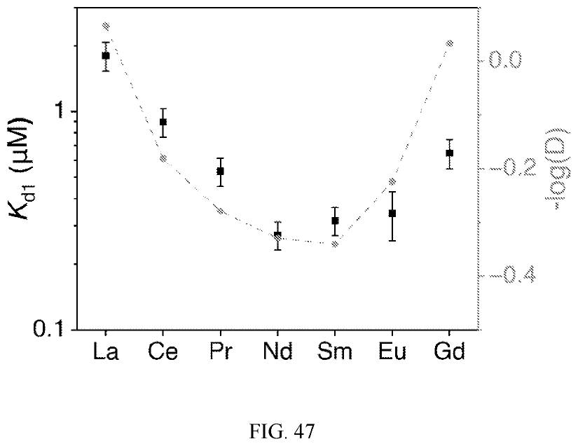

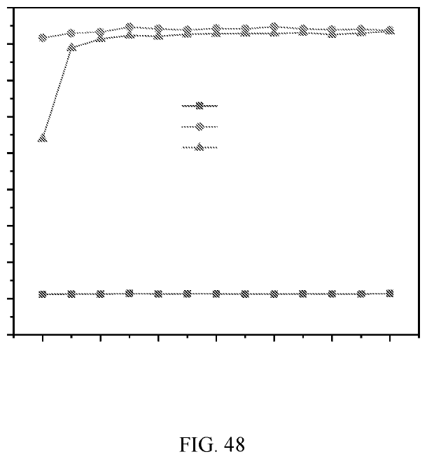

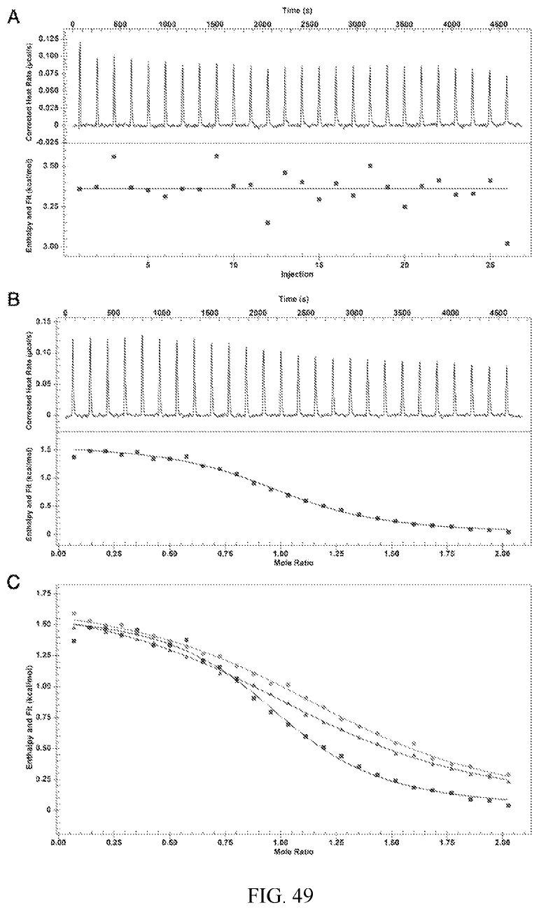

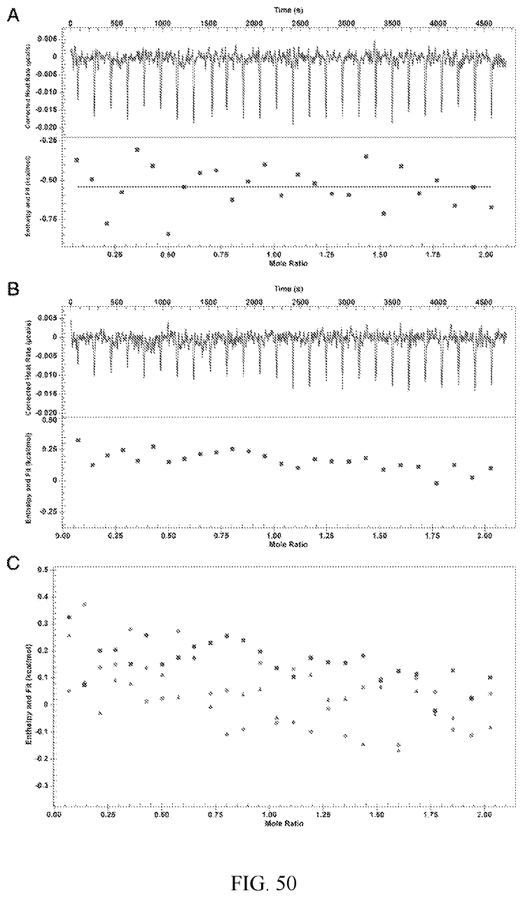

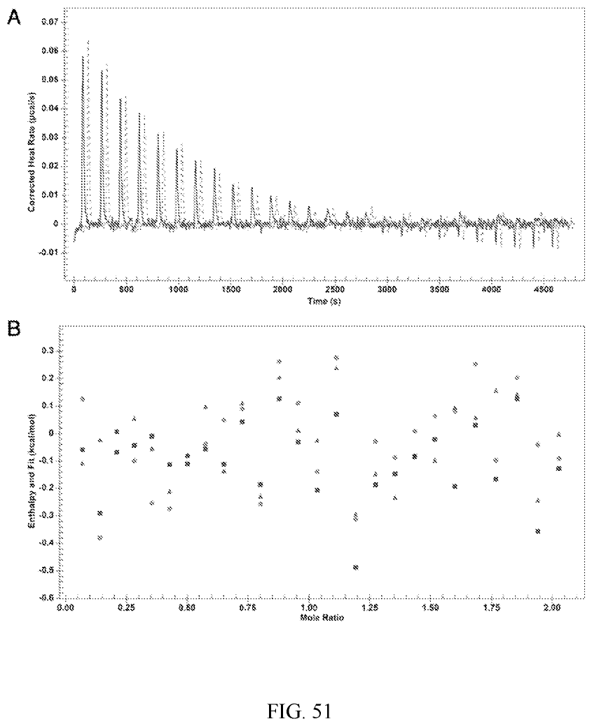

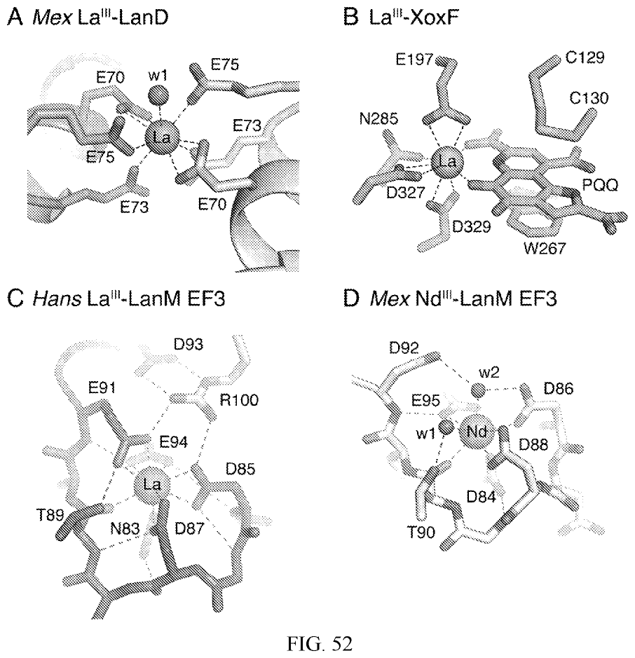

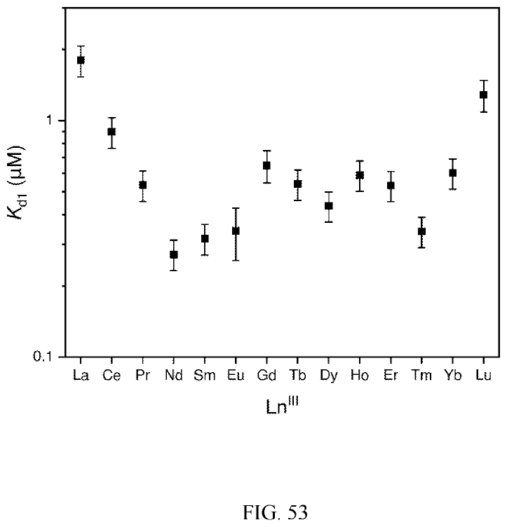

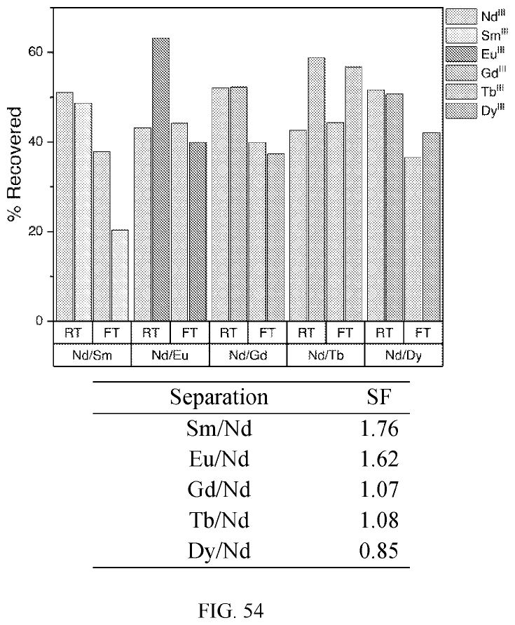

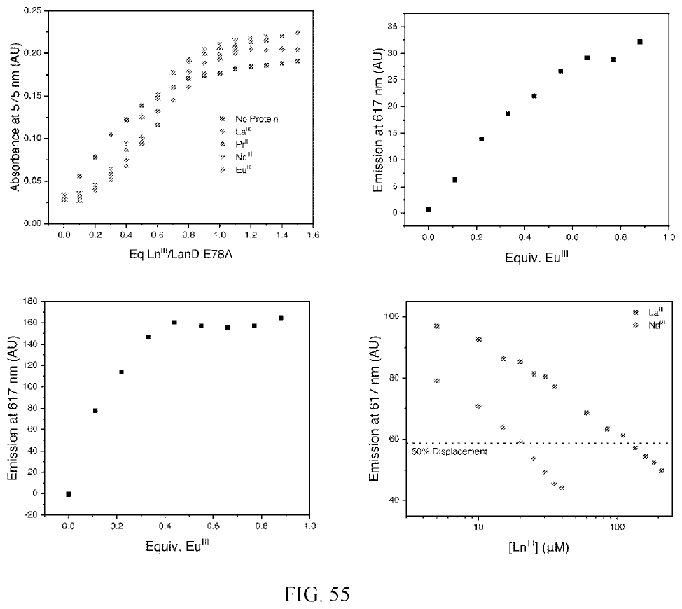

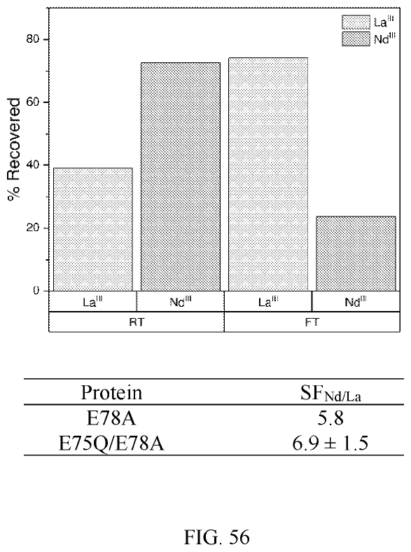

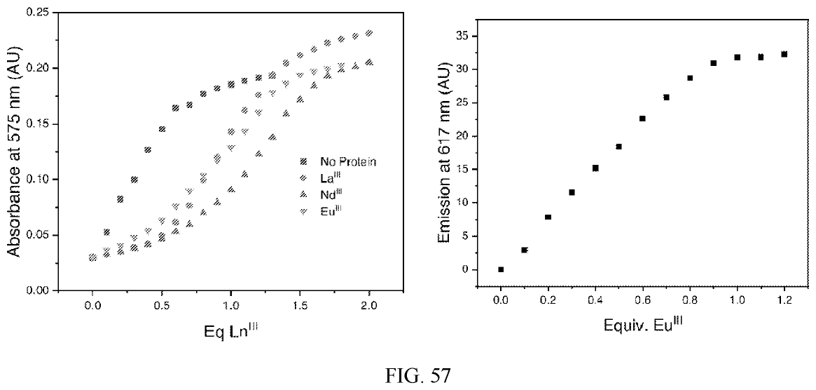

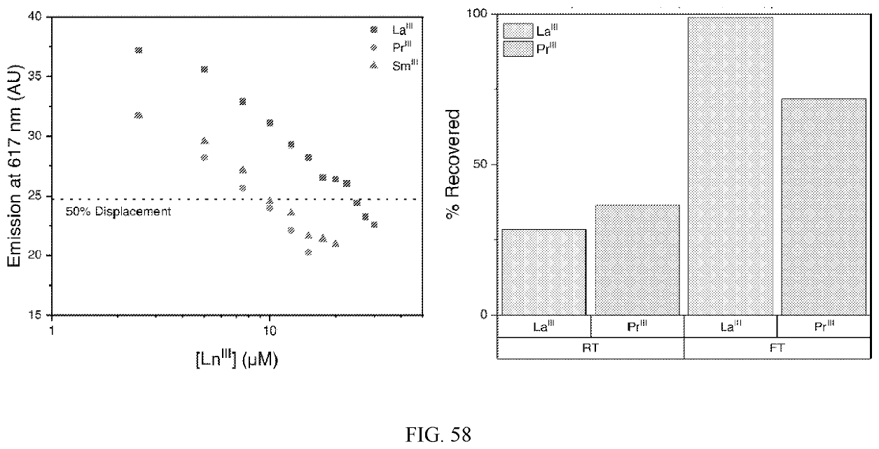

Inspection of the metal binding site in La^{III}-LanD (0.5 equiv) shows a symmetric arrangement of three glutamate ligands contributed by each monomer, providing eight coordination interactions (FIG. 4C). In each monomer, the central bidentate Glu70) is flanked by two monodentate glutamates. Glu73 and Glu75. The ligands project out toward the exterior of the protein from the C-terminal end of α2 and the transition to α3. The metal-binding Glu residues and several other carboxylates in the second sphere undergo conformational change to form the metal-binding site when compared to their counterparts in the apo structure (FIGS. 15-16). In La^{III}-LanD, a single water molecule, modeled at 0.5 occupancy, fills a ninth coordination site. Interestingly, LanD crystals contain only one monomer in the asymmetric unit, with the second half of the metal-linked dimer provided by a symmetry-related molecule in the crystal lattice, a phenomenon that underscores the C₂-symmetric arrangement of ligands. Analysis of the metal-centered dimer interface reveals that the coordination interactions nearly exclusively compose the dimer interface. The interface is not further stabilized by any significant hydrophobic contacts or hydrogen bonds involving other side chains. The lone exception is Arg69, which projects into the interface to stack against the monodentate ligand, Glu73, provided by the other monomer (FIG. 4D). This interaction may provide charge compensation for the unusual arrangement of the symmetric Glu73 ligands, in which a monodentate binding mode forces the non-coordinating side chain O atoms into very close proximity, only 2.5 Å apart. The Arg69 interaction is also symmetric, resulting in sandwiching of the Glu73 pair between the two second-sphere Arg side chains. The second sphere of the LanD metal binding site also exhibits an unusual number of flanking carboxylate side chains. Three additional Glu/Asp side chains (Asp77, Glu78, Glu81) cluster within ˜10 Å of the metal binding site, all contributed by α3. The residue most proximal to the La^{III} ion, Glu78, appears to adopt multiple conformations (FIG. 17). In one, the side chain projects close to the metal binding site, nearly overlapping with the coordinated water ligand. In the other conformer, the side chain is instead oriented away from the metal binding site. The multiple conformations of Glu78 and the presence of non-coordinated oxygen atoms with unsatisfied hydrogen bonding potential in ligands Glu73 and Glu75 could be consistent with a role in recognition of an exogenous ligand, such as another protein. Notably, the three metal ligands. Arg69, and Glu78 are among the few completely conserved residues in 263 LanD sequences predicted by BLAST (FIGS. 18-19), underscoring the significance of the metal binding site and its unusual second sphere.

### Dimer Affinity and Structure are Lanthanide Sensitive.

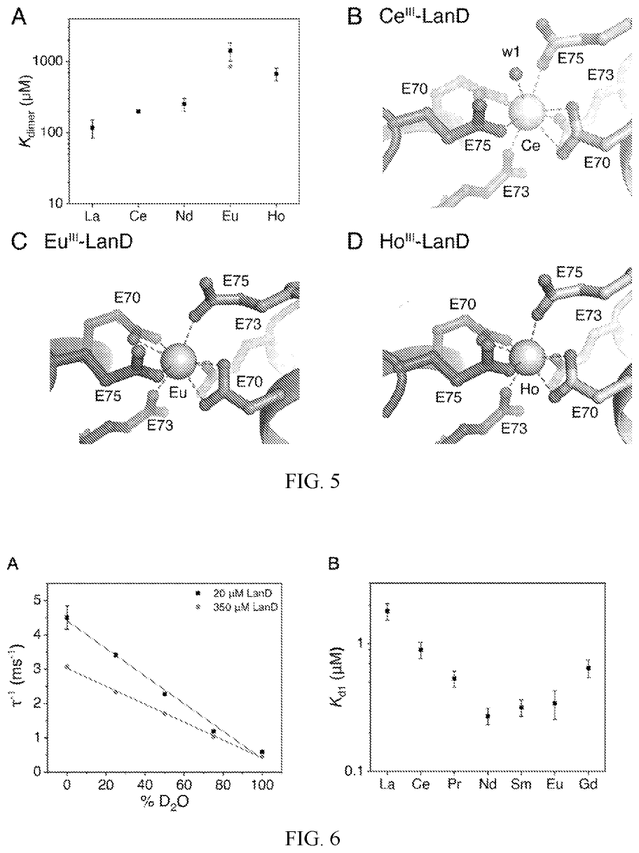

The crystallographic observation of both metal-independent and metal-centered dimerization motivated determination of the equilibrium constants for dimer dissociation (K_{dimer}) for LanD. Isothermal titration calorimetry was used, which is an approach previously applied to measure metal-dependent dimerization in Hans-LanM. Characterization of apo-LanD dimer dissociation shows an endothermic response, fitting to K_{dimer}=0.61 mM (FIG. 20, Table 5). K_{dimer} values for D39S and R47K variants were measured to be 0.80 and 1.06 mM, respectively (FIGS. 21-22, Table 5), supporting the relevance of these residues' interaction in the dimer in solution. In the case of the holoprotein, the K_{dimer} values increased as ionic radius decreased, from 120 μM for La^{III} to ˜1 mM for Eu^{III} and Ho^{III} (FIG. 5A: Table 6, FIGS. 23-27). The K_{dimer} values in the presence of Eu^{III} and Ho^{III} are endothermic and more similar to that of the apoprotein, suggesting that these ITC-determined K_{dimer} values may reflect both metal-dependent and metal-independent dimerization: nevertheless, the magnitude of the Eu^{III} K_{dimer} value is supported by luminescence studies (vide infra).

To investigate LanD's preference for the largest lanthanides in forming metal-centered dimers, we solved X-ray structures of the protein with Ce^{III}, Eu^{III}, and Ho^{III} all at ratios of 0.5 metal:protein. All exhibit the same symmetry-related metal-centered dimer observed in the La^{III}-LanD structure. Cell neighbors La^{III} on the periodic table and is most similar in size. The Ce^{III} binding site resembles the La^{III} binding site, including the exogenous solvent ligand (FIG. 5B). The most significant difference is a diminished occupancy for this water (0.35 in Ce^{III}-LanD versus 0.49 in La^{III}-LanD) (FIG. 28). This difference in water occupancy may reflect the smaller ionic radius of Ce^{III} that may not as readily accommodate a ninth ligand. Consistent with this prediction, structures of LanD with smaller lanthanides, Eu^{III} and Ho^{III}, show complete loss of the solvent ligand (FIGS. 5C, 5D). The increase in K_{dimer} with decreasing ionic radius appears to correlate with loss of the coordinated solvent molecule observed at partial occupancy in the structures of La^{III}- and Ce^{III}-LanD. We hypothesize that, as the ionic radius of the lanthanide ions contracts from La^{III} to Ho^{III}, increasing steric and charge repulsion between the multiple carboxylates at the dimer interface yields a smaller coordination number and favors dimer dissociation.

LanD Monomer Favors Nd^{III}, Sm^{III}, and Eu^{III} Binding.

Despite the intriguing self-dimerization phenomenon, the K_{dimer} values reported above likely are not tight enough to be relevant in the cell, which would leave LanD monomeric and the Ln^{III} ion coordination spheres only partially satisfied by protein ligands. To support this interpretation, the dependence of Eu^{III} luminescence lifetime on protein concentration was examined. The number of water molecules (q) in the first coordination sphere of Eu^{III} can be estimated based on its luminescence lifetime. Fully aquated Eu^{III} has 8-9 ligands (average of 8.3). At 20 μM LanD, where the monomer dominates and Eu^{III} is fully protein bound (FIG. 29), q=4.1, while at 350 μM, q decreases to 2.6 (FIG. 6A). Assuming that the holodimer has no coordinated solvent (q=0) based on the X-ray structure of Eu^{III}-LanD, at 350 μM LanD, ˜65% of the protein units are monomeric in solution, yielding K_{dimer}=850 μM for Eu^{III}-dependent dimerization. These results validate the interpretation of the ITC-derived K_{dimer} values, and they confirm that solvent provides approximately half of the coordination sphere for Eu^{III} bound to the LanD monomer.

Therefore, the metal affinities (K_{d1}) of the more physiologically relevant monomer were determined. The weak K_{dimer} value for Eu^{III} allowed use of ITC to determine K_{d1} for Eu^{III}-LanD to be 340 nM, with a stoichiometry of 1.0 (FIG. 30, Table 7). The relative K_{d1} values for other Ln^{III} ions were estimated by direct competition with Eu^{III}, taking advantage of the higher luminescence intensity of protein-bound vs, unbound Eu^{III} ion, and converted into absolute K_{d1} values using the ITC-determined K_{d1} for Eu^{III} (FIG. 6B: FIG. 31, Table 8). These values are substantially tighter than the K_{dimer} values and show an opposite trend in sensitivity to RE identity, with affinity increasing from La^{III} to Nd^{III}, plateauing, and then decreasing beyond Eu^{III}. Thus, the LanD monomer favors binding of lanthanide ions that are less preferred for supporting methylotrophic growth.

### Engineering LanD for Dimer-Mediated Separations.

The above structural and biochemical insights to were applied to separations. Although wild-type LanD's metal-centered dimerization is weak, it was envisioned that its interfacial metal site could be exploited by re-engineering LanD to dimerize selectively in the presence of higher-value Pr^{III} and Nd^{III} over La^{III} and Ce^{III}. It was reasoned that this goal would require weakening metal binding to monomer (K_{d1}) in general and tightening K_{dimer} selectively for Pr^{III} and Nd^{III}, which would likely involve overcoming steric constraints to preferentially stabilize an octacoordinate metal site (FIG. 32). This approach would make K_{dimer} for preferred elements tighter than K_{d1} for non-preferred elements, an arrangement fundamentally distinct from the dimerizing Hans-LanM system, where K_{d1} is much tighter (picomolar) than K_{dimer} (high nanomolar to low micromolar).

To weaken K_{d1}, one of the monodentate carboxylate ligands. Glu75, was mutated to Gln (E75Q) (FIG. 32). Competition assays against xylenol orange show qualitatively that K_{d1} values in E75Q are weaker than in wild-type LanD (Table 9, FIG. 33). ITC studies indicated a K_{d1} value for Eu^{III}-LanD-E75Q of 0.88 μM with n=1, indicating a monomer under these conditions, and a titration of 50 μM E75Q with Eu^{III} followed by luminescence yields 1:1 stoichiometry, suggesting that K_{dimer} is still substantially weaker than K_{d1} (FIG. 34). Luminescence competition experiments were used to determine the trend in K_{d1} values for La^{III} to Gd^{III}, which is similar to that of wild-type LanD (FIG. 7A; FIG. 35).

It was reasoned that removing the steric and charge repulsion near the metal site arising from the outer-sphere residue that occupies two conformations in the X-ray structures, Glu78, might strengthen dimerization, particularly for smaller lanthanide ions. An E78A variant was constructed in the E75Q background. Competition assays with xylenol orange were consistent with stoichiometries of 0.5, suggesting substantial dimerization under the experimental conditions (10 μM protein) and therefore that K_{dimer} is now in the low micromolar range (FIG. 36). Indeed, time-resolved luminescence titration of 10 μM LanD-E75Q/E78A with Eu^{III} showed an endpoint at 0.5 equivalents. Competitive titrations of this presumptive Eu^{III}-bridged dimer showed that La^{III} and Ce^{III} competed poorly—with 50-60 μM of these metal ions required to outcompete Eu^{III} binding by 50% (resulting in a decrease of Eu^{III} luminescence)—whereas ˜20 μM Pr^{III} and ˜10 μM Nd^{III} were required (FIG. 7B). The K_{dimer} values were measured by ITC for the complexes with La^{III}-Nd^{III} (FIGS. 37-40), validating this result (FIG. 7A, Table 10). K_{d1} s could not be measured by ITC because K_{dimer}'s are on the order of typical protein concentrations, but it is proposed that they may be similar to those with E75Q. Therefore, we believe we have achieved K_{dimer} in the range of K_{d1}. Remarkably, from these two substitutions, the affinities of the Ln^{III}-induced dimers are increased by 10- to 100-fold compared to the wild-type LanD. This pattern shows that large selectivity effects can be achieved from even simple substitutions at the LanD interface. These results led to investigation on the ability of LanD-E75Q/E78A to separate light lanthanides, La^{III}-Nd^{III}, from one another. Spin concentrators with a 10-kDa cutoff membrane were used for small-scale separation tests with pairs of Ln^{III} ions, envisioning that Nd^{III} and Pr^{III} would preferentially induce dimerization (˜14 kDa) and would be less likely to flow through the filter. The separation factors (SFs) were determined from the ratios of the distribution coefficients of each metal between retentate and flowthrough, pH 5 and 6 and varied starting protein concentration and metal:protein stoichiometry (FIGS. 41-42) were tested and it was found that 3:1 monomer: target metal (Pr^{III} or Nd^{III}) yielded the best SFs. Wild-type LanD has poor SFs (FIG. 43). LanD-E75Q/E78A, however, achieved up to 70-80% recovery of Pr^{III} and Nd^{III} in the retentate and 60-80% partitioning of La^{III} to flow through (FIG. 7C). When equal concentrations of La^{III}, Ce^{III}, Pr^{III}, and Nd^{III} were used together, the SFs were similar to those obtained in binary element experiments (Table 3; FIG. 7D; Table 11). These SFs are higher than for common industrial extractants DEHPA and PC88A. Advantageously, the entire LanD process of incubation and filtration takes <1 h, as opposed to many synthetic ligands for which SFs are reported at 24 h.

TABLE 3
Separation factors for LanD-E75Q/E78A (5 μM), filtration, separation
of mixture of 0.8 μM each La^{III}, Ce^{III}, Pr^{III}, Nd^{III}.

| | La^{III} | Ce^{III} | Pr^{III} | Nd^{III} |
|---|---|---|---|---|
| La^{III} | 1 | 3.0 ± 0.4 | 5.1 ± 0.6 | 7.3 ± 0.9 |
| Ce^{III} | | 1 | 1.7 ± 0.2 | 2.4 ± 0.3 |
| Pr^{III} | | | 1 | 1.4 ± 0.2 |
| Nd^{III} | | | | 1 |

LanD Interacts with Apo-LanM.

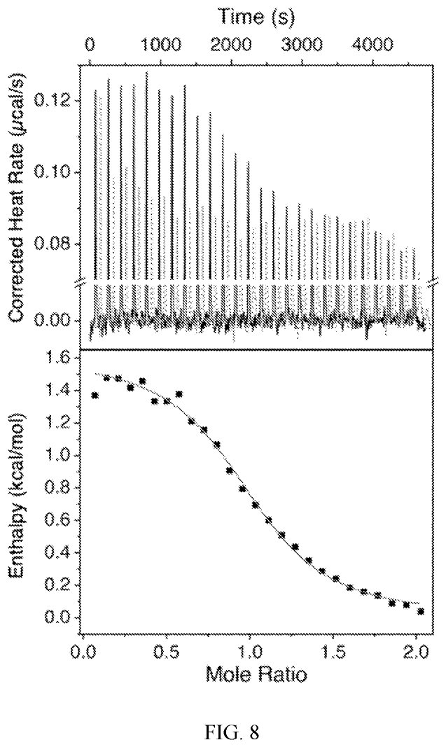

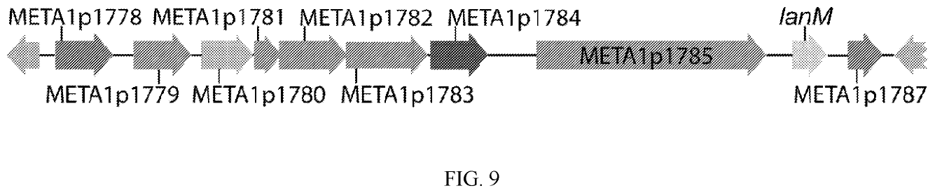

The observation of a higher-affinity dimer in LanD-E75Q/E78A reinforces the notion that the conserved, highly negatively charged environment of the metal site serves to disfavor dimerization in the wild-type protein. Therefore, we sought to obtain insight into LanD's biological function in light of this unusual surface metal site. We first considered the possibility that the coordination sphere of a LanD-bound Ln^{III} ion might be completed in a ternary complex with another multidentate ligand. We investigated several chelators of potential in vivo periplasmic relevance and found no evidence of ternary complex formation (see below. FIG. 44-46). Therefore, it was considered that the surface site might enable rapid transfer of Ln^{III} ions between LanD and other periplasmic proteins encoded by the lanthanide uptake gene cluster. The similarity in affinity trends of LanD's K_{d1} values and K_{d,app} values of LanM (FIG. 6B: FIG. 47) motivated investigation of a potential LanD-LanM interaction. Mixing of La^{III}-LanD and LanM (in the form of the Ln^{III}-responsive fluorescent sensor, LaMP1) shows rapid transfer of La^{III} to LanM (FIG. 48). Because LanD's K_{d1} values are 10⁵- to 10⁶-fold weaker than those of LanM, however, this result does not necessarily indicate direct transfer. The interaction of apo-LanM with apo-LanD was examined using ITC. Apo-LanD was used rather than holo-LanD to avoid large heats associated with LanM metalation and because the structures of apo- and holo-LanD are similar. The two proteins interact with K_{d}=4.0±1.9 μM and 1:1 stoichiometry (n=1.2±0.2), parameters that suggested a physiologically relevant interaction (FIG. 8; FIG. 49, Table 12).

If LanD were to transfer Ln^{III} ions to LanM inside the cell, one would expect LanD may not interact as tightly with Ln^{III}-bound LanM. Indeed, titration of apo-LanD with Sm^{III}₃-LanM (Sm^{III} being favored by both LanM and LanD) shows no evidence of interaction (FIG. 50). Because apo-LanMs characterized to date are primarily intrinsically disordered and therefore might be able to complex non-specifically with other proteins, we titrated apo-Mex-LanD with apo-Hans-LanM, which also provided no evidence of interaction (FIG. 51). The specific interaction of LanD with apo-Mex-LanM suggests that LanM is the exogenous ligand that the Ln^{III}-LanD site recognizes in vivo, with the function of that recognition being transfer of Ln^{III} ions from LanD to LanM. This model implies a chaperone function for LanD.

M. extorquens LanD is only the third class of biological lanthanide-binding site to be structurally characterized, after the Ln-dependent alcohol dehydrogenases and lanmodulins. Unlike these previously crystallographically characterized sites. LanD is structurally unrelated to known biological ligands for Ca^{II}. Three residues, one bidentate carboxylate flanked by two monodentate carboxylates, provide just four of the requisite eight to nine ligands for the bound lanthanide ion. The LanD metal-binding site does not allow for coordination from backbone atoms (unlike in LanMs; FIG. 52) and, as a result of the coordination environment not being saturated by ligands from a monomeric unit, a face-to-face arrangement of carboxylates from two protomers is observed in the crystal structure of the metal-bridged dimer. The excessive negative charge of the dimeric metal site is enhanced by several additional nearby negatively charged residues, including the second-sphere, conserved residue Glu78, implicated in destabilizing the metal-dependent dimer interface. The importance of charge at this interface is reinforced by the strong enhancement of K_{dimer} induced by the inner-sphere E75Q and outer-sphere E78A substitutions. Because Ln^{III} ions favor high coordination numbers, a surface site only half-coordinated by protein residues would be prone to self-dimerization with another protein monomer, but it was proposed that the charge repulsion of wild-type LanD metal site serves to disfavor this process.

LanD's C₂-symmetric dimer centered on a single metal ion is a relatively simple scaffold from which to design metal sites that can discern between elements by exploiting differences in ionic radius, hydration, and coordination number, as our separations work demonstrates. As a single ligand. LanD's aqueous SFs are higher than common extractants DEHPA and PC88A and comparable to next-generation diglycolamide extractants, typically implemented in liquid-liquid extraction schemes, where the lanthanide ion partitions between an organic phase with an organic extractant and an aqueous phase (Table 13). They are also similar to other dimerizing ligand systems, such as a supramolecular encapsulation approach (SF_{Nd/La}=6) that is conceptually similar to LanD's metal-centered dimerization, and the dimerizing TriNOx ligands (SF_{La/Nd}˜10). LanD's SFs are lower than the “tug-of-war” systems using two or more chelators, particularly macrophosphi, which has the highest SF for adjacent lanthanides, although they are more similar after accounting for the contribution of the other chelators present (DEHPA and lactic acid). However, the recent LanM-based column systems, which are also single-ligand and all-aqueous, may be more appropriate points of direct comparison than two-phase, multi-ligand systems. LanD's SFs substantially outperform both the original Mex-LanM column and the improved Hans-LanM column in the La-Nd range (Table 13), and LanD's weaker metal binding under milder conditions could also be favorable for rapid separations. The small number of inter-monomer interactions beyond LanD's metal-binding site suggests that this interface could be engineered to further amplify dimer affinity and RE/RE selectivity, as well as shift selectivity trends to access separations of smaller REs. Tethering of the dimers together (covalently or non-covalently) and immobilization on a column or porous membrane could yield sterically congested metal sites that would strongly disfavor binding of La^{III} and Ce^{III}. Furthermore, the rigid, disulfide bridged structure of LanD may be a desirable candidate for simplification to a cyclic peptide.

The surprising properties of LanD, in particular surface accessibility and affinity trends of its metal site, also provided insights into lanthanide trafficking in the cell. leading to experiments strongly suggesting that a physiological function of LanD is to transfer Ln^{III} ions to LanM. The interaction and directional transfer of Ln^{III} ions between these proteins restricts the possible mechanisms of lanthanide trafficking in the periplasm given the other activities encoded in the cluster. In particular, the observation that both proteins prefer not the biologically preferred La^{III} and Ce^{III} but rather Nd^{III} and Sm^{III}—which are less favored in biology but still abundant in the environment and therefore need to be withheld from lanthanide-dependent enzymes—is crucial. Outer-membrane uptake of Ln^{III} ions via a presumptive Ln^{III}-metallophore complex is promiscuous, necessitating an additional source of selectivity to account for the sharp and nearly complete cutoff in cytosolic uptake between Nd^{III} and Sm^{III}.

A pathway with selectivity opposite that of lanthanide preference of enzyme metalation would fit the bill for being able to “siphon” off the less-desirable lanthanides, which incidentally would mean that Ln^{III} ions must be released from the metallophore in the periplasm so that sorting can take place. We propose that the LanD-LanM axis is (part of) this siphon, leaving the larger REs to be imported to the cytosol and the smaller REs transferred from LanD to LanM for sequestration in the periplasm or, possibly, export. This model would explain why the Beijerinckiaceae equivalents of lanD and lanM are upregulated to a greater extent in the presence of Nd^{III} than of La^{III}. The functional connectivity between LanD and LanM is also supported by the observation that in the 263 organisms in which LanD orthologs were identified by BLAST search (FIG. 18). LanM orthologs in all but 6 (and 3 of those had LanMs annotated in organisms in the same genus) were identified. It was suggested that the other ˜450 LanMs identified to date may have LanD equivalents that are structurally distinct from M. extorquens LanD but fulfill a similar function (perhaps with different metal selectivities). Although LanD and LanM are not required for growth on La^{III}, this proposal predicts that both might be particularly important in the presence of non-preferred REs such as Sm^{III}.

### MATERIALS AND METHODS

The expression, purification, and in vitro characterization of wild-type LanD and its E75Q and E75Q/E78A variants are described below. This information includes methods and data for crystallographic structure determination of apo-, La^{III}-, Ce^{III}, Eu^{III}, and Ho^{III}-LanD. Below provides detailed methods, chromatograms, spectra, and full thermodynamic parameters derived from ITC- and luminescence-based metal and protein titration experiments for K_{dimer} and K_{d1} determinations for LanD and its variants, as well as for studies of the LanD-LanM interaction. Methods and supporting data for LanD-based separation experiments are also described. Finally, the below also includes methods and data for luminescence-based titrations to assess potential ternary complex formation in LanD with small molecules, the results of which are also described below:

### GENERAL CONSIDERATIONS

Chemical reagents were obtained from Millipore Sigma unless otherwise noted. All lanthanide (III) chloride salts were at a minimum purity of 99.9% rare earth metal content. Stock solutions of Ln^{III} ions were prepared by dissolution in 1 M HCl to achieve ˜0.5 M solution and their concentration was determined by inductively coupled plasma mass spectrometry (ICP-MS) analysis on a Thermo Scientific iCAP RQ instrument with He in KED mode, in the Laboratory for Isotopes and Metals in the Environment (LIME) at the Pennsylvania State University. E, coli BL21 (DE3) (for recombinant protein expression) was obtained from New England Biolabs. Plasmids for expression and purification of proteins (pET29b-based vectors) were obtained from Twist Bioscience. M. extorquens LanM, LaMP1, and H. quercus LanM were expressed and purified as described in our previous work. Q Sepharose and Phenyl Sepharose Fast Flow resins were obtained from Millipore Sigma. Automated protein chromatography was carried out on a GE Healthcare Biosciences Akta Pure fast protein liquid chromatography (FPLC) system using either a HiLoad Superdex 75 μg 16/600 column for preparative scale or a Superdex 75 pg Increase 10/300 GL column for analytical scale. UV-visible absorption spectra were obtained on an Agilent Cary 60 UV-visible spectrophotometer using a quart/cuvette (Starna Cells). Fluorescence emission spectra were obtained using a Horiba Fluorolog-QM fluorometer equipped with a double monochromator on the excitation arm and single monochromator on the emission arm. A quartz cuvette with 10 mm×2 mm dimensions (Starna Cells, Inc.) was used. Isothermal titration calorimetry (ITC) and SEC-MALS measurements were carried out at the X-ray Crystallography and Automated Biological calorimetry Facility at Penn State.

### Expression and Purification of LanD and its Variants.

M. extorquens LanD was expressed from pET24a-p1781, which was described previously by our group (Mattocks et al., JACS 2019). This construct contains a C-terminal Tyr-Gly addition (to facilitate protein quantification as the native protein contains no Tyr or Trp residues). Electrocompetent E, coli BL21 (DE3) cells were transformed with pET24a-LanD and plated on LB-agar plates containing 50 μg/ml, kanamycin (Km) and incubated at 37° C. A single colony was used to inoculate 200 mL of LB (50 μg/mL Km in all growth media), which was grown for ˜16 h at 37° C. with shaking at 200 rpm. This culture was used to inoculate three 2 L cultures (in 6 L flasks). The cultures were grown at 37° C. with shaking at 170 rpm. At OD₆₀₀ₙₘ˜0.6, isopropyl-β-D-thiogalactopyranoside (IPTG, Oakwood Chemical) was added to a final concentration of 2 μM and the cultures were further incubated at 20° C. for ˜16 h. (The lower concentration of IPTG is an improvement of the previously published protocol, and it leads to substantially higher yields of purified protein.) The cells were pelleted by centrifugation for 7 min at 7000×g at 4° C., yielding ˜5 g cell paste per L culture. The periplasmic extract was prepared using the cold osmotic shock method, as described (2). The periplasmic extract in 5 mM MgSO₄ was buffered by addition of 0.05 volumes of 1 M Tris, pH 7.4 and filtered through a 0.2 μm polyethersulfone (PES) membrane. This solution was applied to a 2.5×4.5 cm (20 mL) Q-Sepharose Fast Flow column that had been pre-equilibrated in 50 mM Tris, 1 mM EDTA, pH 7.0 (Buffer A). The column was washed with 2 CV Buffer A, and the protein was eluted with 5 CV Buffer A containing 50 mM NaCl and 5 CV Buffer A containing 100 mM NaCl. LanD-containing fractions were determined by SDS-PAGE gel analysis. The column wash and elution fractions were concentrated to 20 mL and exchanged into 50 mM Tris, 2.5 M NaCl, pH 8.0 buffer (Buffer B) using an Amicon Ultra-15 3-kDa MWCO centrifugal filter. This solution was applied to a 2.5×4.5 cm (20 mL) Phenyl Sepharose column that had been pre-equilibrated in Buffer B. The column was washed with 1 CV Buffer B. Flowthrough and wash fractions were concentrated to 5 mL and buffer exchanged into 20 mM MES, 100 mM KCl, 5 mM acetate, pH 6.0 buffer (Buffer C) using an Amicon Ultra-15 3-kDa MWCO centrifugal filter. LanD was separated from higher molecular weight proteins by size-exclusion chromatography on a HiLoad 16/600 Superdex 75 pg column. The protein sample was loaded onto the column using a 5-mL capillary loop and eluted with 1.2 CV Buffer C. Fractions (2 mL) were collected in peak fractionation mode with a 1 mAU threshold at 280 nm. LanD eluted at 73-80 mL. LanD-containing fractions were dialyzed against 20 mM Tris, 100 mM KCl, pH 7.0 containing Chelex-100 as described for LanM. The extinction coefficient of LanD (with the C-terminal YG addition) was determined to be ε₂₇₅ₙₘ=1430 M⁻¹ cm⁻¹ by correlation of UV-visible absorption spectra and Direct Detect measurements (Automated Biological calorimetry Facility). The purification yielded 14 mg LanD per L culture.

The gene encoding M. extorquens LanD, codon optimized for expression in E. coli with the D39S or R47K mutation and a C-terminal YG addition, was obtained from Twist Bioscience inserted into the NdeI/XhoI sites of pET-29b(+). Expression and purification of these variants was conducted using the same procedure as above, yielding 10 mg per L culture (D39S) and 11 mg per L culture (R47K).

The gene encoding M. extorquens LanD, codon optimized for expression in E. coli with the E75Q or E75Q/E78A mutation and a C-terminal YG addition, was obtained from Twist Bioscience inserted into the NdeI/XhoI sites of pET-29b (+). Expression and purification were conducted using the same procedure as above with modification to the buffers used for the Q-Sepharose Fast Flow column. For E75Q, the periplasmic extract in 5 mM MgSO₄ was buffered by addition of 0.05 volumes of 1 M Tris, pH 7.4 and filtered through a 0.2 μm polyethersulfone (PES) membrane. This solution was applied to a 2.5×4.5 cm (20 mL) Q-Sepharose Fast Flow column that had been pre-equilibrated in 50 mM Tris, 1 mM EDTA, pH 8.0 (Buffer D). The column was washed with 2 CV Buffer D, and the protein was eluted with 5 CV Buffer D containing 50 mM NaCl and 5 CV Buffer D containing 100 mM NaCl. E75Q-containing fractions were determined by SDS-PAGE gel analysis. For E75Q/E78A, the periplasmic extract in 5 mM MgSO₄ was buffered by addition of 0.1 volumes of 360 mM CAPS, pH 11 and filtered through a 0.2 μm polyethersulfone (PES) membrane. This solution was applied to a 2.5×4.5 cm (20 mL) Q-Sepharose Fast Flow column that had been pre-equilibrated in 50 mM CAPS, 1 mM EDTA, pH 11.0 (Buffer E). The column was washed with 2 CV Buffer E, and the protein was eluted with 5 CV Buffer D containing 100 mM NaCl and 5 CV Buffer D containing 200 mM NaCl. LanD-E75Q/E78A-containing fractions were determined by SDS-PAGE gel analysis. Purification of these variants yielding 14 mg per L culture (E75Q) and 25 mg per L culture (E75Q/E78A). Amino acid sequences of all proteins used are presented in Table 14.

### La^{III}-LanD X-Ray Crystallography Data Collection and Structure Determination.

Purified M. extorquens LanD was exchanged into 30 mM MOPS and 50 mM KCl, pH 7, prior to crystallization. The protein was loaded with 0.5 equivalents of La^{III} (LaCl₃) per monomer. Crystals were obtained by using the sitting drop vapor diffusion method, in which 1 μL of protein solution (19.3 mg/mL) was mixed with 1 μL 100 mM MES, pH 6.5, and 50% (w/v) PEG 200 in a 24-well plate from Hampton Research (cat, no. HR1-002) at room temperature. Cubic-shaped crystals appeared in one month. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Life Sciences Collaborative Access Team (LS-CAT) ID-G beamline and processed with the HKL2000 package. La^{III}-bound LanD crystallized in the I2₁3 space group with 1 monomer in the ASU. The structure was solved using the single-wavelength anomalous diffraction (SAD) method. Using a dataset collected at 12.7 keV (0.9792 Å), HySS identified one La^{III} site that was subsequently used to obtain phases in phenix.autosol. The initial figure of merit (FOM) and Bayesian CC were 0.442 and 0.52, respectively. An initial model was generated with phenix.autobuild with subsequent rounds of manual modification and refinement in Coot and phenix.refine. In the final stages of model refinement, anisotropic displacement parameters (ADP) were refined for all La^{III} sites. The final refinement afforded an FOM of 0.81 and R_{work}/R_{free} of 0.205/0.236. The final model consists of residues 32-91 in each chain, one La^{III} ion, and 50 water molecules. Of the residues modeled. 100% are in allowed or preferred regions as indicated by Ramachandran statistical analysis. Model validation was performed with the Molprobity server. Figures were prepared using the PyMOL molecular graphics software package (Schrödinger. LLC).

### Ce^{III}-LanD X-Ray Crystallography Data Collection and Structure Determination.

Purified Mex LanD was exchanged into 30 mM MOPS and 50 mM KCl, pH 7 prior to crystallization. The protein was loaded with 0.5 equivalents of Ce (CeCl₃) per monomer. Crystals were obtained by using the sitting drop vapor diffusion method, in which 1 μL of protein solution (7.4 mg/mL) was mixed with 1 μL 100 mM MES, pH 6.5, and 50% (w/v) PEG 200 in a 24-well plate from Hampton Research (cat, no. HR1-002) at room temperature. Cubic-shaped crystals appeared in one week. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Stanford Synchrotron Radiation Lightsource (SSRL) 12-1 beamline and processed with the XDS. Ce^{III}-loaded Mex LanD crystallized in the I2₁3 space group with 1 monomer in the ASU. The structure was solved using the molecular replacement in PHENIX. The coordinates of La^{III}-bound LanD were used as a search model after deletion of metal ions and water molecules. The final refinement afforded an R_{work}/R_{free} of 0.187/0.246. The final model consists of residues 32-91, one Ce^{III} ion, and 37 water molecules. Of the residues modeled. 100% are in allowed or preferred regions as indicated by Ramachandran statistical analysis. Model validation was performed with the Molprobity server. Figures were prepared using the PyMOL molecular graphics software package (Schrödinger, LLC).

### Eu^{III}-LanD X-Ray Crystallography Data Collection and Structure Determination.

Purified M. extorquens LanD was exchanged into 30 mM MOPS and 50 mM KCl, pH 7, prior to crystallization. The protein was loaded with 0.5 equivalents of Eu^{III} (EuCl₃) per monomer. Crystals were obtained by using the sitting drop vapor diffusion method, in which 1 μL of protein solution (19.3 mg/mL) was mixed with 1 μL 100 mM MES. pH 6.5, and 25% (w/v) PEG 200 in a 24-well plate from Hampton Research (cat, no. HR1-002) at room temperature. Cubic-shaped crystals appeared in one month. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Brookhaven National laboratory (BNL) 17-ID-1 AMX beamline and processed with the HKL2000 package. Eu^{III}-bound LanD crystallized in the I2₁3 space group with 1 monomer in the ASU. The structure was solved using the single-wavelength anomalous diffraction (SAD) method. Using a dataset collected at 13.5 keV (0.9201 Å). HySS identified one Eu^{III} site that was subsequently used to obtain phases in phenix, autosol. The initial figure of merit (FOM) and Bayesian CC were 0.638 and 0.58, respectively. An initial model was generated with phenix.autobuild with subsequent rounds of manual modification and refinement in Coot and phenix.refine. In the final stages of model refinement, anisotropic displacement parameters (ADP) were refined for all Eu^{III} sites. The final refinement afforded an FOM of 0.82 and R_{work}/R_{free} of 0.203/0.229. The final model consists of residues 32-91, one Eu^{III} ion, and 28 water molecules. Of the residues modeled. 100% are in allowed or preferred regions as indicated by Ramachandran statistical analysis. Model validation was performed with the Molprobity server. Figures were prepared using the PyMOL, molecular graphics software package (Schrödinger. LLC).

Ho^{III}-LanD X-Ray Crystallography Data Collection and Structure determination.

Purified M. extorquens LanD was exchanged into 30 mM MOPS and 50 mM KCl, pH 7, prior to crystallization. The protein was loaded with 0.5 equivalents of Ho^{III} (HoCl₃) per monomer. Crystals were obtained by using the sitting drop vapor diffusion method, in which 1 μL of protein solution (16.1 mg/mL) was mixed with 1 μL 100 mM MES. pH 6.5, and 50% (w/v) PEG 200 in a 24-well plate from Hampton Research (cat, no. HR1-002) at room temperature. Cubic-shaped crystals appeared in one month. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Stanford Synchrotron Radiation Lightsource (SSRL) 12-2 beamline and processed with XDS by using the autoxds script. Ho^{III}-bound LanD crystallized in the I2₁3 space group with 1 monomer in the ASU. The structure was solved using the single-wavelength anomalous diffraction (SAD) method. Using a dataset collected at 12.7 keV (0.97949 Å). HySS identified 3 Ho^{III} sites that were subsequently used to obtain phases in phenix, autosol. The initial figure of merit (FOM) and Bayesian CC was 0.681 and 0.71, respectively. An initial model was generated with phenix.autobuild with subsequent rounds of manual modification and refinement in Coot and phenix.refine. In the final stages of model refinement, anisotropic displacement parameters (ADP) and occupancies were refined for all Ho^{III} sites. The final refinement afforded an R_{work}/R_{free} of 0.221/0.223. The final model consists of residues 32-91, one Ho^{III} ion, and 8 water molecules. Of the residues modeled. 100% are in allowed or preferred regions as indicated by Ramachandran statistical analysis. Model validation was performed with the Molprobity server. Figures were prepared using the PyMOL molecular graphics software package (Schrödinger, LLC).

### Apo-LanD X-Ray Crystallography Data Collection and Structure Determination.

Purified M. extorquens LanD was exchanged into 20 mM MOPS and 20 mM KCl, pH 7, prior to crystallization. Crystals were obtained by using the sitting drop vapor diffusion method, in which 1 μL of protein solution (19.6 mg/mL) was mixed with 1 μL 0.2 M potassium fluoride, and 20% (w/v) PEG 3350 at room temperature. Thin plate-shaped crystals appeared in three days. Crystals suitable for data collection were mounted on rayon loops, soaked briefly in a cryoprotectant solution consisting of the well solution supplemented with perfluoropolyether cryo oil from Hampton Research (cat, no. HR2-814), and flash frozen in liquid N₂.

Diffraction datasets were collected at the Life Sciences Collaborative Access Team (LS-CAT) ID-G beamline and processed with the HKL2000 package. Apo-LanD crystallized in the C2 space group with two monomers. The structure was solved using the molecular replacement in PHENIX. The coordinates of La^{III}-bound LanD were used as a search model after deletion of metal ions and water molecules. The final model afforded an FOM of 0.81 and R_{work}/R_{free} of 0.205/0.243. The final model consists of residues 32-91, and 194 water molecules. Of the residues modeled, 100% are in allowed or preferred regions as indicated by Ramachandran statistical analysis. Model validation was performed with the Molprobity server. Figures were prepared using the PyMOI, molecular graphics software package (Schrödinger, LLC).

### Isothermal Titration Calorimetry.

The dissociation constants for the dimers of apo and La-, Ce-, Nd-, Eu-, and Ho-bound LanD were determined by dilutive additions of a concentrated protein stock, using isothermal titration calorimetry on a TA Instruments Low-volume Auto Affinity isothermal titration calorimeter. The syringe contained 800 μM protein (apo). 800 μM protein with 0.5 equivalents of Ln^{III} (Ln=La or Ce), or 1 mM protein with 0.5 equivalents of Ln^{III} (Ln=Nd, Eu, or Ho), and the cell contained 185 μL of a matched buffer (30 mM MOPS, 100 mM KCl, pH 7.0). Titrations were carried out at 25° C. Titrations consisted of a first 0.2-μL injection followed by 20×2-μL injections with stirring at 125 rpm and 180 s equilibration time between injections. The data were fitted using NanoAnalyze using the Dimer Dissociation model, yielding the dimer dissociation constant (K_{dimer}), enthalpy of dissociation (ΔH) and entropy of dissociation (ΔS). Similarly, dissociation constants for the dimers of La-, Ce-, Pr-, and Nd-bound E75Q/E78A were determined. The syringe contained 250 μM protein with 0.5 equivalents of La^{III} or 150 μM protein with 0.5 equivalents of Ln^{III} (Ln=Ce, Pr, or Nd).

Binding of Eu^{III} to LanD was characterized using a TA Instruments Low-volume Auto Affinity isothermal titration calorimeter. The ITC cell contained 15 μM LanD in Chelex-treated 30 mM MOPS, 100 KCl, pH 7.0. Titrations were carried out at 25° C. The titrant syringe contained 120 μM Eu^{III} prepared in the same buffer. Titrations consisted of 26×1.6 μL injections. The equilibration times were 180 s between injections, and the sample cell was stirred at 125 rpm. The heats of dilution were determined by titrating the same metal solutions into matched buffer without protein. The corrected heats were determined by subtracting the heats of dilution from the protein data. The data were fitted using NanoAnalyze using the Independent model, yielding the dissociation constant (K_{d1}), stoichiometry (n), enthalpy of dissociation (ΛH), and entropy of dissociation (ΛS). For measurements of binding of Eu^{III} to LanD-E75Q, the ITC cell contained 20 μM protein in Chelex-treated 30 mM MOPS, 100 mM KCl, pH 7.0 buffer with the titrant syringe containing 160 μM Eu^{III} in the same buffer.

For characterization of binding of LanM to LanD, the ITC cell contained 30 μM LanD in Chelex-treated 30 mM MOPS, 100 mM KCl, pH 7.0, and the titrant syringe contained 240 μM apo-Mex-LanM, Sm₃-Mex-LanM, or apo-Hans-LanM, prepared in the same buffer. Heats of dilution were determined by titrating the same titrant syringe solutions into matched buffer. Other experimental conditions and analysis were the same as for titration of Eu^{III} into LanD.

Uncertainties for ITC measurements are given as the larger of standard deviations of the means from at least three replicate experiments, or of uncertainties (95% confidence intervals) from the fitting of those replicates, following error propagation. For these experiments and other K_{d} determinations, the potential for weak binding of buffer to the metal ions has not been assessed, so the K_{dS} are apparent.

### Xylenol Orange Competition.

Samples of 10 μM protein and 5 μM xylenol orange (XO) were prepared in Chelex-treated 20 mM MES, 100 mM KCl, pH 6.0 buffer. Titrations were carried out through addition of 0.5 μL of titrant (from 1 mM Ln^{III} stock solutions: Ln=La, Nd, or Eu). Absorbance spectra were collected between 300 and 800 nm for each addition of Ln until there was no further change in the Ln-XO feature at 575 nm. Spectra were corrected for dilution. Titrations were compared to a control titration of La^{III} into a solution of 5 μM XO in buffer.

### Fluorescence Measurements, a) General Methods.

All fluorescence data were collected with a Fluorolog-QM fluorometer in configuration 75-21-C (Horiba Scientific) equipped with a double monochromator on the excitation arm and single monochromator on the emission arm. A 75-W xenon lamp was used as the light source for steady-state measurements and a pulsed xenon lamp was used for lifetime measurements. All fluorescence data were collected in quartz cuvettes and the emission was collected at 90° relative to the excitation. Lifetimes were fitted using FelixFL software (Horiba Scientific).

Fluorescence Measurements, b) Lifetime Measurements in D₂O—H₂O Mixtures.

These measurements were carried out using established methods. Solutions of 20 μM or 350 μM LanD with 0.5 equivalents Eu^{III} were prepared in 100% H₂O matrix (Buffer: 20 mM Tris, 100 mM KCl, pH 7.0). This initial 100% H₂O Eu^{III}-LanD₂ mixture was split in half (2×1.5 mL aliquots) and dehydrated by lyophilization. The residual solid was rehydrated in an equivalent amount of H₂O or 99.9% D20, lyophilized again, and resuspended a second time in an equivalent amount of H₂O or 99.9% D20. These two Eu^{III}-LanD₂ solutions (100% H₂O or ˜99.9% D₂O) were mixed in varying ratios to produce D₂O contents of 0%, 25%, 50%, 75%, and 99.9%. For each mixture, the lifetime was measured (λₑₓ=394 nm, λₑₘ=615 nm) with 2000 shots over a time span of 2500 μs.

At 20 μM LanD, where the monomer dominates and Eu^{III} is fully protein bound, q=4.1, while at 350 μM, q decreases to 2.6 (FIG. 6A). Assuming that the holodimer has no coordinated solvent (q=0) based on the X-ray structure of Eu^{III}-LanD, monomer has q=4.1, and given q=2.6 at 350 μM LanD, ˜65% of the protein units are monomeric in solution under these conditions. This value was used to determine K_{dimer} for Eu^{III}-LanD using the equation 2 [M]²+K_{dimer}([M]-[P])=0, where [M] is the monomer concentration and [P] is the total protein concentration.

For measurements in the presence of small molecules, solutions of 20 μM LanD with 0.9 equivalents Eu^{III} were prepared in the presence and absence of 200 μM of citrate, 4-hydroxy benzamide, or 3,4-dihydroxy benzamide in 100% H₂O matrix (Buffer: 30 mM MOPS, 100 mM KCl, pH 7.0). Control solutions were prepared without LanD. Eu^{III} luminescence lifetimes were determined as described above.

### Fluorescence Measurements, c) Small Molecule Titrations.

A solution of 15 μM LanD loaded with one equivalent (15 μM) of Eu^{III} was prepared in Chelex-treated 30 mM MOPS, 100 mM KCl, pH 7.0 buffer. Steady state fluorescence emission spectra were collected with settings: λₑₓ=394 nm with 7 nm slit widths, λₑₘ 560-650 nm with 15 nm slit widths, integration time=1 s, step size=0.5 nm. Titrations were carried out through addition of 0.5 μL of titrant (from concentrated stock solutions). The baseline of each spectrum was fitted to a high order polynomial using OriginLab and subtracted out. Spectra were corrected for dilution.

### Fluorescence Measurements, d) 1:1 Ln^{III} Competition Measurements.

Solutions of protein (5 μM WT or 50 μM E75Q) were loaded with 4 equiv, of Eu^{III} and 4 equiv, of another Ln^{III} (Ln=La, Ce, Pr, Nd, Sm, or Gd) and prepared in Chelex-treated 30 mM MOPS, 100 mM KCl, pH 7.0. Time-resolved fluorescence emission spectra were collected with settings: λₑₓ=394 nm with 7 nm slit widths, λₑₘ 560-650 nm with 15 nm slit widths. 150 μs delay, 1100 μs end time, 300 shots, step size=0.5 nm. Emission spectra were fitted to two Gaussian peaks using the Gauss function in OriginLab. Fractional binding of Eu^{III} and the competing Ln^{III} was determined through comparison of emission intensity at 617 nm of the competition sample to a 0% bound control (4 equiv, of Eu^{III} in buffer) and a 100% bound control (5 μM WT or 50 μM E75Q with 4 equiv, of Eu^{III} in buffer). K_{d1} values were calculated using Equation 1.

$${K}_{d 1 , Ln} = \frac{{{{K}_{d 1 , Eu} [ Ln ]}_{free} [ Eu ]}_{bound}}{{{[ Eu ]}_{free} [ Ln ]}_{bound}} \qquad ( 1 )$$

### Separation Experiments.

Ten mL samples containing protein and a mixture of Ln^{III} ions was incubated for 15 min in a Multi Tube Rotator. Amicon Ultra-15 centrifugal filters were washed with buffer prior to sample application. Separations containing wt LanD utilized 3 kDa MWCO filters and separations containing E75Q/E78A utilized 10 kDa MWCO filters. Samples were applied to filters after incubation and concentrated by centrifugation for 10 min intervals at 4000×g at 20° C. until the retentate was concentrated 20-fold (final volume ˜0.5 mL). Total volume of retentate and flowthrough were measured and Ln concentration in each was analyzed via inductively coupled plasma-mass spectrometry (ICP-MS, Laboratory for Isotopes and Metals in the Environment Facility). Distribution coefficients and separation factors were calculated utilizing equations 2 and 3 respectively.

$${D}_{Ln} = \frac{\% recovery of Ln in retentate}{\% recovery of Ln in flowthrough} \qquad ( 2 )$$

$${SF}_{Ln 1 / Ln 2} = \frac{{D}_{Ln 1}}{{D}_{Ln 2}} \qquad ( 3 )$$

### Investigation of Potential Ternary Complex Formation in LanD.

Given LanD's partial protein ligation and relatively weak Ln^{III} ion affinity, we considered whether the coordination sphere of a LanD-bound Ln^{III} ion might be completed in a ternary complex with another multidentate ligand, such as a secreted metallophore or a degradation product thereof. Because lanthanides, unlike Fe^{III}, cannot be readily reduced, a metallophore would likely need to be hydrolyzed for Ln^{III} ion mobilization after uptake, as for Fe^{III}-enterobactin. A small molecule called methylolanthanin, closely related to the known siderophore rhodopetrobactin B, has been isolated from culture medium of M. extorquens and has been suggested to be involved in lanthanide uptake. These molecules comprise a central citrate moiety linked via modified 4,4′-diaminodibutylamine arms to either 4-hydroxybenzoyl (methylolanthanin) or 3,4-dihydroxy benzoyl (rhodopetrobactin B) moieties (FIG. 44C). Thus, we assessed the potential of the putative key metal-binding functionalities of these molecules—citrate, 4-hydroxybenzamide, and 3.4-dihydroxy benzamide—to form ternary complexes with LanD. None of these three fragments at 10-fold excess significantly altered the q value of Eu^{III}-LanD, suggesting neither ternary complex formation nor outcompetition of LanD for Eu^{III} binding: only at millimolar concentrations did citrate and 3,4-dihydroxy benzamide outcompete LanD for Eu^{III} binding (FIGS. 44-46). Therefore, despite its rather low affinity (compared to LanM, for example), LanD can still bind Ln^{III} ions in the presence of these small-molecule ligands that may exist in the periplasm.

TABLE 4
Data collection and refinement statistics for the X-ray structures of apo-, La^{III}-, Ce^{III}-,
Eu^{III}-, and Ho^{III}-LanD. Statistics for the highest resolution shell are shown in parentheses.

| | Apo | | La | | Ce | | Eu | | Ho | |
|---|---|---|---|---|---|---|---|---|---|---|
| Wavelength (Å) | 0.97857 | | 0.97857 | | 0.97946 | | 0.92011 | | 0.97949 | |
| Resolution range | 38.39-1.65 | | 38.85-1.91 | | 31.88-1.801 | | 31.98-1.41 | | 39.08-1.38 | |
| | (1.71-1.65) | | (1.98-1.91) | | (1.865-1.801) | | (1.46-1.41) | | (1.42-1.38) | |
| Space group | C 2 | | I 2₁ 3 | | I 2₁ 3 | | I 2₁ 3 | | I 2₁ 3 | |
| Unit cell | 76.78 23.51 76.45 | | 77.71 77.71 77.71 | | 78.09 78.09 78.09 | | 78.33 78.33 78.33 | | 78.36 78.36 78.36 | |
| | 90 120.02 90 | | 90 90 90 | | 90 90 90 | | 90 90 90 | | 90 90 90 | |
| Total | 101419 | (7082) | 195787 | (5170) | 300321 | (30119) | 110367 | (3795) | 649095 | (45835) |
| reflections | | | | | | | | | | |
| Non-anomalous | 14086 | (1073) | 5828 | (281) | 7504 | (742) | 14522 | (575) | 16505 | (1210) |
| unique | | | | | | | | | | |
| reflections | | | | | | | | | | |
| Multiplicity | 7.2 | (6.6) | 33.6 | (18.4) | 40.0 | (40.6) | 7.6 | (6.6) | 39.3 | (37.9) |
| Completeness (%) | 96.5 | (75.1) | 94.1 | (45.8) | 100.0 | (100.0) | 93.3 | (37.9) | 100.0 | (100.0) |
| Mean I/sigma(I) | 28.4 | (2.7) | 14.7 | (2.0) | 27.1 | (12.7) | 24.7 | (1.5) | 21.8 | (1.3) |
| Wilson B-factor | 13.29 | | 16.83 | | 22.83 | | 12.33 | | 19.75 | |
| R-meas | 0.055 | (0.624) | 0.255 | (1.62) | 0.123 | (0.248) | 0.096 | (0.79) | 0.088 | (3.39) |
| CC ½ | 1.000 | (0.900) | 1.000 | (0.659) | 0.998 | (0.994) | 0.997 | (0.634) | 1.000 | (0.477) |

| Reflections used | 14082 | 5828 | 7504 | 14248 | 16502 |
|---|---|---|---|---|---|
| in refinement | | | | | |
| Reflections used | 699 | 281 | 380 | 1422 | 1640 |
| for R-free | | | | | |
| R-work | 0.2119 | 0.2150 | 0.1873 | 0.1972 | 0.2210 |
| R-free | 0.2570 | 0.2552 | 0.2464 | 0.2049 | 0.2234 |
| Number of non- | 1075 | 507 | 499 | 501 | 483 |
| hydrogen atoms | | | | | |
| macromolecules | 889 | 466 | 461 | 490 | 474 |
| ligands | 0 | 1 | 1 | 1 | 1 |
| solvent | 186 | 40 | 37 | 10 | 8 |
| Protein residues | 118 | 60 | 60 | 60 | 60 |
| RMS(bonds) | 0.005 | 0.009 | 0.008 | 0.008 | 0.008 |
| RMS(angles) | 0.82 | 0.87 | 0.79 | 0.96 | 0.92 |
| Ramachandran | 100 | 98.28 | 98.28 | 100 | 100 |
| favored (%) | | | | | |
| Ramachandran | 0.00 | 1.72 | 1.72 | 0.00 | 0.00 |
| allowed (%) | | | | | |
| Ramachandran | 0.00 | 0.00 | 0.00 | 0.00 | 0.00 |
| outliers (%) | | | | | |
| Rotamer outliers | 1.16 | 0.00 | 2.22 | 0.00 | 2.13 |
| (%) | | | | | |
| Clashscore | 1.12 | 1.06 | 1.08 | 0.99 | 3.14 |
| Average B-factor | 27.58 | 23.62 | 28.85 | 23.38 | 35.93 |
| macromolecules | 27.02 | 23.03 | 28.71 | 23.14 | 35.76 |
| ligands | — | 14.79 | 15.84 | 10.72 | 20.37 |
| solvent | 30.26 | 30.65 | 30.92 | 36.31 | 47.87 |
| PDB code | 9C8W | 9C8X | 9C8Y | 9C8Z | 9C90 |

TABLE 5
Assessment by isothermal titration calorimetry of dissociation
constants for dimerization (K_{dimer}) for the apoproteins
of wild-type LanD and its D39S and R47K variants. Conditions:
30 mM MOPS, 100 mM KCl, pH 7.0, 25° C.

| | K_{dimer}, μM | ΔH, kcal/mol | ΔG, kcal/mol | ΔS, cal/mol · K |
|---|---|---|---|---|
| WT | 609 ± 67 | 2.18 ± 0.18 | 4.39 ± 0.06 | −7.39 ± 0.43 |
| D39S | 795 ± 64 | 2.58 ± 0.07 | 4.23 ± 0.02 | −5.53 ± 0.25 |
| R47K | 1060 ± 250 | 2.52 ± 0.32 | 3.44 ± 1.04 | −5.18 ± 1.03 |

TABLE 6
Full ITC parameters for dimerization dissociation constant measurements
of wild-type LanD in the presence of 0.5 equiv. each Ln^{III} ion.
Conditions: 30 mM MOPS, 100 mM KCl, pH 7.0, 25° C.

| | K_{dimer}, μM | ΔH, kcal/mol | ΔG, kcal/mol | ΔS, cal/mol · K |
|---|---|---|---|---|
| La | 117 ± 34 | 0.12 ± 0.04 | 5.38 ± 0.20 | −17.66 ± 0.63 |
| Ce | 200 ± 10 | 0.33 ± 0.04 | 5.05 ± 0.03 | −15.81 ± 0.17 |
| Nd | 253 ± 52 | 0.14 ± 0.04 | 4.91 ± 0.13 | −16.02 ± 0.56 |
| Eu | 1420 ± 405 | 1.38 ± 0.06 | 3.90 ± 0.18 | −8.44 ± 0.51 |
| Ho | 672 ± 139 | 1.55 ± 0.26 | 4.34 ± 0.13 | −9.36 ± 0.87 |

TABLE 7
Full ITC parameters for Eu^{III}-LanD K_{d1} measurement. Conditions:
30 mM MOPS, 100 mM KCl, pH 7.0; 15 μM LanD in titration cell
and 120 μM Eu^{III} in titration syringe.

| | K_{d}, nM | n | ΔH, kcal/mol | ΔS, cal/mol · K |
|---|---|---|---|---|
| Eu | 342 ± 86 | 1.04 ± 0.03 | 0.86 ± 0.04 | 32.5 ± 0.6 |

TABLE 8
Affinity for monomer (K_{d1}) and dimerization
K_{dS} for wild-type LanD.

| | K_{d1} (μM)^{a} | K_{dimer} (μM)^{c} |
|---|---|---|
| La | 1.80 ± 0.27 | 117 ± 34 |
| Ce | 0.90 ± 0.13 | 200 ± 10 |
| Pr | 0.53 ± 0.08 | ND |
| Nd | 0.27 ± 0.04 | 253 ± 52 |
| Sm | 0.32 ± 0.05 | ND |
| Eu | 0.34 ± 0.09^{b} | 1420 ± 405 |
| Gd | 0.65 ± 0.10 | ND |

^{a}K_{d1} values were determined by competitive titration against Eu^{III}.
^{b}Eu^{III} K_{d1} was determined by ITC. See Table 7 for full analysis of K_{d1.Eu} measurements.
^{c}K_{dimer} values were determined by ITC. See Table 6 for full analysis of K_{dimer} measurements.
Conditions: 30 mM MOPS, 100 mM KCl, pH 7.0, 25° C.

TABLE 9
Full ITC parameters for Eu^{III}-LanD-E75Q K_{d1} measurement.
Conditions: 30 mM MOPS, 100 mM KCl, pH 7.0; 20 μM LanD-E75Q
in titration cell and 160 μM Eu^{III} in titration syringe.

| | K_{d1}, nM | n | ΔH, kcal/mol | ΔS, cal/mol · K |
|---|---|---|---|---|
| Eu | 879 ± 379 | 0.86 ± 0.11 | 1.71 ± 0.16 | 33.60 ± 1.46 |

TABLE 10
Full ITC parameters for dimerization dissociation constant measurements
of LanD-E75Q/E78A in the presence of 0.5 equiv. each Ln^{III} ion.
Conditions: 30 mM MOPS, 100 mM KCl, pH 7.0, 25° C.

| | K_{dimer}, μM | ΔH, kcal/mol | ΔG, kcal/mol | ΔS, cal/mol · K |
|---|---|---|---|---|
| La | 14.8 ± 8.6 | −7.31 ± 1.50 | 6.68 ± 0.45 | −46.95 ± 6.54 |
| Ce | 7.8 ± 3.1 | −5.10 ± 0.71 | 7.00 ± 0.22 | −40.57 ± 0.50 |
| Pr | 3.1 ± 1.6 | −5.92 ± 2.4 | 7.58 ± 0.35 | −45.29 ± 9.24 |
| Nd | 3.1 ± 1.1 | −6.45 ± 1.00 | 7.52 ± 0.06 | −46.85 ± 0.33 |

TABLE 11
Distribution coefficients from separation experiment consisting
of equimolar La^{III}, Ce^{III}, Pr^{III}, and Nd^{III} ions.

| | Distribution Coefficient |
|---|---|
| La | 0.46 ± 0.04 |
| Ce | 1.39 ± 0.13 |
| Pr | 2.32 ± 0.22 |
| Nd | 3.34 ± 0.30 |

TABLE 12
Full ITC parameters for titration of apo-LanM (240 μM)
into apo-LanD (30 μM). Conditions: 30 mM MOPS, 100
mM KCl, pH 7.0, 25° C.

| | K_{d}, μM | n | ΔH, kcal/mol | ΔS, cal/mol · K |
|---|---|---|---|---|
| LanD/LanM | 4.0 ± 1.9 | 1.2 0.2 | 1.69 ± 0.08 | 30.6 ± 0.9 |

TABLE 13
Comparison of SFs for La^{III}, Ce^{III}, Pr^{III}, and Nd^{III} ions for some
selected ligand systems. ND denotes not determined. In certain
systems noted below, the aqueous phase commonly contains a “hold-
back reagent” such as EDTA or lactic acid to enhance SFs.

| | SF(Ce/La) | SF(Pr/Ce) | SF(Nd/Pr) | Reference |
|---|---|---|---|---|
| LanD (pH 6)^{a} | 3.0 ± 0.4 | 1.7 ± 0.2 | 1.4 ± 0.2 | This work |
| Mex-LanM column (pH 5) | 1.8 | 1.3 | 1.1 | Park |
| Hans-LanM(R100K) | 1.8 | 1.1 | 0.8 | Cotruvo |
| column (pH 5) | | | | |
| PC88A (0.1M HCl)^{b} | 1.3 | 1.1 | 1.2 | Sato |
| DEHPA (0.1M HCl)^{c} | 2.1 | 1.1 | 1.1 | Sato |
| DEHPA (pH 3) | 4.8 | ND | ND | Wu |
| TODGA ^{d} | 5.1 (Pr/La) | ND | 2.5 | Jansone-Popova |
| DMDODGA ^{d} | 4.2 (Pr/La) | ND | 2.8 | Jansone-Popova |
| Macropa (pH 3) ^{e} | 15.8 | 9.6 | 7.5 | Wilson |
| Macrophosphi (pH 3) | 7.4 | 1.7 | 1.4 | Wilson |
| Macrophosphi (pH 4.6) | 45.5 | 7.7 | 2.3 | Wilson |
| BLPhen1 ^{f} | 1.7 | 0.12 | 0.15 | Jansone-Popova |
| Tripodal amido-arene^{g} | 1 | 2 | 3 | Love |

Conditions:
^{a}LanD: 5 μM, 0.8 μM each La, Ce, Pr, Nd, pH 6, 10K MWCO filtration, see Table 3. 15 min incubation followed by centrifugal filtration (<45 min).
^{b}PC88A: mono-2-ethylhexyl(2-ethylhexyl)phosphonic acid (also called EHEHPA)
^{c}DEHPA: di(2-ethylhexyl)phosphoric acid (also called HDEHP)
^{d} TODGA and DMDODGA: 3M HCl / 0.1M DGA in Isopar L with 30 vol % Exxal 13 at 25° C., 1 h
^{e} Macropa and macrophosphi: in DEHPA, o-xylene, lactic acid, sodium nitrate, pH 3 or 4.6. Macrophosphi equilibrates within 1 h but macropa requires longer, and the 24 h values are given here.
^{f} Note that BLPhen1 has reverse-size selectivity to most ligands: SF_{(La/Ce)} = 0.6, SF_{(Ce/Pr)} = 8.1, and SF_{(Pr/Nd)} = 6.6. 25 h, optimal performance in 0.9M HNO₃ and 1,2-dichloroethane system.
^{g}Triamidoarene: 8M HNO₃, toluene, 24 h

TABLE 14
Comparison of the amino acid sequences of LanD and variants
characterized in this study. The signal peptide is
underlined, the C-terminal YG addition is in
bold, and point mutations are bolded and in red.

| | | SEQ ID |
|---|---|---|
| | Amino acid sequence | NO: |
| WT | MMRTRTSLAV PRGFRGSALL ALVVLATPAL ADDKAACADG | 2 |
| | IAAVKARVEK LAPEAVPQKL KRALKIAERE QGEGEFDECL | |
| | EALDDAKRAL PKYG | |
| D39S | MMRTRTSLAV PRGFRGSALL ALVVLATPAL ADDKAACA [symbol] G | 3 |
| | IAAVKARVEK LAPEAVPQKL KRALKIAERE QGEGEFDECL | |
| | EALDDAKRAL PKYG | |
| R47K | MMRTRTSLAV PRGFRGSALL ALVVLATPAL ADDKAACADG | 4 |
| | IAAVKA [symbol] VEK LAPEAVPQKL KRALKIAERE QGEGEFDECL | |
| | EALDDAKRAL PKYG | |
| E75Q | MMRTRTSLAV PRGFRGSALL ALVVLATPAL ADDKAACADG | |
| | IAAVKARVEK LAPEAVPQKL KRALKIAERE QGEG [symbol] FDECL | 5 |
| | EALDDAKRAL PKYG | |
| E75Q/ | MMRTRTSLAV PRGFRGSALL ALVVLATPAL ADDKAACADG | |
| E78A | IAAVKARVEK LAPEAVPQKL KRALKIAERE QGEG [symbol] FD [symbol] CL | 10 |
| | EALDDAKRAL PKYG | |

### EXAMPLE 3

This example provides a description of proteins and peptides of the present disclosure.

### Further Characterization of Wt LanD.

Characterization of K_{d1} values of wt LanD, from 1:1 competition between Eu^{III} and other Ln^{III} ions (monitored using time resolved fluorescence) (FIG. 53). This extends the data in FIG. 6B to the end of the lanthanide series and shows that between Tm and Lu LanD again displays relative large differences in binding affinity between adjacent lanthanides. We speculate that this may reflect a contraction in coordination number somewhere to the right to Er. Conditions: 30 mM MOPS, 100 mM KCl, pH 7.0:5 μM protein: 20 μM each Ln^{III} ion.

### Further Characterization of LanD-E75Q/E78A.

FIG. 54 shows separation factors for additional binary separations using this protein, demonstrating flattening of selectivity after Sm, in line with FIG. 53.

### Characterization of LanD-E78A.

Characterization of LanD-E78A shows slight differences from LanD-E75Q/E78A that may be beneficial for separations. Xylenol orange titration shows that E78A has similar behavior as E75Q/E78A where there is only out competition of XO until ˜0.2 eq Ln (vs. ˜0.3-0.4 for E75Q/E78A) (FIG. 55A). The LanD-E78A dimer is somewhat weaker than LanD-E75Q/E78A, likely on the order of 10 μM (FIG. 55B,C).

Luminescence competition experiments (under conditions, 50 μM protein, in which LanD-E78A is expected to be nearly fully dimerized) show slightly larger affinity differences between lanthanides than with LanD-E75Q/E78A (FIG. 55D): a 5-fold higher concentration of La^{III} (˜125 μM) was needed to displace 50% of the bound Eu^{III}, and a slightly lower (20 μM, 0.8 times) concentration of Nd^{III} was needed to displace 50% of the bound Eu^{III} (5/0.8=˜6-fold range overall). This compares with a ˜3- to 4-fold range overall for LanD-E75Q/E78A (FIG. 7B).

Therefore, these data suggested that a separation of LREs using LanD-E78A might be better than for the double variant (E75Q/E78A). The same separation conditions as used for the double variant (5 μM LanD-E78A, 1.7 μM each Ln^{III}, 10 mL, pH 6.0; concentrated 20-fold using a 10 kDa filter) were tested (FIG. 56). Within uncertainty, the performance of E78A was similar to that of E75Q/E78A. However, we note that the LanD-E78A K_{dimer} for Nd is expected to be higher than the protein concentration, whereas with the previous E75Q/E78A separation the initial protein concentration was ˜2 times that of the K_{dimer} for Nd-E75Q/E78A. Therefore, we expect that the separation factor should be improved by changing the initial protein concentration.

### Efforts to Enable LanD Monomer-Based Separations.

The wt protein exhibits promising K_{d1} differences among La-Nd but poor separation performance, which we attribute potentially to non-specific metal-binding sites (FIG. 43). We sought to ablate these sites (D77, E78, and E81, see FIG. 14), but our work above showed that the E78A mutation leads to much higher propensity for dimerization. Therefore, we designed the LanD-D77N/E81A variant. Initial XO assays did not yield clear stoichiometric information, suggesting that metal binding affinity might be comparable to that of XO (FIG. 57A). A luminescence titration yielded close to stoichiometric (˜0.8 equiv.) binding to Eu^{III} suggested tight binding of the metal to the monomer (FIG. 57B).

We proceeded to conduct luminescence competition experiments to estimate relative affinities for Ln^{III} ions (vs. Eu^{III}) (FIG. 58A). After adding 1 equiv. Eu^{III}; La^{III}, Pr^{III}, or Sm^{III} were titrated into solution and decrease in Eu^{III}-based emission was monitored. The results suggested that Pr^{III}, Sm^{III}, and Eu^{III} have fairly equivalent affinity for the monomer, while La^{III} is ˜3 times weaker than Eu^{III} (lower selectivity than wt). However, when separation studies were carried out, replicating separation conditions for wt LanD (1 μM D77N/E81A, 1 μM each Ln^{III}, 10 mL, pH 6.0; concentrated 20-fold using a 3 kDa filter), the separation factor for Pr/La was 1.8 (FIG. 58B), slightly better than for wt LanD (1.4). The binding stoichiometry of 0.8 in the retentate suggests that non-specific binding may have been disfavored but also that some dimer may still be forming. This suggests (along with work on the E78A-containing variants) that substitutions of residues near the metal-binding site in LanD with Ala may not be ideal because they facilitate dimerization by reducing steric clashes and charge repulsion.

However, these results do suggest that further pursuit of the general strategy of reducing non-specific binding while still preventing dimerization could be promising. Additional sequences have been designed for testing toward this end (see table of SEQ IDs).

Although the present disclosure has been described with respect to one or more particular embodiments and/or examples, it will be understood that other embodiments and/or examples of the present disclosure may be made without departing from the scope of the present disclosure.

1. A metal-binding protein comprising the following sequence:
(SEQ ID NO: 1)
DDKAACAX¹GIAAVKAX²VEKLAPEAVPQKLK
RALKIAEREQGEGX³FX⁴X⁵CLX⁶ALX⁷DAKRA
LPKX⁸X⁹,
wherein
X¹ is D or S;
X² is R or K;
X³ is E, Q, or M;
X⁴ is D, N, Q, or K;
X⁵ is E, N, Q, D, A, or T;
X⁶ is E, A, or Q;
X⁷ is D or E;
X⁸ is Y, W, or absent; and
X⁹ is G or absent, and
or a protein having at least 75% identity to SEQ ID NO:1, wherein the metal-binding protein is optionally disposed or affixed to a substrate, wherein when the metal-binding protein is SEQ ID NO:50 the metal-binding protein is disposed or affixed to the substrate.

2. The metal-binding protein according to claim 1, wherein the protein is disposed or affixed to the substrate.

3. The metal-binding protein according to claim 2, wherein the substrate is a bead, a membrane, a hydrogel, a protein-based material, or a porous framework.

4. The metal-binding protein according to claim 1, wherein the protein further comprises a signal sequence.

5. The metal-binding protein according to claim 4, wherein the signal sequence is MMRTRTSLAVPRGFRGSALLALVVLATPALA (SEQ ID NO:52).

6. The metal-binding protein according to claim 1, wherein the metal-binding protein is or comprises any one of the following sequences:

| | SEQ |
|---|---|
| Sequence | ID NO |
| DDKAACAXGI AAVKAXVEKL APEAVPQKLK RALKIAEREQ | 1 |
| GEGXFXXCLX ALXDAKRALP KXX | |
| MMRTRTSLAV PRGFRGSALL ALVVLATPAL ADDKAACADG | 2 |
| IAAVKARVEK LAPEAVPQKL KRALKIAERE QGEGEFDECL | |
| EALDDAKRAL PKYG | |
| MMRTRTSLAV PRGFRGSALL ALVVLATPAL ADDKAACASG | 3 |
| IAAVKARVEK LAPEAVPQKL KRALKIAERE QGEGEFDECL | |
| EALDDAKRAL PKYG | |
| MMRTRTSLAV PRGERGSALL ALVVLATPAL ADDKAACADG | 4 |
| IAAVKAKVEK LAPEAVPQKL KRALKIAERE QGEGEFDECL | |
| EALDDAKRAL PKYG | |
| MMRTRTSLAV PRGFRGSALL ALVVLATPAL ADDKAACADG | 5 |
| IAAVKARVEK LAPEAVPQKL KRALKIAERE QGEGQFDECL | |
| EALDDAKRAL PKYG | |
| MMRTRTSLAV PRGFRGSALL ALVVLATPAL ADDKAACADG | 6 |
| IAAVKARVEK LAPEAVPQKL KRALKIAERE QGEGEFDACL | |
| EALDDAKRAL PKYG | |
| MMRTRTSLAV PRGERGSALL ALVVLATPAL ADDKAACADG | 7 |
| IAAVKARVEK LAPEAVPQKL KRALKIAERE QGEGEFDNCL | |
| EALDDAKRAL PKYG | |
| MMRTRTSLAV PRGFRGSALL ALVVLATPAL ADDKAACADG | 8 |
| IAAVKARVEK LAPEAVPQKL KRALKIAERE QGEGEFDQCL | |
| EALDDAKRAL PKYG | |
| MMRTRTSLAV PRGFRGSALL ALVVLATPAL ADDKAACADG | 9 |
| IAAVKARVEK LAPEAVPQKL KRALKIAERE QGEGEFDDCL | |
| EALDDAKRAL PKYG | |
| MMRTRTSLAV PRGFRGSALL ALVVLATPAL ADDKAACADG | 10 |
| IAAVKARVEK LAPEAVPQKL KRALKIAERE QGEGQFDACL | |
| EALDDAKRAL PKYG | |
| MMRTRTSLAV PRGFRGSALL ALVVLATPAL ADDKAACADG | 11 |
| LAAVKARVEK LAPEAVPQKL KRALKIAERE QGEGQFDNCL | |
| EALDDAKRAL PKYG | |
| MMRTRTSLAV PRGFRGSALL ALVVLATPAL ADDKAACADG | 12 |
| IAAVKARVEK LAPEAVPQKL KRALKIAERE QGEGEFNECL | |
| AALDDAKRAL PKYG | |
| MMRTRTSLAV PRGFRGSALL ALVVLATPAL ADDKAACADG | 13 |
| IAAVKARVEK LAPEAVPQKL KRALKIAERE QGEGEFQECL | |
| QALDDAKRAL PKYG | |
| MMRTRTSLAV PRGFRGSALL ALVVLATPAL ADDKAACADG | 14 |
| IAAVKARVEK LAPEAVPQKL KRALKIAERE QGEGEFKECL | |
| QALDDAKRAL PKYG | |
| MMRTRTSLAV PRGFRGSALL ALVVLATPAL ADDKAACADG | 15 |
| IAAVKARVEK LAPEAVPQKL KRALKIAERE QGEGQFDTCL | |
| EALDDAKRAL PKYG | |
| MMRTRTSLAV PRGFRGSALL ALVVLATPAL ADDKAACADG | 16 |
| IAAVKARVEK LAPEAVPQKL KRALKIAERE QGEGQFDACL | |
| EALEDAKRAL PKYG | |
| MMRTRTSLAV PRGFRGSALL ALVVLATPAL ADDKAACADG | 17 |
| IAAVKARVEK LAPEAVPQKL KRALKIAERE QGEGMFDACL | |
| EALDDAKRAL PKYG | |
| DDKAACADGI AAVKARVEKL APEAVPQKLK RALKIAEREQ | 34 |
| GEGEFDECLE ALDDAKRALP KYG | |
| DDKAACASGI AAVKARVEKL APEAVPQKLK RALKIAEREQ | 35 |
| GEGEFDECLE ALDDAKRALP KYG | |
| DDKAACADGI AAVKAKVEKL APEAVPQKLK RALKIAEREQ | 36 |
| GEGEFDECLE ALDDAKRALP KYG | |
| DDKAACADGI AAVKARVEKL APEAVPQKLK RALKIAEREQ | 37 |
| GEGQFDECLE ALDDAKRALP KYG | |
| DDKAACADGI AAVKARVEKL APEAVPQKLK RALKIAEREQ | 38 |
| GEGEFDACLE ALDDAKRALP KYG | |
| DDKAACADGI AAVKARVEKL APEAVPQKLK RALKIAEREQ | 39 |
| GEGEFDNCLE ALDDAKRALP KYG | |
| DDKAACADGI AAVKARVEKL APEAVPQKLK RALKIAEREQ | 40 |
| GEGEFDQCLE ALDDAKRALP KYG | |
| DDKAACADGI AAVKARVEKL APEAVPQKLK RALKIAEREQ | 41 |
| GEGEFDDCLE ALDDAKRALP KYG | |
| DDKAACADGI ALDDAKRALP APEAVPQKLK RALKIAEREQ | 42 |
| GEGQFDACLE AAVKARVEKL KYG | |
| DDKAACADGI AAVKARVEKL APEAVPQKLK RALKIAEREQ | 43 |
| GEGQFDNCLE ALDDAKRALP KYG | |
| DDKAACADGI AAVKARVEKL APEAVPQKLK RALKIAEREQ | 44 |
| GEGEFNECLA ALDDAKRALP KYG | |
| GEGEFQECLQ ALDDAKRALP APEAVPQKLK RALKIAEREQ | 45 |
| DDKAACADGI AAVKARVEKL KYG | |
| DDKAACADGI AAVKARVEKL APEAVPQKLK RALKIAEREQ | 46 |
| GEGEFKECLQ ALDDAKRALP KYG | |
| GEGQFDTCLE ALDDAKRALP APEAVPQKLK RALKIAEREQ | 47 |
| DDKAACADGI AAVKARVEKL KYG | |
| DDKAACADGI AAVKARVEKL APEAVPQKLK RALKIAEREQ | 48 |
| GEGQFDACLE ALEDAKRALP KYG | |
| DDKAACADGI AAVKARVEKL APEAVPQKLK RALKIAEREQ | 49 |
| GEGMFDACLE ALDDAKRALP KYG | |
| DDKAACADGI AAVKARVEKL APEAVPQKLK RALKIAEREQ | 50 |
| GEGEFDECLE ALDDAKRALP K | |
| MMRTRTSLAV PRGFRGSALL ALVVLATPAL ADDKAACAXG | 52 |
| IAAVKAXVEK LAPEAVPQKL KRALKIAERE QGEGXFXXCL | |
| XALXDAKRAL PKXX | |

7. (canceled)

8. A device comprising a metal-binding protein according to claim 1.

9. The device according to claim 8, wherein the device is a filter, membrane, sensor, handheld detector, plate reader, fluorimeter, biosensor, or in-line monitor.

10. A kit comprising the metal-binding protein according to claim 1 or materials to prepare a device comprising the metal-binding protein according to claim 1.

11. A method for binding lanthanide ions and/or actinide ions to a protein comprising contacting a metal-binding protein of claim 1 with a sample comprising the lanthanide ions and/or actinide ions, wherein the lanthanide ions or actinide ions bind to one or more metal-binding proteins of claim 1.

12. The method according to claim 11, wherein the lanthanide ions and/or actinide ions are chosen from La, Ce, Pr, Nd, Pm, Sm, Eu, Gd, Tb, Dy, Ho, Er, Tm, Yb, Lu, Sc, Y, and any combination thereof.

13. The method according to claim 11, wherein the sample is drinking water, wastewater, ground water, ash ponds, aqueous extract from contaminated soil, drainage, leachate, aqueous extract or leachate from a solid waste, or a solid sample.

14. The method according to claim 11, further comprising isolating the one or more metal-binding proteins having one or more lanthanide ions and/or actinide ions bound thereto.

15. The method according to claim 10, wherein a plurality of different lanthanide ions and/or actinide ions are bound to the one or more metal-binding proteins.

16. The method according to claim 15, wherein each different lanthanide ions and/or actinide ions are separated individually from the metal-binding protein.

17. A metal-binding protein or peptide comprising one or more metal-binding motifs, wherein at least one of the one or more metal-binding motifs comprises the sequence:
(SEQ ID NO: 53)
REX¹X²EX³EX⁴DEC,
wherein,
X¹ is any amino acid;
X² is any amino acid;
X³ is any amino acid; and
X⁴ is F or Y,
or a protein having at least 75% identity thereto and
the cysteine residue forms a disulfide bond with a second cysteine elsewhere in the metal-binding protein or peptide.

18. The metal-binding protein or peptide according to claim 17, wherein X² is G, A, K, or R.

19. The metal-binding protein or peptide according to claim 17, wherein X³ is G, A, or K.

20. The metal-binding protein or peptide according to claim 17, wherein the metal-binding protein is or comprises any one of the following sequences:

| | SEQ ID |
|---|---|
| Sequence | NO |
| MMRTRTSLAV PRGERGSALL ALVVLATPAL ADDKAACADG IAAVKARVEK | 2 |
| LAPEAVPQKL KRALKIAERE QGEGEFDECL EALDDAKRAL PKYG | |
| MMRTRTSLAV PRGERGSALL ALVVLATPAL ADDKAACASG IAAVKARVEK | 3 |
| LAPEAVPQKL KRALKIAERE QGEGEFDECL EALDDAKRAL PKYG | |
| MMRTRTSLAV PRGERGSALL ALVVLATPAL ADDKAACADG IAAVKAKVEK | 4 |
| LAPEAVPQKL KRALKIAERE QGEGEFDECL EALDDAKRAL PKYG | |
| MMRTRTSLAV PRGERGSALL ALVVLATPAL ADDKAACADG IAAVKARVEK | 5 |
| LAPEAVPQKL KRALKIAERE QGEGQFDECL EALDDAKRAL PKYG | |
| DDKAACADGI AAVKARVEKL APEAVPQKLK RALKIAEREQ GEGEFDECLE | 34 |
| ALDDAKRALP KYG | |
| DDKAACASGI AAVKARVEKL APEAVPQKLK RALKIAEREQ GEGEFDECLE | 35 |
| ALDDAKRALP KYG | |
| DDKAACADGI AAVKAKVEKL APEAVPQKLK RALKIAEREQ GEGEFDECLE | 36 |
| ALDDAKRALP KYG | |
| DDKAACADGI AAVKARVEKL APEAVPQKLK RALKIAEREQ GEGQFDECLE | 37 |
| ALDDAKRALP KYG | |
| DDKAACADGI AAVKARVEKL APEAVPQKLK RALKIAEREQ GEGEFDECLE | 50 |
| ALDDAKRALP K | |

21. The metal-binding protein or peptide according to claim 17, wherein the metal-binding protein or peptide is disposed or affixed to the substrate.

22. The metal-binding protein or peptide according to claim 21, wherein the substrate is a bead, a membrane, a hydrogel, a protein-based material, or a porous framework.

23. A device comprising a metal-binding protein or peptide according to claim 17.

24. The device according to claim 23, wherein the device is a filter, membrane, sensor, handheld detector, plate reader, fluorimeter, biosensor, or in-line monitor.

25. A kit comprising the metal-binding protein or peptide according to claim 17 or materials to prepare a device comprising the metal-binding protein or peptide according to claim 17.

26. A method for binding lanthanide ions and/or actinide ions to a protein or peptide comprising contacting a metal-binding protein or peptide of claim 17 with a sample comprising the lanthanide ions and/or actinide ions binds to one or more metal-binding proteins or peptides of claim 17.

27. The method according to claim 26, wherein the lanthanide ions and/or actinide ions are chosen from La, Ce, Pr, Nd, Pm, Sm, Eu, Gd, Tb, Dy, Ho, Er, Tm, Yb, Lu, Sc, Y, and any combination thereof.

28. The method according to claim 26, wherein the sample is drinking water, wastewater, ground water, ash ponds, aqueous extract from contaminated soil, drainage, leachate, aqueous extract or leachate from a solid waste, or a solid sample.

29. The method according to claim 26, further comprising isolating the one or more metal-binding proteins having one or more lanthanide ions and/or actinide ions bound thereto.

30. The method according to claim 26, wherein a plurality of different lanthanide ions and/or actinide ions are bound to the protein or peptide.

31. The method according to claim 30, wherein each different lanthanide ions and/or actinide ions are separated individually from the metal-binding protein or peptide.
